(19) United States
(12) Patent Application Publication
(10) Pub. No.: US 2025/0263416 A1
(43) Pub. Date: Aug. 21, 2025

(54) SPPL2a INHIBITORS

(71) Applicant: Incyte Corporation, Wilmington, DE (US)

(72) Inventors: Jason Boer, Landenberg, PA (US); Joshua Hummel, Hockessin, DE (US); Yang Li, Claymont, DE (US); Unni K. Samavedam, Middletown, DE (US); Susan Harless Smith, Pennsburg, PA (US); Xiaozhao Wang, Mt. Laurel, NJ (US)

(21) Appl. No.: 19/053,606

(22) Filed: Feb. 14, 2025

Related U.S. Application Data
(60) Provisional application No. 63/553,929, filed on Feb. 15, 2024.
(60) Provisional application No. 63/555,660, filed on Feb. 20, 2024.
(60) Provisional application No. 63/632,768, filed on Apr. 11, 2024.

(51) Int. Cl. C07D 487/04; A61K 31/551; A61P 17/06; A61P 19/02; A61P 37/06

(52) U.S. Cl. CPC C07D 487/04; A61K 31/551; A61P 17/06; A61P 19/02; A61P 37/06

(57) ABSTRACT

The present disclosure relates to tricyclic compounds comprising a diazepinone moiety which are effective in inhibiting SPPL2a (signal peptide peptidase like protease 2a), to pharmaceutical compositions containing such inhibitors, and to methods of using such inhibitors and compositions.

### RELATED APPLICATIONS

This application is related to U.S. Provisional Application No. 63/553,929 filed Feb. 15, 2024, U.S. Provisional Application No. 63/555,660 filed Feb. 20, 2024, and U.S. Provisional Application No. 63/632,768 filed Apr. 11, 2024, the contents of which are incorporated herein in their entirety.

### FIELD OF THE INVENTION

The present disclosure relates to tricyclic compounds comprising a diazepinone moiety that are effective in inhibiting signal peptide peptidase such as protease 2a (SPPL2a), to pharmaceutical compositions containing such compounds, to methods for synthesizing these compounds, and to methods of using such compounds and pharmaceutical compositions.

### BACKGROUND

Signal peptide peptidase like 2A (SPPL2a) protein, an intramembrane aspartyl protease, appears to play a role in innate and adaptive immunity by cleaving specific transmembrane anchored proteins and thereby affecting the function of a variety of immune cells.

SPPL2a was initially described as the protease that cleaves the membrane-spanning portion of TNF-α in vitro, and thereby controls the release of IL-12 from dendritic cells (E. Friedmann et al., SPPL2a and SPPL2b promote intramembrane proteolysis of TNFα in activated dendritic cells to trigger IL-12 production, Nat. Cell Biol. Vol 8 (2006), pages 843-848). SPPL2a has also been shown to play a role in the development and function of antigen presenting cells such as B cells and dendritic cells. This is done through proteolytic processing of CD74, also known as the invariant chain of the major histocompatibility class II complex (MCHII)(D. Beisner et al. “The intramembrane protease SPPL2a is required for B cell and dendritic cell (DC) development and survival via cleavage of the invariant chain” J. Exp. Med. 210, pp 23-39, 2013). Antigen presentation via MCHII molecules allows differentiation of foreign antigens from self-antigens. When the immune system loses its capacity to discriminate “self” from “non-self,” autoimmune diseases may evolve.

Acting as a chaperone for MHCII complexes, CD74 prevents premature peptides from binding to the complex. In order for antigen-derived peptides to bind MHCII, CD74 is degraded by several proteases. Processing of full-length CD74 eads to the generation of the N-terminal 8 kDa CD74 (CD74-p8) transmembrane fragment, which is further processed by SPPL2a.

Inhibiting SPPL2a leads to the accumulation of the N-terminal CD74 fragment (CD74-p8) in intracellular compartments thereby inducing the death of CD74-expressing B cells and myeloid dendritic cells, which may impair the humoral immune response (J. Schneppenheim et al., The intramembrane protease SPPL2a promotes B cell development and controls endosomal traffic by cleavage of the invariant chain, J. Exp. Med., Vol. 210 (2013) pages 41-58). The accumulation of the unprocessed CD74 appears to impair T cell dependent antibody response in mice (D. Beisner et al. 2013).

Inhibition of SPPL2a protease, and the corresponding reduction of the immune system's antigen presenting capacity, may be relevant for the repression of detrimental, uncontrolled immune responses, e.g., pathological conditions where presentation of autoantigens drives pathology.

SPPL2a inhibition may also influence the proliferation of B-cell lymphomas, which appears to be associated with the expression of high levels of CD74 (Zhao et al., J Pathol Clin Res. 2019, 5 (1): 12-24). Furthermore, a chemical library screening for SPPL2a inhibitors identified that selective SPPL2a inhibition has immunomodulatory effects (Zhang, X, et al. Identification of SPPL2a Inhibitors by Multiparametric Analysis of a High-Content Ultra-High-Throughput Screen. SLAS Discov. (2017) Oct; 22 (9): pages 1106-1119).

Accordingly, there is a need for new potent and generally selective inhibitors of SPPL2a to treat diseases and/or conditions, especially of the immune system.

### SUMMARY

In one aspect provided herein is a compound of the Formula (I), or a pharmaceutically acceptable salt thereof,

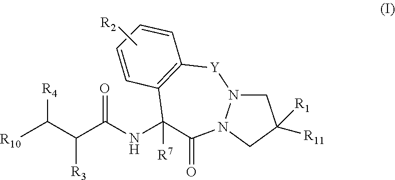

wherein:
Y is CH₂ or C═O;
R₁ is H, C₁-C₆alkyl or halo;
R₂ is H, halo, C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₄ is H, C₁-C₆alkyl or C₁-C₆alkyl-phenyl;
R₁₀ is —NHC(═O)R₅, —C(═O)NHR, or a 9 or 10 membered bicyclic heteroaryl having 1 to 4 heteroatoms as ring members each independently selected from N, O and S, wherein the bicyclic heteroaryl is unsubstituted or the bicyclic heteroaryl is substituted with one or more R₆;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by one or more D or C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC₁-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo;
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy; and
xxi) —C(═O)NH₂;
each R₆ is independently selected from C₁-C₆alkyl, C₁-C₆alkoxy, C₁-C₆haloalkyl, cyano and halo; and
R₁₁ is H, C₁-C₆alkyl or halo; or
R₁ and R₁₁ together with the carbon atom to which they are attached, may form a 3 to 6 membered carbocyclic ring;
R⁷ is selected from D, CD₃, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, (4-10 membered heterocycloalkyl)-C₁₋₆ alkylene, CN, C(O)R^{b7}, C(O)NR^{c7}R^{d7}, C(O)NR^{c7}(ORd^{d7}), C(O)OR^{d7}, C(═NR^{e7})R^{b7}, C(═NR^{e7})NR^{c7}R^{d7}, S(O)R^{b7}, S(O)NR^{c7}R^{d7}, S(O)₂R^{b7}, and S(O)₂NR^{c7}R^{d7}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R⁷ are each optionally substituted with 1, 2, 3, 4, 5, 6, 7, or 8 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R^{a7}, R^{c7}, and R^{d7} are each optionally substituted with 1, 2, 3, 4, 5, 6, 7, or 8 independently selected R^{7A} substituents;
or any R^{c7} and R^{d7} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{7A} substituents;
each R^{b7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R^{b7} are each optionally substituted with 1, 2, 3, 4, 5, 6, 7, or 8 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene;
R^{7A} is selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, (4-7 membered heterocycloalkyl)-C₁₋₆ alkylene, CN, NO₂, OR^{a71}, SR^{a71}, NHOR^{a71}, C(O)R^{b71}, C(O)NR^{c71}R^{d71}, C(O)NR^{c71} (OR^{a71}), C(O)OR^{d71}, OC(O)R^{b71}, OC(O)NR^{c71}R^{d71}, NR^{e71}R^{d71}, NR^{c71}NR^{c71}R^{d71} NR^{c71}C(O)R^{b71}, NR^{e71}C(O)OR^{d71}, NR^{e71}C(O)NR^{e71}R^{d71}, C(═NR^{e71})R^{b71}, C(═NR^{e71})NR^{e71}R^{d71}, NR^{c71}C(═NR^{e71})NR^{e71}R^{d71}, NR^{e71}C(═NR^{e71})R^{b71}, NR^{e71}S(O)R^{b71}, NR^{e71}S(O)NR^{e71}R^{d71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, and OS(O)₂R^{b71}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene of R^{7A} are each optionally substituted with 1, 2, 3, or 4 independently selected R^{M} substituents;
each R^{7A} is selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, (4-7 membered heterocycloalkyl)-C₁₋₆ alkylene, CN, NO₂, OR^{a71}, SR^{a71}, NHOR^{a71}, C(O)R^{b71}, C(O)NR^{c71}R^{d71}, C(O)NR^{c71} (OR^{a71}), C(O)OR^{d71}, OC(O)R^{b71}, OC(O)NR^{c71}R^{d71}, NR^{e71}R^{d71}, NR^{c71}NR^{c71}R^{d71} NR^{a71}C(O)R^{b71}, NR^{c71}C(O)OR^{d71}, NR^{c71}C(O)NR^{c71}R^{d71}, C(═NR^{e71})R^{b71}, C(═NR^{e71})NR^{e71}R^{d71}, NR^{c71}C(═NR^{e71})NR^{c71}R^{d71}, NR^{e71}C(═NR^{e71})R^{b71}, NR^{c71}S(O)R^{b71}, NR^{e71}S(O)NR^{e71}R^{d71}, NR^{c71}S(O)₂R^{b71}, NR^{c71}S(O)(═NR^{e71})R^{b71}, NR^{c71}S(O)₂NR^{e71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, and OS(O)₂R^{b71}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{7A} are each optionally substituted with 1, 2, 3, or 4 independently selected R^{M} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{a71}, R^{c71}, and R^{d71} are each optionally substituted with 1, 2, 3, or 4 independently selected R^{M} substituents;
or any R^{c71} and R^{d71} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b71} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{b71} are each optionally substituted with 1, 2, 3, or 4 independently selected R^{M} substituents;
each R^{e71} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene;
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene.

In another aspect, provided herein is a compound of Formula (Ia) or a pharmaceutically acceptable salt or stereoisomer thereof,

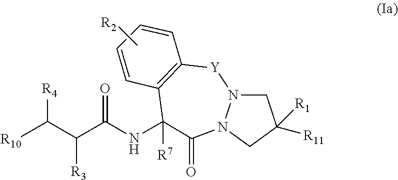

wherein:
Y is CH₂ or C═O;
R₁ is H, C₁-C₆alkyl or halo;
R₂ is C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₄ is H, C₁-C₆alkyl or C₁-C₆alkyl-phenyl;
R₁₀ is —NHC(═O)R₅, —C(═O)NHR, or a 9 or 10 membered bicyclic heteroaryl having 1 to 4 heteroatoms as ring members each independently selected from N, O and S, wherein the bicyclic heteroaryl is unsubstituted or the bicyclic heteroaryl is substituted with one or more R₆;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by one or more D or C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo;
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy; and
xxi) —C(═O)NH₂;
each R₆ is independently selected from C₁-C₆alkyl, C₁-C₆alkoxy, C₁-C₆haloalkyl, cyano and halo; and
R₁₁ is H, C₁-C₆alkyl or halo; or
R₁ and R₁₁ together with the carbon atom to which they are attached, may form a 3 to 6 membered carbocyclic ring;
R⁷ is H.

In yet another aspect, provided herein is a method of treating a condition selected from hidradenitis suppurativa, arthritis, cutaneous lupus, a pemphigus disorder, ANCA vasculitis, diabetes, dermatomyositis, primary biliary cirrhosis, polymyositis, temporal arteritis, alopecia areata, autoimmune hemolytic anemia, insulin resistance, a metabolic disease, fatty liver disease, hyperlipidemia, atherosclerosis, hypertension, stroke, gall bladder disease, and obesity in a subject in need thereof, comprising administering to the subject a therapeutically effective amount of N²,4-dimethyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl)amino)propyl)thiazole-2,5-dicarboxamide, or a pharmaceutically acceptable salt thereof.

In another aspect, provided herein is a pharmaceutical composition comprising a therapeutically effective amount of a compound provided herein, or a pharmaceutically acceptable salt thereof, and one or more pharmaceutically acceptable carriers.

In another aspect, provided herein is a method of treating a disease or disorder mediated by the activity of signal peptide peptidase like protease 2a (SPPL2a), wherein the method comprises administering to a subject in need of such treatment a compound of the present disclosure, or a pharmaceutically acceptable salt.

In another aspect, provided herein is a method of treating an autoimmune disease in a subject in need thereof, wherein the method comprises administering to the subject a therapeutically effective amount of a compound of the present disclosure, or a pharmaceutically acceptable salt thereof.

In another aspect, provided herein is a use of a compound of the present disclosure, or a pharmaceutically acceptable salt thereof, in the manufacture of a medicament for the treatment of a disease or disorder associated with or mediated by the activity of signal peptide peptidase like protease 2a (SPPL2a).

In another aspect, provided herein is a use of a compound of the present disclosure, or a pharmaceutically acceptable salt thereof, in the manufacture of a medicament for the treatment of an autoimmune disease.

In another aspect, provided herein is the use of a compound of the present disclosure, or a pharmaceutically acceptable salt thereof, in the treatment of a disease or disorder associated with or mediated by the activity of signal peptide peptidase like protease 2a (SPPL2a).

In another aspect, provided herein is the use of a compound of the present disclosure, or a pharmaceutically acceptable salt thereof, in the treatment of an autoimmune disease.

In another aspect, provided herein is a compound of the present disclosure, or a pharmaceutically acceptable salt thereof, for use in the treatment of a disease or disorder associated with of mediated by the activity of signal peptide peptidase like protease 2a (SPPL2a).

In another aspect, provided herein is a compound of the present disclosure, or a pharmaceutically acceptable salt thereof, for use in the treatment of lymphomas.

### BRIEF DESCRIPTION OF THE DR^{A}WINGS

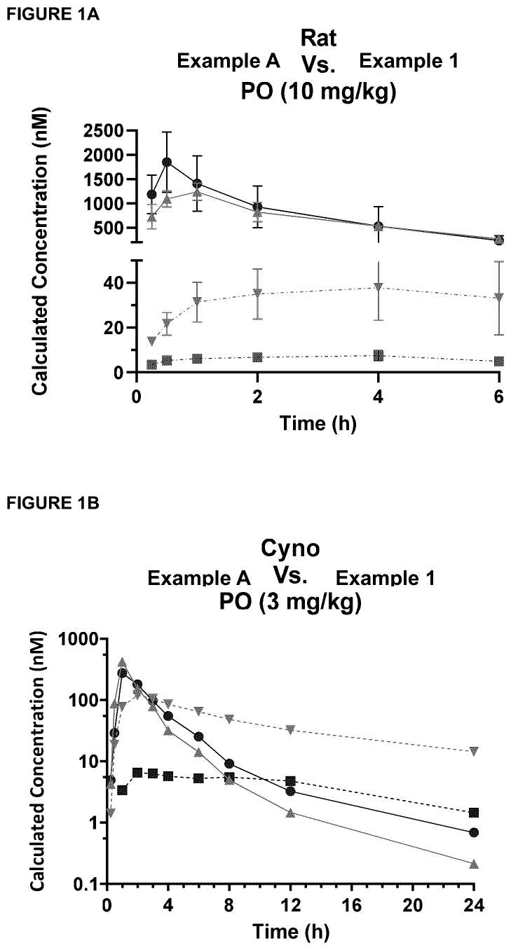

FIGS. 1A, 1B, and 1C show a reduction of in vivo epimerization for Example 1 compared to Example A in rat, monkey, and dog models, respectively.

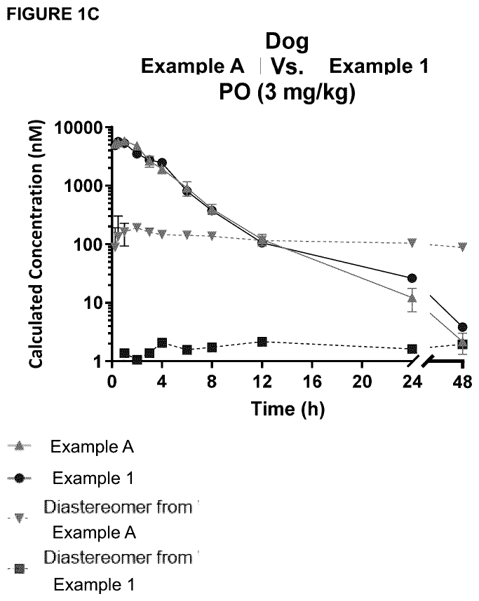

FIG. 2 shows that nuclear translocation of TNFα-eGFP is inhibited by the selective SPPL2a inhibitors Example 1 and Example A.

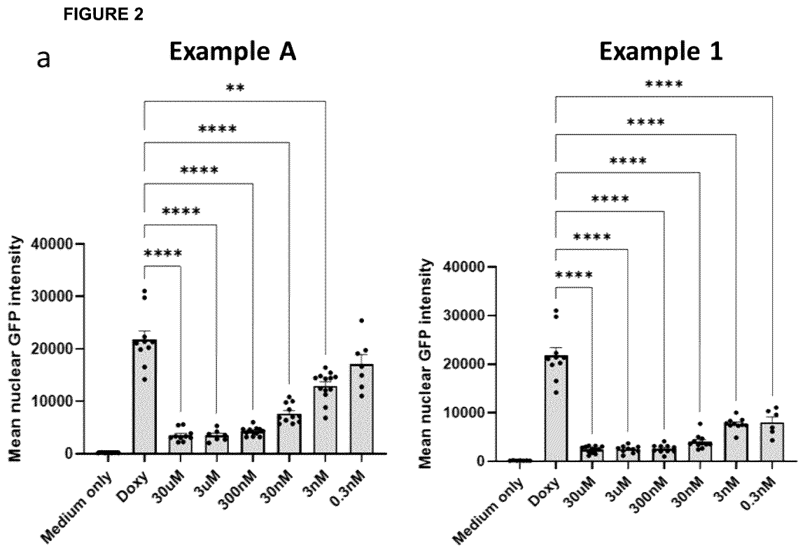

FIG. 3 shows SPPL2a activity inhibition in the SPPL2a-U2OS reporter cells by Example 1 and Example A.

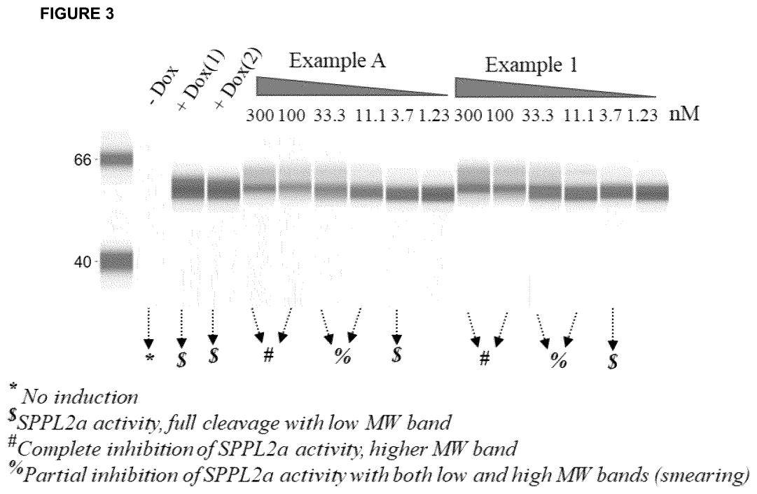

FIG. 4 shows the effect of Example 1 and Example A on dendritic cell maturation.

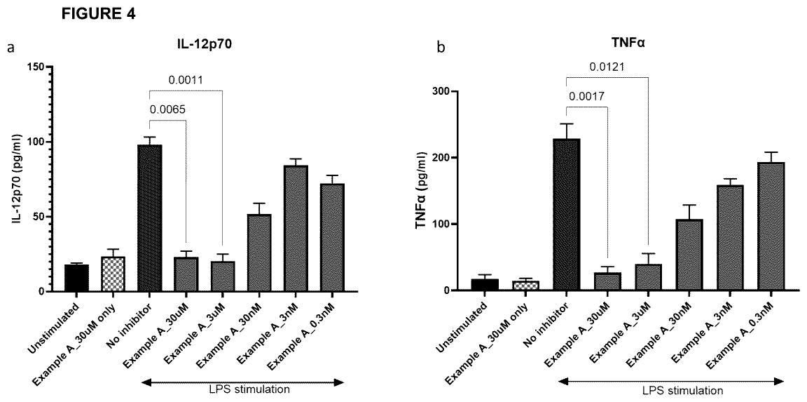

FIG. 5 shows the efficacy of Example A in an experimental autoimmune encephalomyelitis (EAE) mouse model.

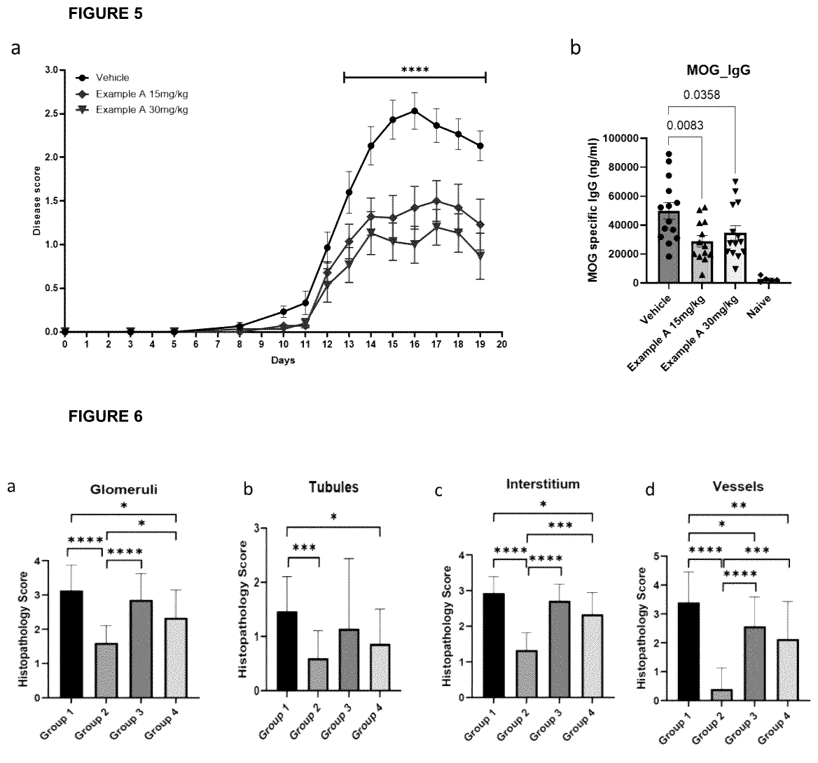

FIG. 6 shows the efficacy of Example A in the systemic lupus (SLE) disease model, kidney pathology.

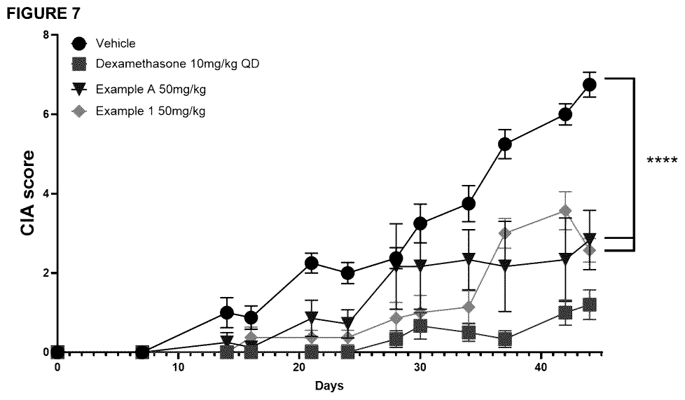

FIG. 7 shows the treatment with Example 1 and Example A significantly reduced clinical signs of collagen-induced arthritis (CIA).

### DETAILED DESCRIPTION

Provided herein are compounds of Formula I, or pharmaceutically acceptable salts thereof, that are useful as signal peptide peptidase like protease 2a (SPPL2a) inhibitors. These SPPL2a inhibitors are therefore useful in the treatment of various diseases and disorders associated with SPPL2a activity.

SPPL2a is the protease that cleaves the membrane-spanning portion of TNF-α. Because TNF-α is an inflammatory cytokine, targeting SPPL2a can treat inflammation (C. Spitz et al., Non-canonical Shedding of TNFα by SPPL2a Is Determined by the Conformational Flexibility of Its Transmembrane Helix. iScience. (2020) November 5; 23 (12): 101775). SPPL2a−/−mice exhibit impaired B cell maturation. Given the role of B cells in several autoimmune disorders such as rheumatoid arthritis, Sjogren's syndrome, multiple sclerosis, systemic lupus erythematosus (SLE), myasthenia gravis, and pemphigus vulgaris, it is suggested that inhibition of SPPL2a activity should be evaluated as a potential therapeutic strategy to treat autoimmune disorders (J. Schneppenheim et al., 2013). Depletion of B cells has been shown to be beneficial in a variety of autoimmune disorders including pemphigus vulgaris, Sjogren's disease, systemic lupus erythematosus (SLE), arthritis, lupus nephritis, neurological autoimmune diseases, autoantibody-mediated encephalitis syndromes, NMDAR encephalitis, Neuromyelitis Optica Spectrum Disorder (NMOSD), myelin-oligodendrocyte glycoprotein (MOG) spectrum disorder (MOGSD), multiple sclerosis, pemphigus foliaceus, myasthenia gravis, myocardial disorders, arthritis, rheumatoid arthritis, idiopathic thrombocytopenia purpura, Spondyloarthritis (SpA), vasculitis, and multiple myeloma (Mentrup T, et al. A Cell-Based Assay Reveals Nuclear Translocation of Intracellular Domains Released by SPPL Proteases. Traffic. 2015 August; 16 (8): 871-92 and Lee, D. S. W. et al. B cell depletion therapies in autoimmune disease: advances and mechanistic insights. Nat Rev Drug Discov (2021) 20, pages 179-199). For instance, SPPL2a inhibition in dendritic cells (DCs) disrupts TNFα cleavage leading to suppressed IL 12 and downstream IFNγ responses. In addition, build-up of CD74 in DCs may also disrupt their function particularly around providing cognate help to pathogenic T cells

A genome-wide study of psoriasis and psoriatic arthritis identified a disease locus harboring the gene for SPPL2a, suggesting novel drug targets for these diseases (Liu Y, et al. A genome-wide association study of psoriasis and psoriatic arthritis identifies new disease loci. PLoS Genet. 2008 Mar. 28). SPPL2a was expressed higher in a transcriptomic study of systemic juvenile idiopathic arthritis patient samples (Cepika A M, et al. A multidimensional blood stimulation assay reveals immune alterations underlying systemic juvenile idiopathic arthritis. J Exp Med. (2017) November 6; 214 (11): pages 3449-3466).

Given the role of SPPL2a in adaptive immunity and its cross presentation in regulating DCs, cytokines that regulate DCs (such as TNFα and IL-12, B-cell functions, and autoantibody production), SPPL2a inhibition could be therapeutically beneficial in treating various other inflammatory conditions, where antigen presentation, autoantibody production are crucial, along with substantial contribution of innate immune cells (APCs) and B-cells in the pathology of the diseases. Such diseases include hidradenitis suppurativa (Lowe et al., JCI Insight. 2020 Oct. 2; Byrd et al., Sci Transl Med. 2019 Sep. 4; Thomi et al., Br J Dermatol. 2017 November; 177 (5): 1358-1366), pemphigus disorders (Wang et al., J Invest Dermatol. 2024 October; Ramadan et al., Ann Dermatol. 2019 August; 31 (4): 454-457; Mashikilleyson et al., Acta Derm Venereol. 1989; 69 (5): 424-8; Ettinger et al, Autoimmunity. 2017 February; 50 (1): 25-36), ANCA vasculitis indications (Wilde et al., Nephrol Dial Transplant. 2009 July; 24 (7); Wilde et al. Kidney Int. 2011 March; 79 (6): 599-612; Braudeau C et al., Immunol. 2017 Feb. 9; 8:102), metabolic diseases such as diabetes (Guerder et al., Curr Opin Immunol. 2013 December; 25 (6): 670-5; Soedono et al., Int J Mol Sci. 2021 Aug. 12; 22 (16): 8666; Hotta-Iwamura et al., J Leukoc Biol. 2016 July; 100 (1): 65-80), insulin resistance (Bertola A. et al., Immunol. 2017 Feb. 9; 8:102), fatty liver disease (Deczkowska et al., Nat Med. 2021 June; Zhou et al., Front Immunol. 2022 Aug. 10), gall stone disease (Maurer K J et al., Gastroenterology, 2009 February; 136 (2): 425-40); and obesity (Sundara R. et al., Immunology. 2016 December; 149 (4): 353-361, Kae Won Cho et al., J Immunol 1 Nov. 2016; 197 (9): 3650-3661), temporal arteritis (Reitsema R D, et al., Front Immunol. 2023 Aug. 2; 14; Graver et al. Front Immunol. 2019 Jan. 29; 10:83), alopecia areata (Ito T. et al., Allergol Int. 2020 January; 69 (1): 121-131; Raghunandan et al., Genomics, Volume 96, Issue 3, 2010), primary biliary cirrhosis (Taylor S., et al., Semin Liver Dis. 2019 November; 39 (4): 422-431; Bjorkland et al., J Immunol 15 Sep. 1994; 153 (6): 2750-2757), autoimmune hemolytic anemia (Watanabe N., et al., 2002; Efremov D et al., Leuk Lymphoma. 1998 January; 28 (3-4): 285-93; Richards A, et al., Transfusion. 2021 January; 61 (1): 225-235), hyperlipidemia (Ito et al., Immunity. 2016), hypertension (Guzik et al., Nat Rev Cardiol. 2024 June; 21 (6): 396-416; Dixon K et al., Am J Physiol Heart Circ Physiol. 2017 Mar. 1; 312 (3)) and stroke (Gelderblom et al., Stroke. 2018 January; Hammond M et al., Transl Stroke Res. 2012 July; 3; Reichmann G et al., J Neuroimmunol. 2002 August).

As such, SPPL2a is a promising target for treating a variety of disorders.

### Definitions

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

At various places in the present specification, variables defining divalent linking groups may be described. These groups may be described explicitly with dashes or implicitly based on the nature of the disclosure (e.g., C₆₋₁₀ aryl-C₁₋₆ alkylene). Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted,” unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms. The phrase “substituted with one or more” is understood to only allow for as many substituents as valency permits. In an embodiment, “one or more” refers to 1 to 6 substituents, 1 to 5 substituents, 1 to 4 substituents, 1 to 3 substituents, 1 or 2 substituents, or 1 substituent.

The term “Cn-m” indicates a range which includes the endpoints, wherein n and m are integers and indicate the number of carbons. Examples include C₁₋₄, C₁₋₆ and the like.

The term “alkyl,” as used herein, refers to a fully saturated branched or straight chain hydrocarbon having up to 20 carbon atoms. In certain embodiments an alkyl group is a “C₁-C₂alkyl,” “C₁-C₃alkyl,” “C₁-C₆alkyl,” “C₁-C₆alkyl,” “C₁-C₆alkyl,” “C₁-C₂alkyl,” “C₁-C₆alkyl,” “C₁-C₆alkyl,” or “C₁-C₁₀alkyl” wherein the terms “C₁-C₂alkyl” “C₁-C₃alkyl,” “C₁-C₄alkyl,” “C₁-C₅alkyl,” “C₁-C₆alkyl,” “C₁-C₂alkyl,” “C₁-C₈alkyl,” “C₁-C₆alkyl” and “C₁-C₁₀alkyl,” as used herein, refer to an alkyl group containing at least 1, and at most 2, 3, 4, 5, 6, 7, 8, 9 or 10 carbon atoms, respectively. Non-limiting examples of alkyl groups include methyl, ethyl, n-propyl, isopropyl, n-butyl, isobutyl, sec-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl and n-decyl.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” refers to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “alkylene,” employed alone or in combination with other terms, refers to a divalent alkyl linking group. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

The term “alkoxy,” as used herein, refers to —O-alkyl or -alkyl-O—, wherein “alkyl” is as defined herein. In certain embodiments an alkoxy group is a “C₁-C₂alkoxy,” “C₁-C₃alkoxy,” “C₁-C₆alkoxy,” “C₁-C₆alkoxy,” “C₁-C₆alkoxy,” “C₁-C₂alkoxy,” “C₁-C₈alkoxy,” “C₁-C₆alkoxy” or “C₁-C₁₀alkoxy,” wherein the terms “C₁-C₃alkoxy,” “C₁-C₄alkoxy,” “C₁-C₆alkoxy,” “C₁-C₆alkoxy,” “C₁-C₂alkoxy,” “C₁-C₈alkoxy,” “C₁-C₆alkoxy” and “C₁-C₁₀alkoxy,” as used herein refer to —O—C₁-C₂alkyl, —O—C₁-C₃alkyl, —O—C₁-C₆alkyl, —O—C₁-C₆alkyl, —O—C₁-C₆alkyl, —O—C₁-C₂alkyl, —O—C₁-C₆alkyl, —O—C₁-C₆alkyl or —O—C₁-C₁₀alkyl, respectively. Non-limiting examples of “alkoxy” groups include methoxy, ethoxy, n-propoxy, isopropoxy, n-butoxy, isobutoxy, sec-butoxy, tert-butoxy, n-pentoxy, isopentoxy, hexoxy, heptoxy, octoxy, nonoxy and decoxy.

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. Aryl groups include, e.g., phenyl, naphthyl, and the like. In some embodiments, aryl groups have from 6 to about 10 carbon atoms. In some embodiments, aryl groups have 6 carbon atoms. In some embodiments, aryl groups have 10 carbon atoms. In some embodiments, the aryl group is phenyl. In some embodiments, the aryl group is naphthyl.

The term “cycloalkyl” as used herein, refers to a fully saturated, monocyclic hydrocarbon ring system having carbon atoms as ring members. Non-limiting examples of such “C₃-C₈cycloalkyl” groups include cyclopropyl, cyclobutyl, cyclopentyl, cyclohexyl, cycloheptyl and cyclooctyl. In certain embodiments, the term “C₃-C₆cycloalkyl” as used herein, refers to a fully saturated, monocyclic hydrocarbon ring system having 3 to 6 carbon atoms as ring members. Non-limiting examples of such “C₃-C₆cycloalkyl” groups include cyclopropyl, cyclobutyl, cyclopentyl and cyclohexyl.

The term “carbocyclic ring,” as used herein, refers to a saturated or partially saturated hydrocarbon ring. Non-limiting examples of such carbocyclic ring groups include cyclopropyl, cyclobutyl, cyclopentyl, cyclohexyl, cycloheptyl, cyclooctyl, cyclopentenyl, cyclohexenyl, cycloheptenyl and cyclooctenyl.

The term “cycloalkenyl” as used herein, refers to a partially saturated (but not aromatic), monocyclic hydrocarbon ring system having carbon atoms as ring members.

The term “C₁-C₆alkyl-phenyl” as used herein, refer to a C₁-C₆alkyl as defined above which is substituted with a phenyl group. Non-limiting example of a C₁-C₆alkyl-phenyl is benzyl.

The term “haloalkyl” as used herein, refers to an alkyl group as defined herein, wherein at least one of the hydrogen atoms of the alkyl is replaced by a halo group (as defined herein). The haloalkyl can be monohaloalkyl, dihaloalkyl, trihaloalkyl, or polyhaloalkyl including perhaloalkyl. A monohaloalkyl can have one iodo, bromo, chloro or fluoro within the alkyl group. Dihaloalkyl and polyhaloalkyl groups can have two or more of the same halo atoms or a combination of different halo groups within the alkyl. Typically, the polyhaloalkyl contains up to 6, or 4, or 3, or 2 halo groups. Non-limiting examples of haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-alkyl refers to an alkyl having all hydrogen atoms replaced with halo atoms, e.g., trifluoromethyl. Preferred haloalkyl groups, unless specified otherwise, include monofluoro-, difluoro- and trifluoro-substituted methyl and ethyl groups, e.g., CF₃, CHF₂, CH₂F, CH₂CHF₂ and CH₂CF₃.

The term “C₁-C₆haloalkyl” as used herein, refers to the respective “C₁-C₆alkyl,” as defined herein, wherein at least one of the hydrogen atoms of the “C₁-C₆alkyl” is replaced by a halo group (as defined herein). The C₁-C₆haloalkyl groups can be monoC₁-C₆haloalkyl, wherein such C₁-C₆haloalkyl groups have one iodo, one bromo, one chloro or one fluoro. Additionally, the C₁-C₆haloalkyl groups can be diC₁-C₆haloalkyl wherein such C₁-C₆haloalkyl groups can have two halo atoms independently selected from iodo, bromo, chloro or fluoro. Furthermore, the C₁-C₆haloalkyl groups can be polyC₁-C₆haloalkyl wherein such C₁-C₆haloalkyl groups can have two or more of the same halo atoms or a combination of two or more different halo atoms. Such polyC₁-C₆haloalkyl can be perhaloC₁-C₆haloalkyl where all the hydrogen atoms of the respective C₁-C₆alkyl have been replaced with halo atoms and the halo atoms can be the same or a combination of different halo atoms. Non-limiting examples of “C₁-C₆haloalkyl” groups include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, fluoroethyl, difluoroethyl, trifluoroethyl, difluoropropyl, dichlorocthyl and dichloropropyl.

The term “haloalkoxy” as used herein, refers to the group-O-haloalkyl wherein at least one of the hydrogen atoms of the alkyl group of the alkoxy is replaced by a halo group (as defined herein). The haloalkoxy can be monohaloalkoxy, dihaloalkoxy, trihaloalkoxy, or polyhaloalkoxy including perhaloalkoxy. A monohaloalkoxy can have one iodo, bromo, chloro or fluoro within the alkyl group. Dihaloalkoxy and polyhaloalkoxy groups can have two or more of the same halo atoms or a combination of different halo groups within the alkyl. Typically, the polyhaloalkoxy contains up to 6, or 4, or 3, or 2 halo groups. Non-limiting examples of haloalkoxy include fluoromethoxy, difluoromethoxy, trifluoromethoxy, chloromethoxy, dichloromethoxy, trichloromethoxy, pentafluoroethoxy, heptafluoropropoxy, difluorochloromethoxy, dichlorofluoromethoxy, difluoroethoxy, difluoropropoxy, dichloroethoxy and dichloropropoxy. A perhalo-alkoxy refers to an alkoxy having all hydrogen atoms replaced with halo atoms, e.g., trifluoromethoxy. Preferred haloalkoxy groups, unless specified otherwise, include monofluoro-, difluoro- and trifluoro-substituted methoxy and ethoxygroups, e.g., —OCF₃, —OCHF₂, —OCH₂F, —OCH₂CHF₂ and —OCH₂CF₃.

The term “C₁-C₆haloalkoxy” as used herein, refers to the group-O—C₁-C₆haloalkyl, wherein at least one of the hydrogen atoms of the “C₁-C₆alkyl” of the “C₁-C₆alkoxy” is replaced by a halo group (as defined herein). The C₁-C₆haloalkoxy groups can be monoC₁-C₆haloalkoxy, wherein such C₁-C₆haloalkoxy groups have one iodo, one bromo, one chloro or one fluoro. Additionally, the C₁-C₆haloalkoxy groups can be diC₁-C₆haloalkoxy wherein such C₁-C₆haloalkoxy groups can have two halo atoms independently selected from iodo, bromo, chloro or fluoro. Furthermore, the C₁-C₆haloalkoxy groups can be polyC₁-C₆haloalkoxy wherein such C₁-C₆haloalkoxy groups can have two or more of the same halo atoms or a combination of two or more different halo atoms. Such polyC₁-C₆haloalkoxy can be perhaloC₁-C₆haloalkoxy where all the hydrogen atoms of the respective C₁-C₆alkoxy have been replaced with halo atoms and the halo atoms can be the same or a combination of different halo atoms. Non-limiting examples of “C₁-C₆haloalkoxy” groups include fluoromethoxy, difluoromethoxy, trifluoromethoxy, chloromethoxy, dichloromethoxy, trichloromethoxy, pentafluoroethoxy, heptafluoropropoxy, difluorochloromethoxy, dichlorofluoromethoxy, fluoroethoxy, difluoroethoxy, trifluoroethoxy, difluoropropoxy, dichloroethoxy and dichloropropoxy.

The terms “halogen” or “halo” as used herein, refer to fluoro (F), chloro (Cl), bromo (Br) and iodo (I).

The term “heteroatoms” or “hetero atoms” as used herein, refer to nitrogen (N), oxygen (O) or sulfur(S) atoms.

The term “heteroaryl,” as used herein, refers to an aromatic ring system containing one or more heteroatoms. Heteroaryl groups containing more than one heteroatom may contain different heteroatoms. Heteroaryl groups may be monocyclic ring systems or fused bicyclic ring systems. Monocyclic heteroaryl rings have from 5 to 6 ring atoms. Bicyclic heteroaryl rings have from 7 to 12 ring member atoms. Bicyclic heteroaryl rings include those ring systems wherein a heteroaryl ring is fused to a phenyl ring. Non-limiting examples of heteroaryl groups, as used herein, include benzofuranyl, benzo[c]thiophenyl, benzothiophenyl, benzoxazolyl, benzothiazolyl, benzimidazolyl, cinnolinyl, furazanyl, furyl, imidazolyl, indolyl, indolizinyl, indazolyl, isoindolyl, isoquinolinyl, isoxazolyl, isothiazolyl, oxazolyl, oxaindolyl, oxadiazolyl (including 1,3,4-oxadiazolyl and 1,2,4-oxadiazolyl), purinyl, pyrazolyl, pyrrolyl, phthalazinyl, pyridinyl (including 2-, 3-, and 4-pyridinyl), pyridazinyl, pyrazinyl, pyrimidinyl, quinoxalinyl, quinolinyl, quinazolinyl, tetrazinyl, tetrazolyl, tetrazolo[1,5-a]pyridinyl, thiazolyl, thiadiazolyl (including 1,3,4-thiadiazolyl), thicnyl, triazinyl, and triazolyl.

The term “5-membered heteroaryl” as used herein, refers to an aromatic, 5 membered monocyclic ring system having 1, 2 or 3 heteroatoms as ring members, each of which is independently selected from N, O and S. Non-limiting examples of such 5 membered heteroaryl groups, as used herein, include furyl, imidazolyl, isoxazolyl, isothiazolyl, oxazolyl, pyrrolyl, pyrazolyl, thiadiazolyl, thiazolyl, thienyl and triazolyl. In certain embodiments the “5-membered heteroaryl,” as used herein, refers to an aromatic, 5 membered monocyclic ring system having 1 or 2 heteroatoms as ring members, each of which is independently selected from N, O and S. Non-limiting examples of such 5 membered heteroaryl groups, as used herein, include furyl, imidazolyl, isoxazolyl, isothiazolyl, oxazolyl, pyrrolyl, pyrazolyl, thiadiazolyl, thiazolyl, thienyl and triazolyl.

The term “6-membered heteroaryl” as used herein, refers to an aromatic, 6 membered monocyclic ring system having 1, 2 or 3 heteroatoms as ring members, each of which is independently selected from N, O and S. Non-limiting examples of such 6 membered heteroaryl groups, as used herein, include pyridyl, pyridazinyl, pyrazinyl, pyrimidinyl and triazinyl. In certain embodiments the term “6-membered heteroaryl,” as used herein, refers to an aromatic, 6 membered monocyclic ring system having 1 or 2 heteroatoms as ring members, each of which is independently selected from N, O and S. Non-limiting examples of such 6 membered heteroaryl groups, as used herein, include pyridyl, pyridazinyl, pyrazinyl, and pyrimidinyl.

The term “9- or 10-membered bicyclic heteroaryl” as used herein, refers to a 9 or 10 membered fused, bicyclic aromatic ring system having 1, 2, 3 or 4 heteroatoms as ring members, each of which is independently selected from N, O and S. Non-limiting examples of such bicyclic heteroaryl groups, as used herein, include indolyl, quinolinyl, isoquinolinyl, indazolyl, purinyl, phthalazinyl, naphthyridinyl, quinazolinyl, cinnolinyl, thieno[2,3-b]furanyl, 1H-pyrazolo[4,3-d]-oxazolyl, imidazo[2,1-b]thiazolyl, pyrazino[2,3-d]pyridazinyl, imidazo[1,2-b][1,2,4]triazinyl, benzoxazolyl, benzimidazolyl, imidazopyridinyl and benzothiazolyl. In certain embodiments such a bicyclic heteroaryl group is 1H-benzo[d]imidazolyl or 1H-imidazo[4,5-c]pyridinyl.

The term “heterocyclyl” as used herein, refers to saturated or partially saturated hydrocarbon ring containing 1 to 2 heteroatoms as ring members, each independently selected from N, NH, NR^{A}, O or S, where R^{A} is H, C₁-C₆alkyl or C₃-C₈cycloalkyl. The heterocyclyl group can be attached to another group at a nitrogen or a carbon atom. Non-limiting examples of heterocycloalkyl groups, as used herein, include azetadinyl, azetadin-1-yl, azetadin-2-yl, azetadin-3-yl, oxetanyl, oxetan-2-yl, oxetan-3-yl, oxetan-4-yl, thietanyl, thietan-2-yl, thietan-3-yl, thietan-4-yl, pyrrolidinyl, pyrrolidin-1-yl, pyrrolidin-2-yl, pyrrolidin-3-yl, pyrrolidin-4-yl, pyrrolidin-5-yl, tetrahydrofuranyl, tetrahydrofuran-2-yl, tetrahydrofuran-3-yl, tetrahydrofuran-4-yl, tetrahydrofuran-5-yl, tetrahydrothienyl, tetrahydrothien-2-yl, tetrahydrothien-3-yl, tetrahydrothien-4-yl, tetrahydrothien-5-yl, piperidinyl, piperidin-1-yl, piperidin-2-yl, piperidin-3-yl, piperidin-4-yl, piperidin-5-yl, piperidin-6-yl, tetrahydropyranyl, tetrahydropyran-2-yl, tetrahydropyran-3-yl, tetrahydropyran-4-yl, tetrahydropyran-5-yl, tetrahydropyran-6-yl, tetrahydrothiopyranyl, tetrahydrothiopyran-2-yl, tetrahydrothiopyran-3-yl, tetrahydrothiopyran-4-yl, tetrahydrothiopyran-5-yl, tetrahydrothiopyran-6-yl, piperazinyl, piperazin-1-yl, piperazin-2-yl, piperazin-3-yl, piperazin-4-yl, piperazin-5-yl, piperazin-6-yl, morpholinyl, morpholin-2-yl, morpholin-3-yl, morpholin-4-yl, morpholin-5-yl, morpholin-6-yl, thiomorpholinyl, thiomorpholin-2-yl, thiomorpholin-3-yl, thiomorpholin-4-yl, thiomorpholin-5-yl, thiomorpholin-6-yl, oxathianyl, oxathian-2-yl, oxathian-3-yl, oxathian-5-yl, oxathian-6-yl, dithianyl, dithian-2-yl, dithian-3-yl, dithian-5-yl, dithian-6-yl, dioxolanyl, dioxolan-2-yl, dioxolan-4-yl, dioxolan-5-yl, thioxanyl, thioxan-2-yl, thioxan-3-yl, thioxan-4-yl, thioxan-5-yl, dithiolanyl, dithiolan-2-yl, dithiolan-4-yl, dithiolan-5-yl, pyrazolidinyl, pyrazolidin-1-yl, pyrazolidin-2-yl, pyrazolidin-3-yl, pyrazolidin-4-yl, pyrazolidin-5-yl, 2-azabicyclo[4.2.0]octanyl, octahydro-1H-cyclopenta[b]pyridine and decahydroquinoline.

The term “isomers” as used herein, refers to different compounds that have the same molecular formula but differ in arrangement and configuration of the atoms. Also as used herein, the term “an optical isomer” or “a stereoisomer” refers to any of the various stereo isomeric configurations which may exist for a given compound of the present disclosure and includes geometric isomers. It is understood that a substituent may be attached at a chiral center of a carbon atom. The term “chiral” refers to molecules which have the property of non-superimposability on their mirror image partner, while the term “achiral” refers to molecules which are superimposable on their mirror image partner. Therefore, the disclosure includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain compounds described herein contain one or more asymmetric centers or axes and may thus give rise to enantiomers, diastereomers, and other stereoisomeric forms that may be defined, in terms of absolute stereochemistry, as (R)- or (S)-.

The term “pharmaceutically acceptable carrier” as used herein, includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The phrase “a therapeutically effective amount” of a compound of the present disclosure refers to an amount of the compound of the present disclosure that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present disclosure that, when administered to a subject, is effective to (1) at least partially alleviating, inhibiting, preventing and/or amcliorating a condition, or a disorder or a disease (i) mediated by SPPL2a, or (ii) associated with or mediated by SPPL2a activity, or (iii) characterized by activity (normal or abnormal) of SPPL2a; or (2) reducing or inhibiting the activity of SPPL2a; or (3) reducing or inhibiting the expression of SPPL2a. In another non-limiting embodiment, the term “a therapeutically effective amount” refers to the amount of the compound of the present disclosure that, when administered to a cell, or a tissue, or a non-cellular biological material, or a medium, is effective to at least partially reducing or inhibiting the activity of SPPL2a; or at least partially reducing or inhibiting the expression of SPPL2a.

The term “subject” as used herein may refer to an animal. The animal may be a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

The terms “inhibit,” “inhibition,” or “inhibiting,” as used herein, refers to the reduction or suppression of a given condition, symptom, or disorder, or disease, or a significant decrease in the baseline activity of a biological activity or process.

As used herein, the terms “treat,” “treating” or “treatment” of any disease or disorder refer in one embodiment, to ameliorating the disease or disorder (i.e., slowing or arresting or reducing the development of the disease or at least one of the clinical symptoms thereof). In another embodiment “treat,” “treating” or “treatment” refers to alleviating or ameliorating at least one physical parameter including those which may not be discernible by the patient. In yet another embodiment, “treat,” “treating” or “treatment” refers to modulating the disease or disorder, either physically, (e.g., stabilization of a discernible symptom), physiologically, (e.g., stabilization of a physical parameter), or both.

As used herein, the term “preventing” refer to delaying the onset or development or progression of the disease or disorder.

As used herein, a subject is “in need of′ a treatment if such subject would benefit biologically, medically or in quality of life from such treatment.

As used herein, the term “a,” “an,” “the” and similar terms used in the context of the present disclosure (especially in the context of the claims) are to be construed to cover both the singular and plural unless otherwise indicated herein or clearly contradicted by the context.

Various enumerated embodiments of the present disclosure are described herein. It will be recognized that features specified in each embodiment may be combined with other specified features to provide further embodiments of the present disclosure.

### Compounds

Provided herein is a compound of Formula (I), or a pharmaceutical acceptable salt or stereoisomer thereof,

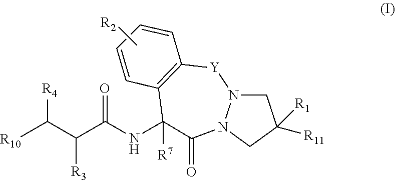

wherein:
Y is CH₂ or C═O;
R₁ is H, C₁-C₆alkyl or halo;
R₂ is H, halo, C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₄ is H, C₁-C₆alkyl or C₁-C₆alkyl-phenyl;
R₁₀ is —NHC(═O)R₅, —C(═O)NHR₅ or a 9 or 10 membered bicyclic heteroaryl having 1 to 4 heteroatoms as ring members each independently selected from N, O and S, wherein the bicyclic heteroaryl is unsubstituted or the bicyclic heteroaryl is substituted with one or more R₆;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by one or more D or C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo;
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy; and
xxi) —C(═O)NH₂;
each R₆ is independently selected from C₁-C₆alkyl, C₁-C₆alkoxy, C₁-C₆haloalkyl, cyano and halo; and
R₁₁ is H, C₁-C₆alkyl or halo; or
R₁ and R₁₁ together with the carbon atom to which they are attached, may form a 3 to 6 membered carbocyclic ring;
R⁷ is selected from D, CD₃, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, (4-10 membered heterocycloalkyl)-C₁₋₆ alkylene, CN, C(O)R^{b7}, C(O)NR^{c7}R^{d7}, C(O)NR^{c7}(ORd^{d7}), C(O)OR^{d7}, C(═NR^{e7})R^{b7}, C(═NR^{e7})NR^{c7}R^{d7}, S(O)R^{b7}, S(O)NR^{c7}R^{d7}, S(O)₂R^{b7}, and S(O)₂NR^{c7}R^{d7}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R⁷ are each optionally substituted with 1, 2, 3, 4, 5, 6, 7, or 8 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R^{a7}, R^{c7}, and R^{d7} are each optionally substituted with 1, 2, 3, 4, 5, 6, 7, or 8 independently selected R^{7A} substituents;
or any R^{c7} and R^{d7} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{7A} substituents;
each R^{b7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R^{b7} are each optionally substituted with 1, 2, 3, 4, 5, 6, 7, or 8 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene;
R^{7A} is selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, (4-7 membered heterocycloalkyl)-C₁₋₆ alkylene, CN, NO₂, OR^{a71}, SR^{a71}, NHOR^{a71}, C(O)R^{b71}, C(O)NR^{e71}R^{d71}, C(O)NR^{c71} (OR^{a71}), C(O)OR^{d71}, OC(O)R^{b71}, OC(O)NR^{e71}R^{d71}, NR^{e71}R^{d71}, NR^{c71}NR^{c71}R^{d71} NR^{c71}C(O)R^{b71}, NR^{e71}C(O)OR^{d71}, NR^{c71}C(O)NR^{c71}R^{d71}, C(═NR^{e71})R^{b71}, C(═NR^{e71})NR^{e71}R^{d71}, NR^{c71}C(═NR^{e71})NR^{c71}R^{d71}, NR^{c71}C(═NR^{e71})R^{b71}, NR^{e71}S(O)R^{b71}, NR^{e71}S(O)NR^{e71}R^{d71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, and OS(O)₂R^{b71}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene of R^{7A} are each optionally substituted with 1, 2, 3, or 4 independently selected R^{M} substituents;
each R^{7A} is selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, (4-7 membered heterocycloalkyl)-C₁₋₆ alkylene, CN, NO₂, OR^{a71}, SR^{a71}, NHOR^{a71}, C(O)R^{b71}, C(O)NR^{e71}R^{d71}, C(O)NR^{e71} (OR^{a71}), C(O)OR^{d71}, OC(O)R^{b71}, OC(O)NR^{c71}R^{d71}, NR^{e71}R^{d71}, NR^{c71}NR^{c71}R^{d71} NR^{a71}C(O)R^{b71}, NR^{c71}C(O)OR^{d71}, NR^{c71}C(O)NR^{c71}R^{d71}, C(═NR^{e71})R^{b71}, C(═NR^{e71})NR^{c71}R^{d71} NR^{c71}C(═NR^{e71})NR^{e71}R^{d71}, NR^{e71}C(═NR^{e71})R^{b71}, NR^{c71}S(O)R^{b71}, NR^{c71}S(O)NR^{c71}R^{d71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71} S(O)₂R^{b71}, S(O)₂NR^{c71}R^{d71}, OS(O)(═NR^{e71})R^{b71}, and OS(O)₂R^{b71}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{7A} are each optionally substituted with 1, 2, 3, or 4 independently selected R^{M} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{a71}, R^{c71}, and R^{d71} are each optionally substituted with 1, 2, 3, or 4 independently selected R^{M} substituents;
or any Roll and R^{d71} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b71} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{b71} are each optionally substituted with 1, 2, 3, or 4 independently selected R^{M} substituents;
each R^{e71} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene; and each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene.

In an embodiment,
Y is CH₂ or C═O;
R₁ is H, C₁-C₆alkyl or halo;
R₂ is H, halo, C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₄ is H, C₁-C₆alkyl or C₁-C₆alkyl-phenyl;
R₁₀ is —NHC(═O)R₅, —C(═O)NHR₅ or a 9 or 10 membered bicyclic heteroaryl having 1 to 4 heteroatoms as ring members each independently selected from N, O and S, wherein the bicyclic heteroaryl is unsubstituted or the bicyclic heteroaryl is substituted with one or more R₆;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo; and
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy;
each R₆ is independently selected from C₁-C₆alkyl, C₁-C₆alkoxy, C₁-C₆haloalkyl, cyano and halo;
R₁₁ is H, C₁-C₆alkyl or halo; or
R₁ and R₁₁ together with the carbon atom to which they are attached, may form a 3 to 6 membered carbocyclic ring;
R⁷ is selected from D, CD₃, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, (4-10 membered heterocycloalkyl)-C₁₋₆ alkylene, CN, C(O)R^{b7}, C(O)NR^{c7}R^{d7}, C(O)NR^{c7}(OR^{a7}), C(O)OR^{d7}, C(═NR^{e7})R^{b7}, C(═NR^{e7})NR^{c7}R^{d7}, S(O)R^{b7}, S(O)NR^{c7}R^{d7}, S(O)₂R^{b7}, and S(O)₂NR^{c7}R^{d7}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R⁷ are each optionally substituted with 1, 2, 3, 4, 5, 6, 7, or 8 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R^{a7}, R^{c7}, and R^{d7} are each optionally substituted with 1, 2, 3, 4, 5, 6, 7, or 8 independently selected R^{7A} substituents;
or any R^{c7} and R^{d7} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{7A} substituents;
each R^{b7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, and (4-10 membered heterocycloalkyl)-C₁₋₆ alkylene, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, and (4-10 membered heterocycloalkyl)-C₁₋₆ alkylene of R^{b7} are each optionally substituted with 1, 2, 3, 4, 5, 6, 7, or 8 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene;
R^{7A} is selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, (4-7 membered heterocycloalkyl)-C₁₋₆ alkylene, CN, NO₂, OR^{a71}, SR^{a71}, NHOR^{a71}, C(O)R^{b71}, C(O)NR^{e71}R^{d71}, C(O)NR^{c71} (OR^{a71}), C(O)OR^{d71}, OC(O)R^{b71}, OC(O)NR^{c71}R^{d71}, NR^{e71}R^{d71}, NR^{c71}NR^{c71}R^{d71} NR⁷¹C(O)R^{b71}, NR^{e71}C(O)OR^{d71}, NR^{e71}C(O)NR^{c71}R^{d71}, C(═NR^{e71})R^{b71}, C(═NR^{e71})NR^{e71}R^{d71} NR^{c71}C(═NR^{e71})NR^{c71}R^{d71}, NR⁷¹C(═NR^{e71})R^{b71}, NR^{e71}S(O)R^{b71}, NR^{e71}S(O)NR^{e71}R^{d71}, NR^{c71}S(O)₂R^{b71}, NR^{c71}S(O)(═NR^{e71})R^{b71}, NR^{c71}S(O)₂NR^{e71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, and OS(O)₂R^{b71}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene of R^{7A} are each optionally substituted with 1, 2, 3, or 4 independently selected R^{M} substituents;
each R^{7A} is selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, (4-7 membered heterocycloalkyl)-C₁₋₆ alkylene, CN, NO₂, OR^{a71}, SR^{a71}, NHOR^{a71}, C(O)R^{b71}, C(O)NR^{e71}R^{d71} C(O)NR^{e71} (OR^{a71}), C(O)OR^{d71}, OC(O)R^{b71}, OC(O)NR^{c71}R^{d71}, NR^{e71}R^{d71}, NRCZINR^{e71}R^{d71} NR⁷¹C(O)R^{b71}, NR^{c71}C(O)OR^{d71}, NR^{c71}C(O)NR^{c71}R^{d71}, C(═NR^{e71})R^{b71}, C(═NR^{e71})NR^{c71}R^{d71} NR^{c71}C(═NR^{e71})NR^{c71}R^{d71}, NR^{c71}C(═NR^{e71})R^{b71}, NR^{c71}S(O)R^{b71}, NR^{c71}S(O)NR^{e71}R^{d71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, and OS(O)₂R^{b71}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{7A} are each optionally substituted with 1, 2, 3, or 4 independently selected R^{M} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{a71}, R^{c71}, and R^{d71} are each optionally substituted with 1, 2, 3, or 4 independently selected R^{M} substituents;
or any R^{c71} and R^{d71} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b71} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{b71} are each optionally substituted with 1, 2, 3, or 4 independently selected R^{M} substituents;
each R^{e71} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene; and each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene.

In another aspect, provided herein is a compound of Formula (Ia) or a pharmaceutically acceptable salt or stereoisomer thereof,

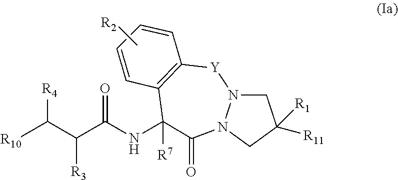

wherein:
Y is CH₂ or C═O;
R₁ is H, C₁-C₆alkyl or halo;
R₂ is C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₄ is H, C₁-C₆alkyl or C₁-C₆alkyl-phenyl;
R₁₀ is —NHC(═O)R₅, —C(═O)NHR, or a 9 or 10 membered bicyclic heteroaryl having 1 to 4 heteroatoms as ring members each independently selected from N, O and S, wherein the bicyclic heteroaryl is unsubstituted or the bicyclic heteroaryl is substituted with one or more R₆;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by one or more D or C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo;
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy; and
xxi) —C(═O)NH₂;
each R₆ is independently selected from C₁-C₆alkyl, C₁-C₆alkoxy, C₁-C₆haloalkyl, cyano and halo;
R₁₁ is H, C₁-C₆alkyl or halo; or
R₁ and R₁₁ together with the carbon atom to which they are attached, may form a 3 to 6 membered carbocyclic ring; and
R⁷ is H.

In an embodiment, R₂ is C₁-C₆alkyl. In another embodiment, R₂ is C₁-C₆haloalkyl. In yet another embodiment, R₂ is C₁-C₆alkyloxy. In still another embodiment, R₂ is C₁-C₆haloalkyloxy.

Various embodiments of the compounds of the present disclosure are described herein. It will be recognized that features specified in each embodiment may be combined with other specified features to provide further embodiments. The following enumerated embodiments are representative of the compounds of Formula (I) of the present disclosure.

Embodiment 1. A compound of Formula (I), or a pharmaceutically acceptable salt or stereoisomer thereof,

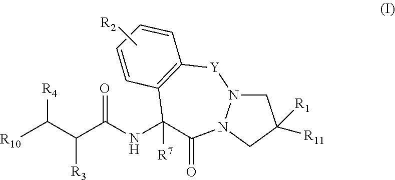

wherein:
Y is CH₂ or C═O;
R₁ is H, C₁-C₆alkyl or halo;
R₂ is H, halo, C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₄ is H, C₁-C₆alkyl or C₁-C₆alkyl-phenyl;
R₁₀ is —NHC(═O)R₅, —C(═O)NHR₅ or a 9 or 10 membered bicyclic heteroaryl having 1 to 4 heteroatoms as ring members each independently selected from N, O and S, wherein the bicyclic heteroaryl is unsubstituted or the bicyclic heteroaryl is substituted with one or more R₆;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by one or more D or C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo;
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy; and
xxi) —C(═O)NH₂;
each R₆ is independently selected from C₁-C₆alkyl, C₁-C₆alkoxy, C₁-C₆haloalkyl, cyano and halo;
R₁₁ is H, C₁-C₆alkyl or halo; or
R₁ and R₁₁ together with the carbon atom to which they are attached, may form a 3 to 6 membered carbocyclic ring;
R⁷ is selected from D, CD₃, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, (4-10 membered heterocycloalkyl)-C₁₋₆ alkylene, CN, C(O)R^{b7}, C(O)NR^{c7}R^{d7}, C(O)NR^{c7}(ORd^{d7}), C(O)OR^{d7}, C(═NR^{e7})R^{b7}, C(═NR^{e7})NR^{c7}R^{d7}, S(O)R^{b7}, S(O)NR^{c7}R^{d7}, S(O)₂R^{b7}, and S(O)₂NR^{c7}R^{d7}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R⁷ are each optionally substituted with 1, 2, 3, 4, 5, 6, 7, or 8 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R^{a7}, R^{e7}, and R^{d7} are each optionally substituted with 1, 2, 3, 4, 5, 6, 7, or 8 independently selected R^{7A} substituents;
or any R^{c7} and R^{d7} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{7A} substituents;
each R^{b7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, and (4-10 membered heterocycloalkyl)-C₁₋₆ alkylene, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, and (4-10 membered heterocycloalkyl)-C₁₋₆ alkylene of R^{b7} are each optionally substituted with 1, 2, 3, 4, 5, 6, 7, or 8 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene;
R^{7A} is selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, (4-7 membered heterocycloalkyl)-C₁₋₆ alkylene, CN, NO₂, OR^{a71}, SR^{a71}, NHOR^{a71}, C(O)R^{b71}, C(O)NR^{c71}R^{d71}, C(O)NR^{e71} (OR^{a71}), C(O)OR^{d71}, OC(O)R^{b71}, OC(O)NR^{e71}R^{d71}, NR^{e71}R^{d71}, NR^{c71}NR^{c71}R^{d71} NR^{c71}C(O)R^{b71}, NR^{c71}C(O)OR^{d71}, NR^{e71}C(O)NR^{c71}R^{d71}, C(═NR^{e71})R^{b71}, C(═NR^{e71})NR⁷¹R^{d71}, NR^{c71}C(═NR^{e71})NR^{c71}R^{d71}, NR^{e71}C(═NR^{e71})R^{b71}, NR^{c71}S(O)R^{b71}, NR^{c71}S(O)NR^{e71}R^{d71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, and OS(O)₂R^{b71}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene of R^{7A} are each optionally substituted with 1, 2, 3, or 4 independently selected R^{M} substituents;
each R^{7A} is selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, (4-7 membered heterocycloalkyl)-C₁₋₆ alkylene, CN, NO₂, OR^{a71}, SR^{a71}, NHOR^{a71}, C(O)R^{b71}, C(O)NR^{e71}R^{d71}, C(O)NR^{c71} (OR^{a71}), C(O)OR^{d71}, OC(O)R^{b71}, OC(O)NR^{c71}R^{d71}, NR^{e71}R^{d71}, NR^{c71}NR^{c71}R^{d71} NR^{c71}C(O)R^{b71}, NR^{c71}C(O)OR^{d71}, NR^{c71}C(O)NR^{c71}R^{d71}, C(═NR^{e71})R^{b71}, C(═NR^{e71})NR^{c71}R^{d71} NR^{c71}C(═NR^{e71})NR^{c71}R^{d71}, NR^{c71}C(═NR^{e71})R^{b71}, NR^{c71}S(O)R^{b71}, NR^{c71}S(O)NR^{c71}R^{d71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, and OS(O)₂R^{b71}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ Cycloalkyl-C₁₋₆ alkylene, (5-6 membered heteroaryl)-C₁₋₆ alkylene, and (4-7 membered heterocycloalkyl)-C₁₋₆ alkyl-of R^{7A} are each optionally substituted with 1, 2, 3, or 4 independently selected R^{M} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{a71}, R^{c71}, and R^{d71} are each optionally substituted with 1, 2, 3, or 4 independently selected R^{M} substituents;
or any R^{c71} and R^{d71} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b71} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{b71} are each optionally substituted with 1, 2, 3, or 4 independently selected R^{M} substituents;
each R^{e71} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene; and each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene.

Embodiment 1A: The compound of Embodiment 1, or a pharmaceutically acceptable salt or stereoisomer thereof, wherein
Y is CH₂ or C═O;
R₁ is H, C₁-C₆alkyl or halo;
R₂ is H, halo, C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₄ is H, C₁-C₆alkyl or C₁-C₆alkyl-phenyl;
R₁₀ is —NHC(═O)R₅, —C(═O)NHR, or a 9 or 10 membered bicyclic heteroaryl having 1 to 4 heteroatoms as ring members each independently selected from N, O and S, wherein the bicyclic heteroaryl is unsubstituted or the bicyclic heteroaryl is substituted with one or more R₆;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo; and
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy;
each R₆ is independently selected from C₁-C₆alkyl, C₁-C₆alkoxy, C₁-C₆haloalkyl, cyano and halo;
R₁₁ is H, C₁-C₆alkyl or halo; or
R₁ and R₁₁ together with the carbon atom to which they are attached, may form a 3 to 6 membered carbocyclic ring;
R⁷ is selected from D, CD₃, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, (4-10 membered heterocycloalkyl)-C₁₋₆ alkylene, CN, C(O)R^{b7}, C(O)NR^{c7}R^{d7}, C(O)NR^{c7}(ORd^{d7}), C(O)OR^{d7}, C(═NR^{e7})R^{b7}, C(═NR^{e7})NR^{c7}R^{d7}, S(O)R^{b7}, S(O)NR^{c7}R^{d7}, S(O)₂R^{b7}, and S(O)₂NR^{c7}R^{d7}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R⁷ are each optionally substituted with 1, 2, 3, 4, 5, 6, 7, or 8 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R^{a7}, R^{c7}, and R^{d7} are each optionally substituted with 1, 2, 3, 4, 5, 6, 7, or 8 independently selected R^{7A} substituents;
or any R^{c7} and R^{d7} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{7A} substituents;
each R^{b7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R^{b7} are each optionally substituted with 1, 2, 3, 4, 5, 6, 7, or 8 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene;
R^{7A} is selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, (4-7 membered heterocycloalkyl)-C₁₋₆ alkylene, CN, NO₂, OR^{a71}, SR^{a71}, NHOR^{a71}, C(O)R^{b71}, C(O)NR^{e71}R^{d71}, C(O)NR^{e71} (OR^{a71}), C(O)OR^{d71}, OC(O)R^{b71}, OC(O)NR^{e71}R^{d71}, NR^{e71}R^{d71}, NR^{e71}NR^{e71}R^{d71} NR^{c71}C(O)R^{b71}, NR^{c71}C(O)OR^{d71}, NR⁷¹C(O)NR^{c71}R^{d71}, C(═NR^{e71})R^{b71}, C(═NR^{e71})NR^{c71}R^{d71}, NR^{c71}C(═NR^{e71})NR^{c71}R^{d71}, NR^{c71}C(═NR^{e71})R^{b71}, NR^{c71}S(O)R^{b71}, NR^{c71}S(O)NR^{e71}R^{d71}, NR^{c71}S(O)₂R^{b71}, NR^{c71}S(O)(═NR^{e71})R^{b71}, NR^{e71}S(O)₂NR^{e71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, and OS(O)₂R^{b71}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene of R^{7A} are each optionally substituted with 1, 2, 3, or 4 independently selected R^{M} substituents;
each R^{7A} is selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, (4-7 membered heterocycloalkyl)-C₁₋₆ alkylene, CN, NO₂, OR^{a71}, SR^{a71}, NHOR^{a71}, C(O)R^{b71}, C(O)NR^{c71}R^{d71}, C(O)NR^{e71} (OR^{a71}), C(O)OR^{d71}, OC(O)R^{b71}, OC(O)NR^{c71}R^{d71}, NR^{e71}R^{d71}, NR^{e71}NR^{e71}R^{d71} NR^{c71}C(O)R^{b71}, NR^{c71}C(O)OR^{d71}, NR^{c71}C(O)NR^{c71}R^{d71}, C(═NR^{e71})R^{b71}, C(═NR^{e71})NR⁷¹R^{d71}, NR^{c71}C(═NR^{e71})NR^{c71}R^{d71}, NR^{e71}C(═NR^{e71})R^{b71}, NR^{c71}S(O)R^{b71}, NR^{c71}S(O)NR^{c71}R^{d71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71} S(O)₂R^{b71}, S(O)₂NR^{c71}R^{d71}, OS(O)(═NR^{e71})R^{b71}, and OS(O)₂R^{b71}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{7A} are each optionally substituted with 1, 2, 3, or 4 independently selected R^{M} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{a71}, R^{c71}, and R^{d71} are each optionally substituted with 1, 2, 3, or 4 independently selected R^{M} substituents;
or any R^{e7} and R^{d71} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b71} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{b71} are each optionally substituted with 1, 2, 3, or 4 independently selected R^{M} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene; and
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene.

Embodiment 2. The compound of Embodiment 1, or a pharmaceutically acceptable salt or stereoisomer thereof, wherein
Y is CH₂;
R₁ is H, C₁-C₆alkyl or halo;
R₂ is H, halo, C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₄ is H, C₁-C₆alkyl or C₁-C₆alkyl-phenyl;
R₁₀ is —NHC(═O)R₅, —C(═O)NHR, or a 9 or 10 membered bicyclic heteroaryl having 1 to 4 heteroatoms as ring members each independently selected from N, O and S, wherein the bicyclic heteroaryl is unsubstituted or the bicyclic heteroaryl is substituted with one or more R₆;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from C₁-C₆alkyl, —C(═O)NH—C₁-C₆alkyl, and —NHC(═O) C₁-C₆alkyl;
R₁₁ is H; and
R⁷ is D, CD₃, or C₁-C₆alkyl.

Embodiment 3. The compound of Embodiment 1, or a pharmaceutically acceptable salt or stereoisomer thereof, wherein
Y is CH₂;
R₁ is H, C₁-C₆alkyl or halo;
R₂ is H, halo, C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₄ is H, C₁-C₆alkyl or C₁-C₆alkyl-phenyl;
R₁₀ is —NHC(═O)R₅, —C(═O)NHR₅ or a 9 or 10 membered bicyclic heteroaryl having 1 to 4 heteroatoms as ring members each independently selected from N, O and S, wherein the bicyclic heteroaryl is unsubstituted or the bicyclic heteroaryl is substituted with one or more R₆;
R₅ is:

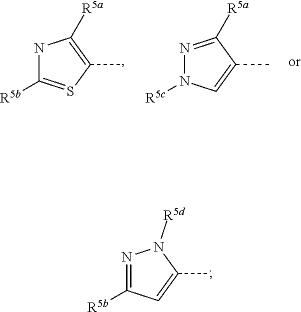

wherein
R^{5a} is C₁-C₆alkyl, C₁-C₆haloalkyl or halo;
R^{5b} is —C(O)—NH—C₁-C₆alkyl, —C(O)NH₂, —C(O)NH-CD₃, —C(O)NH—C₁-C₆haloalkyl, —C(O)NHphenyl, —NHC(═O) C₁-C₆alkyl, C₁-C₆alkoxy, or C₁-C₆haloalkoxy; wherein and each C₁-C₆alkyl is optionally substituted by one or more D;
R^{5c} is 5- or 6-membered ring heteroaryl optionally substituted with halo, C₁-C₆alkyl, C₁-C₆haloakyl, C₁-C₆alkoxy, C₁-C₆haloakoxy or C₃-C₆cycloalkyl;
R^{5d} is C₁-C₆alkyl or C₁-C₆haloalkyl;
R₁₁ is H; and
R⁷ is D, CD₃, or C₁-C₆alkyl.

In an embodiment, R^{5b} is —C(O)—NH—C₁-C₆alkyl, —C(O)NH₂, —C(O)NH-CD₃, —C(O)NH—C₁-C₆haloalkyl, —C(O)NHphenyl, —NHC(═O) C₁-C₆alkyl, C₁-C₆alkoxy, or C₁-C₆haloalkoxy.

Embodiment 3A. The compound of Embodiment 3, or a pharmaceutically acceptable salt or stereoisomer thereof, wherein

### R₅ is:

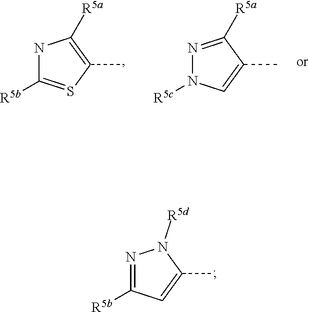

wherein
R^{5a} is C₁-C₆alkyl, C₁-C₆haloalkyl or halo;
R^{5b} is —C(O)—NH—C₁-C₆alkyl, —C(O)NH₂, —C(O)NH-CD₃, —C(O)NH—C₁-C₆haloalkyl, —C(O)NHphenyl, —NHC(═O) C₁-C₆alkyl, C₁-C₆alkoxy, or C₁-C₆haloalkoxy; wherein each C₁-C₆alkyl is optionally substituted by one or more D;
R^{5c} is 5- or 6-membered ring heteroaryl optionally substituted with halo, C₁-C₆alkyl, C₁-C₆haloakyl, C₁-C₆alkoxy, C₁-C₆haloakoxy or C₃-C₆cycloalkyl; and
R^{5d} is C₁-C₆alkyl or C₁-C₆haloalkyl.

In an embodiment, R^{5b} is —C(O)—NH—C₁-C₆alkyl, —C(O)NH₂, —C(O)NH-CD₃, —C(O)NH—C₁-C₆haloalkyl, —C(O)NHphenyl, —NHC(═O) C₁-C₆alkyl, C₁-C₆alkoxy, or C₁-C₆haloalkoxy.

Embodiment 4. The compound of Embodiment 1, or a pharmaceutically acceptable salt or stereoisomer thereof, wherein
Y is CH₂;
R₁ is H, C₁-C₆alkyl or halo;
R₂ is H, halo, C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₄ is H, C₁-C₆alkyl or C₁-C₆alkyl-phenyl;
R₁₀ is —NHC(═O)R₅, —C(═O)NHR₅ or a 9 or 10 membered bicyclic heteroaryl having 1 to 4 heteroatoms as ring members each independently selected from N, O and S, wherein the bicyclic heteroaryl is unsubstituted or the bicyclic heteroaryl is substituted with one or more R₆;
R₅ is:

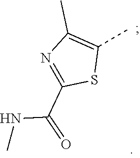

R₁₁ is H; and
R⁷ is D, CD₃, or C₁-C₆alkyl.

Embodiment 5. The compound of Embodiment 1, or a pharmaceutically acceptable salt or stereoisomer thereof, wherein
Y is CH₂;
R₁ is H;
R₂ is H;
R₃ is C₁-C₆alkyl;
R₄ is H;
R₁₀ is —NHC(═O)R₅;
R₅ is 5-membered heteroaryl optionally substituted with one or more substituents independently selected from C₁-C₆alkyl, —C(═O)NH—C₁-C₆alkyl, and —NHC(═O) C₁-C₆alkyl;
R₁ is H; and
R⁷ is D or methyl.

Embodiment 6. The compound of Embodiment 1, or a pharmaceutically acceptable salt or stereoisomer thereof, wherein R₁₀ is —NHC(—O)R₅.

Embodiment 7. The compound of Embodiment 1, or a pharmaceutically acceptable salt or stereoisomer thereof, wherein R₁₀ is —C(═O)NHR₅.

Embodiment 8. The compound of Embodiment 1, or a pharmaceutically acceptable salt or stereoisomer thereof, wherein R₁₀ is a 9 or 10 membered bicyclic heteroaryl having 1 to 4 heteroatoms as ring members each independently selected from N, O and S, wherein the bicyclic heteroaryl is unsubstituted or the bicyclic heteroaryl is substituted with one or more R₆.

Embodiment 9. The compound of Embodiment 1, or a pharmaceutically acceptable salt or stereoisomer thereof, wherein R₁₀ is

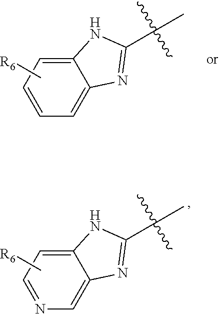

wherein R₆ is H, C₁-C₆alkyl, C₁-C₆alkoxy, C₁-C₆haloalkyl, cyano or halo.

Embodiment 10. The compound of any one of Embodiments 1 to 5, or a pharmaceutically acceptable salt or stereoisomer thereof, wherein R₄ is H.

Embodiment 11. The compound of any one of Embodiments 1 to 5, or a pharmaceutically acceptable salt or stereoisomer thereof, wherein R₄ is C₁-C₆alkyl.

Embodiment 12. The compound of any one of Embodiments 1 to 5, or a pharmaceutically acceptable salt or stereoisomer thereof, wherein R₄ is C₁-C₆alkyl-phenyl.

Embodiment 13. The compound of Embodiment 1, or a pharmaceutically acceptable salt or stereoisomer thereof, having a structure of Formula (II)

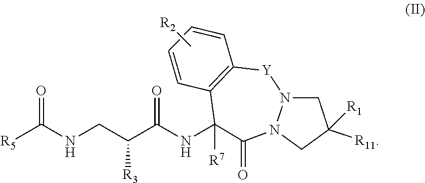

Embodiment 14. The compound of Embodiment 13, or a pharmaceutically acceptable salt or stereoisomer thereof, wherein
Y is CH₂ or C═O;
R₁ is H, C₁-C₆alkyl or halo;
R₂ is H, halo, C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by one or more D or C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo;
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy; and
xxi) —C(═O)NH₂;
R₁₁ is H, C₁-C₆alkyl or halo; or
R₁ and R₁₁ together with the carbon atom to which they are attached, may form a 3 to 6 membered carbocyclic ring;
R⁷ is selected from D, CD₃, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, (4-10 membered heterocycloalkyl)-C₁₋₆ alkylene, CN, C(O)R^{b7}, C(O)NR^{c7}R^{d7}, C(O)NR^{c7}(ORd^{d7}), C(O)OR^{d7}, C(═NR^{e7})R^{b7}, C(═NR^{e7})NR^{c7}R^{d7}, S(O)R^{b7}, S(O)NR^{c7}R^{d7}, S(O)₂R^{b7}, and S(O)₂NR^{c7}R^{d7}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R⁷ are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene;
each R^{b7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene;
R^{7A} is selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene; and each R^{7A} is selected from halo, CN, NO₂, OH, C₁₋₆ alkyl, C₁₋₆ haloalkyl, C₁₋₆ alkoxy, and C₁-6 haloalkoxy.

Embodiment 14A. The compound of Embodiment 13, or a pharmaceutically acceptable salt or stereoisomer thereof, wherein
Y is CH₂ or C═O;
R₁ is H, C₁-C₆alkyl or halo;
R₂ is H, halo, C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloakoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo; and
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy;
each R₆ is independently selected from C₁-C₆alkyl, C₁-C₆alkoxy, C₁-C₆haloalkyl, cyano and halo;
R₁₁ is H, C₁-C₆alkyl or halo; or
R₁ and R₁₁ together with the carbon atom to which they are attached, may form a 3 to 6 membered carbocyclic ring;
R⁷ is selected from D, CD₃, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, (4-10 membered heterocycloalkyl)-C₁₋₆ alkylene, CN, C(O)R^{b7}, C(O)NR^{c7}R^{d7}, C(O)NR^{c7}(OR^{a7}), C(O)OR^{d7}, C(═NR^{e7})R^{b7}, C(═NR^{e7})NR^{c7}R^{d7}, S(O)R^{b7}, S(O)NR^{c7}R^{d7}, S(O)₂R^{b7}, and S(O)₂NR^{c7}R^{d7}; wherein the C₁₋₆ alkyl, C₂₋₆ alkenyl, C₂₋₆ alkynyl, C₆₋₁₀ aryl, C₃₋₁₀ Cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R⁷ are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene;
each R^{b7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, and (4-10 membered heterocycloalkyl)-C₁₋₆ alkylene;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene;
R^{7A} is selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene; and
each R^{7A} is selected from halo, CN, NO₂, OH, C₁₋₆ alkyl, C₁₋₆ haloalkyl, C₁₋₆ alkoxy, and C₁₋₆ haloalkoxy.

Embodiment 15. The compound of Embodiment 1, or a pharmaceutically acceptable salt or stereoisomer thereof, having a structure of Formula (III)

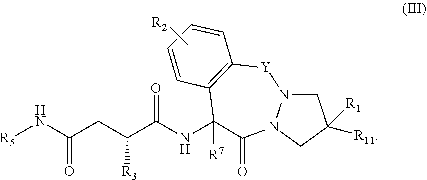

Embodiment 16. The compound of Embodiment 15, or a pharmaceutically acceptable salt or stereoisomer thereof,

wherein
Y is CH₂ or C═O;
R₁ is H, C₁-C₆alkyl or halo;
R₂ is H, halo, C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloalkoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₅alkoxy or a 4 to 6-member heterocyclyl optionally substituted with oxo; and
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy;
each R₆ is independently selected from C₁-C₆alkyl, C₁-C₆alkoxy, C₁-C₆haloalkyl, cyano and halo;
R₁₁ is H, C₁-C₆alkyl or halo; or
R₁ and R₁₁ together with the carbon atom to which they are attached, may form a 3 to 6 membered carbocyclic ring;
R⁷ is selected from D, CD₃, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, (4-10 membered heterocycloalkyl)-C₁₋₆ alkylene, CN, C(O)R^{b7}, C(O)NR^{c7}R^{d7}, C(O)NR^{c7}(ORd^{d7}), C(O)OR^{d7}, C(═NR^{e7})R^{b7}, C(═NR^{e7})NR^{c7}R^{d7}, S(O)R^{b7}, S(O)NR^{c7}R^{d7}, S(O)₂R^{b7}, and S(O)₂NR^{c7}R^{d7}; wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R⁷ are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl;
each R^{b7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl; and
each R^{7A} is selected from halo, CN, NO₂, OH, C₁₋₆ alkyl, C₁₋₆ haloalkyl, C₁₋₆ alkoxy, and C₁₋₆ haloalkoxy.

Embodiment 17. The compound of Embodiment 1, or a pharmaceutically acceptable salt or stereoisomer thereof, having a structure of Formula (IV):

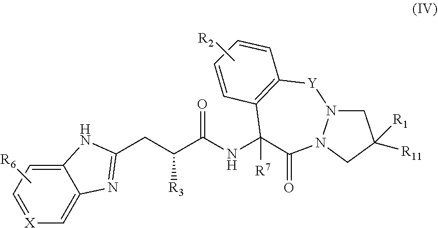

wherein
X is CH or N;
R₁ is H, C₁-C₆alkyl or halo;
R₂ is H, halo, C₁-C₆alkyl, C₁-C₆haloalkyl, C₁-C₆alkyloxy, and C₁-C₆haloalkyloxy;
Y is CH₂ or C(O);
R₃ is H, C₁-C₆haloalkyl, C₃-C₆cycloalkyl, C₁-C₆alkyl-phenyl or C₁-C₆alkyl optionally substituted with C₁-C₆alkoxy;
X is CH or N;
R₆ is H, C₁-C₆alkyl, C₁-C₆alkoxy, C₁-C₆haloalkyl, cyano or halo;
R₁₁ is H, C₁-C₆alkyl or halo; or
R₁ and R₁₁ together with the carbon atom to which they are attached may form a 3 to 6 membered carbocyclic ring;
R⁷ is selected from D, CD₃, halo, C₁₋₆ alkyl, C₁₋₆ haloalkyl, C₂₋₆ alkenyl, C₂₋₆ alkynyl, C₆₋₁₀ aryl-C₁₋₆ alkylene, (5-10 membered heteroaryl)-C₁₋₆ alkylene, CN, C(O)R^{b7}, C(O)NR^{c7}R^{d7}, C(O)NR^{c7}(ORd^{d7}), C(O)OR^{d7}, C(═NR^{e7})R^{b7}, C(═NR^{e7})NR^{c7}R^{d7}, S(O)R^{b7}, S(O)NR^{c7}R^{d7}, S(O)₂R^{b7}, and S(O)₂NR^{c7}R^{d7};, wherein the C₆₋₁₀ aryl-C₁₋₆ alkylene and (5-10 membered heteroaryl)-C₁₋₆ alkylene of R⁷ are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl;
each R^{b7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl; and each R^{7A} is selected from halo, CN, NO₂, OH, C₁₋₆ alkyl, C₁₋₆ haloalkyl, C₁₋₆ alkoxy, and C₁₋₆ haloalkoxy.

Embodiment 18. The compound of any one of Embodiments 1 to 17, or a pharmaceutically acceptable salt or stereoisomer thereof, wherein R₁ is H.

Embodiment 19. The compound of any one of Embodiments 1 to 17, or a pharmaceutically acceptable salt or stereoisomer thereof, wherein R₁ is C₁-C₆alkyl.

Embodiment 20. The compound of any one of Embodiments 1 to 17, or a pharmaceutically acceptable salt or stereoisomer thereof, wherein R₁ is halo.

Embodiment 21. The compound of any one of Embodiments 1 to 20, or a pharmaceutically acceptable salt or stereoisomer thereof, wherein Ru is H.

Embodiment 22. The compound of any one of Embodiments 1 to 21, or a pharmaceutically acceptable salt or stereoisomer thereof, wherein Ru is C₁-C₆alkyl.

Embodiment 23. The compound of any one of Embodiments 1 to 21, or a pharmaceutically acceptable salt or stereoisomer thereof, wherein Ru is halo.

Embodiment 24. The compound of any one of Embodiments 1 to 17, or a pharmaceutically acceptable salt or stereoisomer thereof, wherein R₁ and Ru together with the carbon atom to which they are attached, form a 3 to 6 membered carbocyclic ring.

Embodiment 25. The compound of any one of Embodiments 1 to 17, or a pharmaceutically acceptable salt or stereoisomer thereof, wherein R₁ and Rn together with the carbon atom to which they are attached, form a cyclopropyl ring.

Embodiment 26. The compound of any one of Embodiments 1 to 25, or a pharmaceutically acceptable salt or stereoisomer thereof, wherein R₂ is H.

Embodiment 27. The compound of any one of Embodiments 1 to 25, or a pharmaceutically acceptable salt or stereoisomer thereof, wherein R₂ is halo.

Embodiment 28. The compound of any one of Embodiments 1 to 25, or a pharmaceutically acceptable salt or stereoisomer thereof, wherein R₂ is F.

Embodiment 29. The compound of any one of Embodiments 1 to 16, or a pharmaceutically acceptable salt or stereoisomer thereof, having a structure of Formula (IIA):

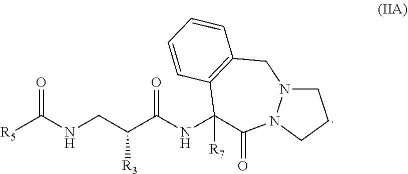

Embodiment 30. The compound of any one of Embodiments 1 to 16, or a pharmaceutically acceptable salt or stereoisomer thereof, having a structure of Formula (IIB):

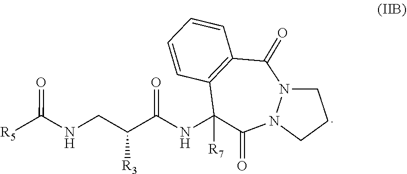

Embodiment 31. The compound of any one of Embodiments 1 to 16, or a pharmaceutically acceptable salt or stereoisomer thereof, having a structure of Formula (IIC):

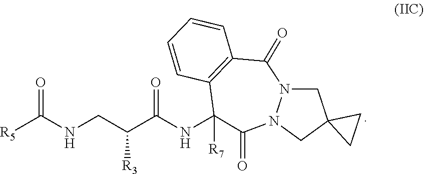

Embodiment 32. The compound of any one of Embodiments 1 to 16, or a pharmaceutically acceptable salt or stereoisomer thereof, having a structure of Formula (IID):

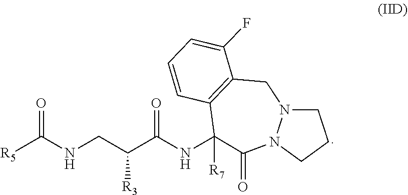

Embodiment 33. The compound of any one of Embodiments 1 to 16, or a pharmaceutically acceptable salt or stereoisomer thereof, having a structure of Formula (IIA), Formula (IIB), Formula (IIC) or Formula (IID):

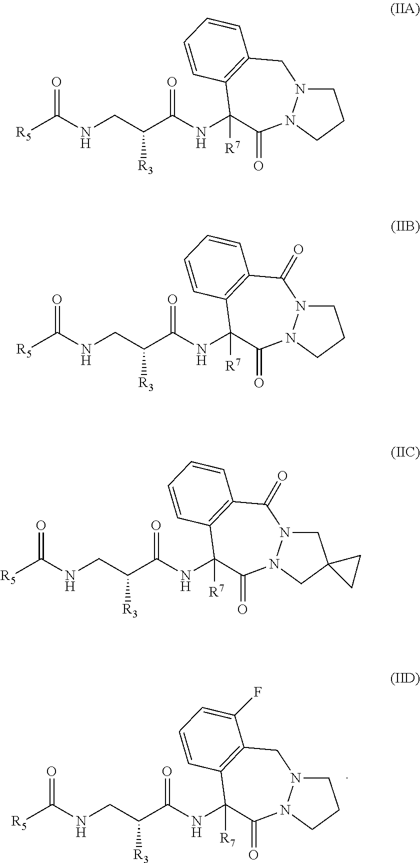

Embodiment 34. The compound of Embodiment 33, or a pharmaceutically acceptable salt or stereoisomer thereof, wherein

R₃ is H, C₁-C₆haloalkyl, C₃-C₆cycloalkyl, C₁-C₆alkyl-phenyl or C₁-C₆alkyl optionally substituted with C₁-C₆alkoxy;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-Cecycloalkyl, C₃-C₆cycloalkenyl and a C₁-C₆alkyl optionally substituted by—OH, C₁-C₆alkoxy or a 4 to 6-member heterocyclyl optionally substituted with oxo; and
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy;
R⁷ is selected from D, CD₃, halo, C₁₋₆ alkyl, C₁₋₆ haloalkyl, C₂₋₆ alkenyl, C₂₋₆ alkynyl, C₆₋₁₀ aryl-C₁₋₆ alkylene, (5-10 membered heteroaryl)-C₁₋₆ alkylene, CN, C(O)R^{b7}, C(O)NR^{c7}R^{d7}, C(O)NR^{c7}(ORd^{d7}), C(O)OR^{d7}, C(═NR^{e7})R^{b7}, C(═NR^{e7})NR^{c7}R^{d7}, S(O)R^{b7}, S(O)NR^{c7}R^{d7}, S(O)₂R^{b7}, and S(O)₂NR^{c7}R^{d7}, wherein the C₆₋₁₀ aryl-C₁₋₆ alkylene and (5-10 membered heteroaryl)-C₁₋₆ alkylene of R⁷ are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl;
each R^{b7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl; and each R^{7A} is selected from halo, CN, NO₂, OH, C₁₋₆ alkyl, C₁₋₆ haloalkyl, C₁₋₆ alkoxy, and C₁₋₆ haloalkoxy.

Embodiment 35. The compound of any one of Embodiments 1 to 16, or a pharmaceutically acceptable salt or stereoisomer thereof, having a structure of Formula (IIIA):

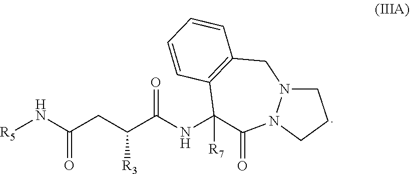

Embodiment 36. The compound of any one of Embodiments 1 to 16, or a pharmaceutically acceptable salt or stereoisomer thereof, having a structure of Formula (IIIB):

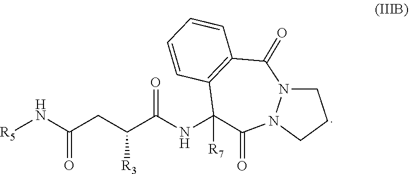

Embodiment 37. The compound of any one of Embodiments 1 to 16, or a pharmaceutically acceptable salt or stereoisomer thereof, having a structure of Formula (IIIC):

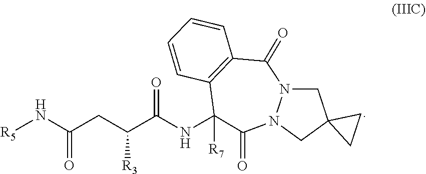

Embodiment 38. The compound of any one of Embodiments 1 to 16, or a pharmaceutically acceptable salt or stereoisomer thereof, having a structure of Formula (IIID):

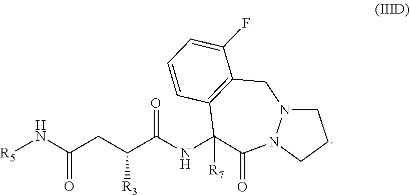

Embodiment 39. The compound of any one of Embodiments 1 to 16, or a pharmaceutically acceptable salt or stereoisomer thereof, having a structure of Formula (IIIA), Formula (IIIB), Formula (IIIC) or Formula (IIID):

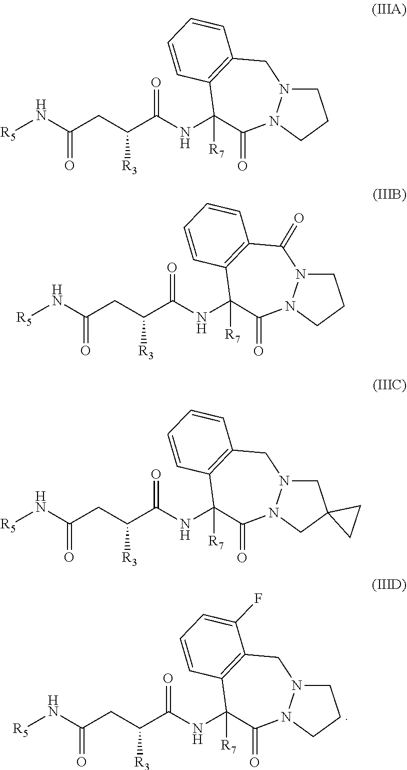

Embodiment 40. The compound of Embodiment 39, wherein
R₃ is H, C₁-C₆haloalkyl, C₃-C₆cycloalkyl, C₁-C₆alkyl-phenyl or C₁-C₆alkyl optionally substituted with C₁-C₆alkoxy;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by one or more D or C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloalkoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo;
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy; and
xxi) —C(═O)NH₂;
R⁷ is selected from D, CD₃, halo, C₁₋₆ alkyl, C₁₋₆ haloalkyl, C₂₋₆ alkenyl, C₂₋₆ alkynyl, C₆₋₁₀ aryl-C₁₋₆ alkylene, (5-10 membered heteroaryl)-C₁₋₆ alkylene, CN, C(O)R^{b7}, C(O)NR^{c7}R^{d7}, C(O)NR⁷ (OR^{a7}), C(O)OR^{d7}, C(═NR^{e7})R^{b7}, C(═NR^{e7})NR^{c7}R^{d7}, S(O)R^{b7}, S(O)NR^{c7}R^{d7}, S(O)₂R^{b7}, and S(O)₂NR^{c7}R^{d7}, wherein the C₆₋₁₀ aryl-C₁₋₆ alkylene and (5-10 membered heteroaryl)-C₁₋₆ alkylene of R⁷ are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl;
each R^{b7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl; and each R^{7A} is selected from halo, CN, NO₂, OH, C₁₋₆ alkyl, C₁₋₆ haloalkyl, C₁₋₆ alkoxy, and C₁₋₆ haloalkoxy.

Embodiment 40A. The compound of Embodiment 39, wherein
R₃ is H, C₁-C₆haloalkyl, C₃-C₆cycloalkyl, C₁-C₆alkyl-phenyl or C₁-C₆alkyl optionally substituted with C₁-C₆alkoxy;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloalkoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo; and
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy;
R⁷ is selected from D, CD₃, halo, C₁₋₆ alkyl, C₁₋₆ haloalkyl, C₂₋₆ alkenyl, C₂₋₆ alkynyl, C₆₋₁₀ aryl-C₁₋₆ alkylene, (5-10 membered heteroaryl)-C₁₋₆ alkylene, CN, C(O)R^{b7}, C(O)NR^{c7}R^{d7}, C(O)NR^{c7}(ORd^{d7}), C(O)OR^{a7}, C(═NR^{e7})R^{b7}, C(═NR^{e7})NR^{c7}R^{d7}, S(O)R^{b7}, S(O)NR^{c7}R^{d7}, S(O)₂R^{b7}, and S(O)₂NR^{c7}R^{d7}, wherein the C₆₋₁₀ aryl-C₁₋₆ alkylene and (5-10 membered heteroaryl)-C₁₋₆ alkylene of R⁷ are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl;
each R^{b7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, and 4-10 membered heterocycloalkyl; and each R^{7A} is selected from halo, CN, NO₂, OH, C₁₋₆ alkyl, C₁₋₆ haloalkyl, C₁₋₆ alkoxy, and C₁₋₆ haloalkoxy.

Embodiment 41. The compound of any one of Embodiments 1 to 40, or a pharmaceutically acceptable salt or stereoisomer thereof, wherein R₃ is H.

Embodiment 42. The compound of any one of Embodiments 1 to 40, or a pharmaceutically acceptable salt or stereoisomer thereof, wherein R₃ is C₁-C₆alkyl.

Embodiment 43. The compound of any one of Embodiments 1 to 40, or a pharmaceutically acceptable salt or stereoisomer thereof, wherein R₃ is methyl, ethyl, propyl or iso-propyl.

Embodiment 44. The compound of any one of Embodiments 1 to 40, or a pharmaceutically acceptable salt or stereoisomer thereof, wherein R₃ is C₁-C₆alolkyl.

Embodiment 45. The compound of any one of Embodiments 1 to 40, or a pharmaceutically acceptable salt or stereoisomer thereof, wherein R₃ is CF₃.

Embodiment 46. The compound of any one of Embodiments 1 to 40, or a pharmaceutically acceptable salt or stereoisomer thereof, wherein R₃ is C₁-C₆alkyl-phenyl.

Embodiment 47. The compound of any one of Embodiments 1 to 40, or a pharmaceutically acceptable salt or stereoisomer thereof, wherein R₃ is —CH₂-phenyl.

Embodiment 48. The compound of any one of Embodiments 1 to 40, or a pharmaceutically acceptable salt or stereoisomer thereof, wherein R₃ is C₃-C₆cycloalkyl.

Embodiment 49. The compound of any one of Embodiments 1 to 40, or a pharmaceutically acceptable salt or stereoisomer thereof, wherein R₃ is cyclopropyl or cyclobutyl.

Embodiment 50. The compound of any one of Embodiments 1 to 40, or a pharmaceutically acceptable salt or stereoisomer thereof, wherein R₃ is C₁-C₆alkyl substituted with C₁-C₆alkoxy. Embodiment 51. The compound of any one of Embodiments 1 to 40, or a pharmaceutically acceptable salt or stereoisomer thereof, wherein R₃ is —CH₂CH₂OCH₃.

Embodiment 52. The compound of any one of Embodiments 1 to 51, or a pharmaceutically acceptable salt or stereoisomer thereof, having a structure of Formula (V):

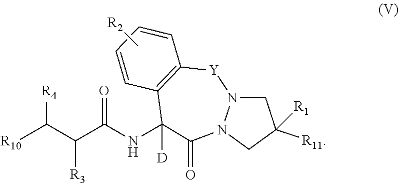

Embodiment 53. The compound of Embodiment 52, or a pharmaceutically acceptable salt or stereoisomer thereof, having a structure of Formula (VA):

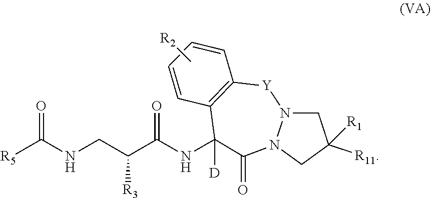

Embodiment 54. The compound of Embodiment 52, or a pharmaceutically acceptable salt or stereoisomer thereof, having a structure of Formula (VB):

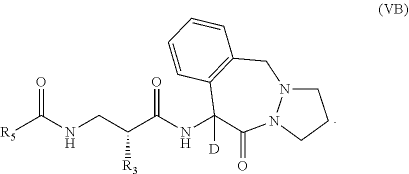

Embodiment 55. The compound of any one of Embodiments 1 to 54, or a pharmaceutically acceptable salt or stereoisomer thereof, wherein R₅ is a 5-membered heteroaryl having 1, 2 or 3 heteroatoms as ring members each independently selected from N, O and S, wherein the 5-membered heteroaryl is unsubstituted.

Embodiment 56. The compound of any one of Embodiments 1 to 54, or a pharmaceutically acceptable salt or stereoisomer thereof, wherein R₅ is a 5-membered heteroaryl having 1, 2 or 3 heteroatoms as ring members each independently selected from N, O and S, wherein the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by one or more D or C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloalkoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo;
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy; and
xxi) —C(═O)NH₂.

Embodiment 56A. The compound of any one of Embodiments 1 to 53, or a pharmaceutically acceptable salt or stereoisomer thereof, wherein R₅ is a 5-membered heteroaryl having 1, 2 or 3 heteroatoms as ring members each independently selected from N, O and S, wherein the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C-C₅alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloalkoxy and C₁-C₆haloalkyl;
xviii) a 4 to 6-member heterocyclyl optionally substituted with oxo, —C(═O)OC1-C₆alkyl or —C(═O)OC1-C₆cycloalkyl; and
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo; and
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy.

Embodiment 57. The compounds of any one of Embodiments 1 to 56, or a pharmaceutically acceptable salt thereof, wherein R₅ is:

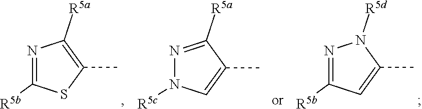

wherein
R^{5a} is C₁-C₆alkyl, C₁-C₆haloalkyl or halo;
R^{5b} is —C(O)—NH—C₁-C₆alkyl, —C(O)NH₂, —C(O)NH-CD₃, —C(O)NH—C₁-C₆haloalkyl, —C(O)NHphenyl, —NHC(═O) C₁-C₆alkyl, C₁-C₆alkoxy, C₁-C₆haloalkoxy, C₃-C₆cycloalkyl, C₃-C₆cycloalkenyl, 4- to 6-membered heterocyclyl, 5- or 6-membered ring heteroaryl; wherein heteroaryl is optionally substituted with halo, C₁-C₆alkyl, C₁-C₆haloakyl, C₁-C₆alkoxy, C₁-C₆haloakoxy or C₃-C₆cycloalkyl; wherein heterocyclyl is optionally substituted with oxo, —C(O)O—C₁-C₆alkyl or —C(O)O—C₃-C₆cycloalkyl; —C(O)NHphenyl is optionally substituted with halo or C₁-C₆alkyl; and each C₁-C₆alkyl is optionally substituted by one or more D;
R^{5c} is 5- or 6-membered ring heteroaryl optionally substituted with halo, C₁-C₆alkyl, C₁-C₆haloakyl, C₁-C₆alkoxy, C₁-C₆haloakoxy or C₃-C₆cycloalkyl; and
R^{5d} is C₁-C₆alkyl or C₁-C₆haloalkyl.

In an embodiment, R^{5b} is —C(O)—NH—C₁-C₆alkyl, —C(O)NH₂, —C(O)NH-CD₃, —C(O)NH—C₁-C₆haloalkyl, —C(O)NHphenyl, —NHC(═O) C₁-C₆alkyl, C₁-C₆alkoxy, C₁-C₆haloalkoxy, C₃-Cecycloalkyl, C₃-C₆cycloalkenyl, 4- to 6-membered heterocyclyl, 5- or 6-membered ring heteroaryl; wherein heteroaryl is optionally substituted with halo, C₁-C₆alkyl, C₁-C₆haloakyl, C₁-C₆alkoxy, C₁-C₆haloakoxy or C₃-C₆cycloalkyl; and wherein heterocyclyl is optionally substituted with oxo, —C(O)O—C₁-C₆alkyl or —C(O)O—C₃-C₆cycloalkyl; and wherein-C(O)NHphenyl is optionally substituted with halo or C₁-C₆alkyl.

Embodiment 57A. The compounds of any one of Embodiments 1 to 56, or a pharmaceutically acceptable salt thereof, wherein R₅ is:

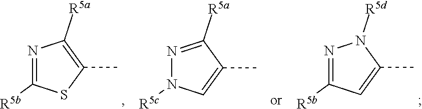

wherein
R^{5a} is C₁-C₆alkyl, C₁-C₆haloalkyl or halo;
R^{5b} is —C(O)—NH—C₁-C₆alkyl, —C(O)NH—C₁-C₆haloalkyl, —C(O)NHphenyl, —NHC(═O) C₁-C₆alkyl, C₁-C₆alkoxy, C₁-C₆haloalkoxy, C₃-C₆cycloalkyl, C₃-C₆cycloalkenyl, 4- to 6-membered heterocyclyl, 5- or 6-membered ring heteroaryl; wherein heteroaryl is optionally substituted with halo, C₁-C₆alkyl, C₁-C₆haloakyl, C₁-C₆alkoxy, C₁-C₆haloakoxy or C₃-C₆cycloalkyl; heterocyclyl is optionally substituted with oxo, —C(O)O—C₁-C₆alkyl or —C(O)O—C₃-C₆cycloalkyl; —C(O)NHphenyl is optionally substituted with halo or C₁-C₆alkyl; and each C₁-C₆alkyl is optionally substituted by one or more D.
R^{5c} is 5- or 6-membered ring heteroaryl optionally substituted with halo, C₁-C₆alkyl, C₁-C₆haloakyl, C₁-C₆alkoxy, C₁-C₆haloakoxy or C₃-C₆cycloalkyl; and
R^{5d} is C₁-C₆alkyl or C₁-C₆haloalkyl.

In an embodiment, R^{5b} is —C(O)—NH—C₁-C₆alkyl, —C(O)NH—C₁-C₆haloalkyl, —C(O)NHphenyl, —NHC(═O) C₁-C₆alkyl, C₁-C₆alkoxy, C₁-C₆haloalkoxy, C₃-C₆cycloalkyl, C₃-C₆cycloalkenyl, 4- to 6-membered heterocyclyl, 5- or 6-membered ring heteroaryl; wherein heteroaryl is optionally substituted with halo, C₁-C₆alkyl, C₁-C₆haloakyl, C₁-C₆alkoxy, C₁-C₆haloakoxy or C₃-C₆cycloalkyl; and wherein heterocyclyl is optionally substituted with oxo, —C(O)O—C₁-C₆alkyl or —C(O)O—C₃-C₆cycloalkyl; and wherein-C(O)NHphenyl is optionally substituted with halo or C₁-C₆alkyl.

Embodiment 58. The compound of any one of Embodiments 1 to 56, or a pharmaceutically acceptable salt or stereoisomer thereof, wherein R₅ is

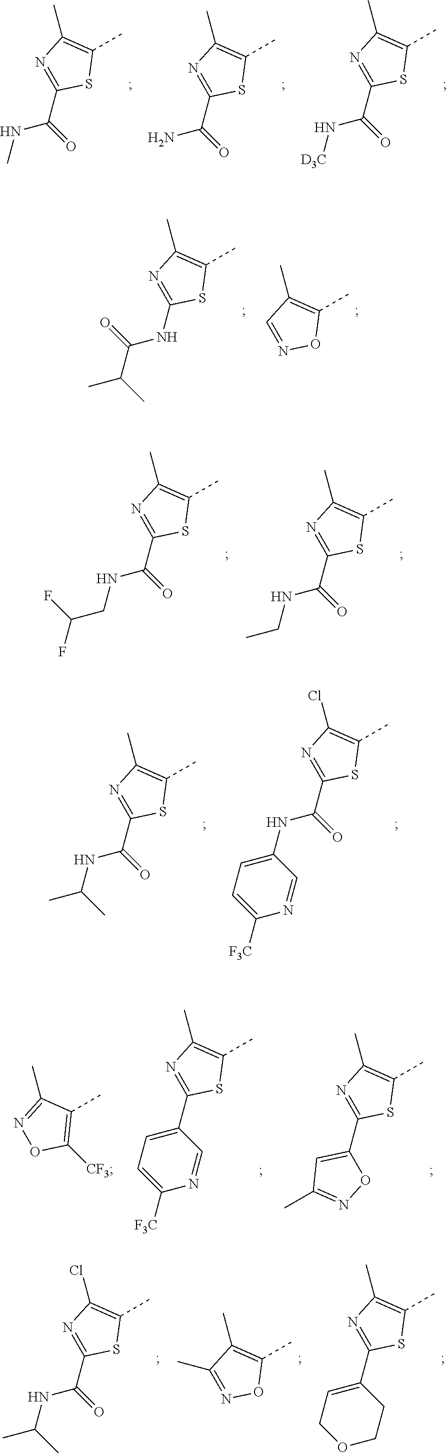

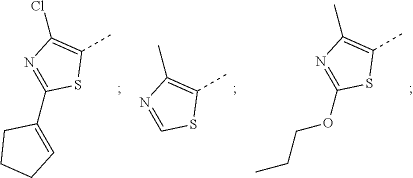

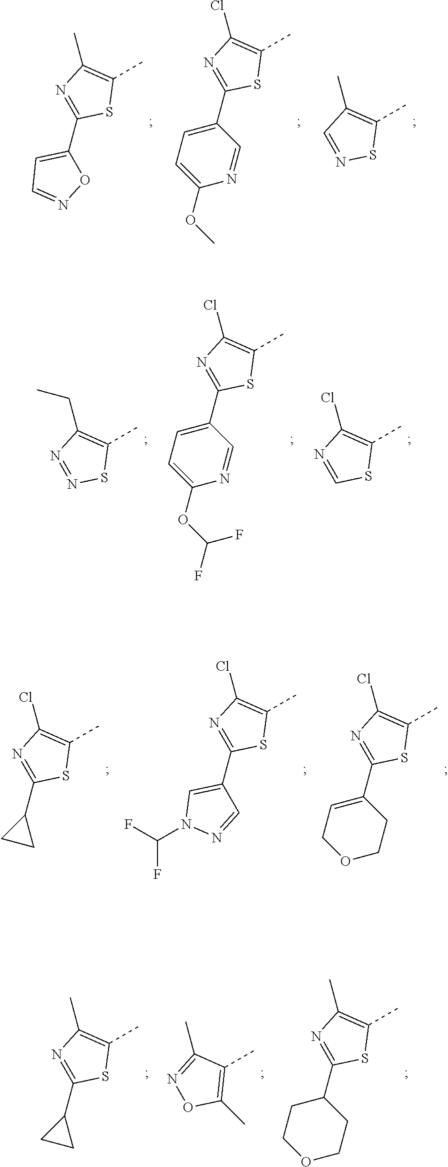

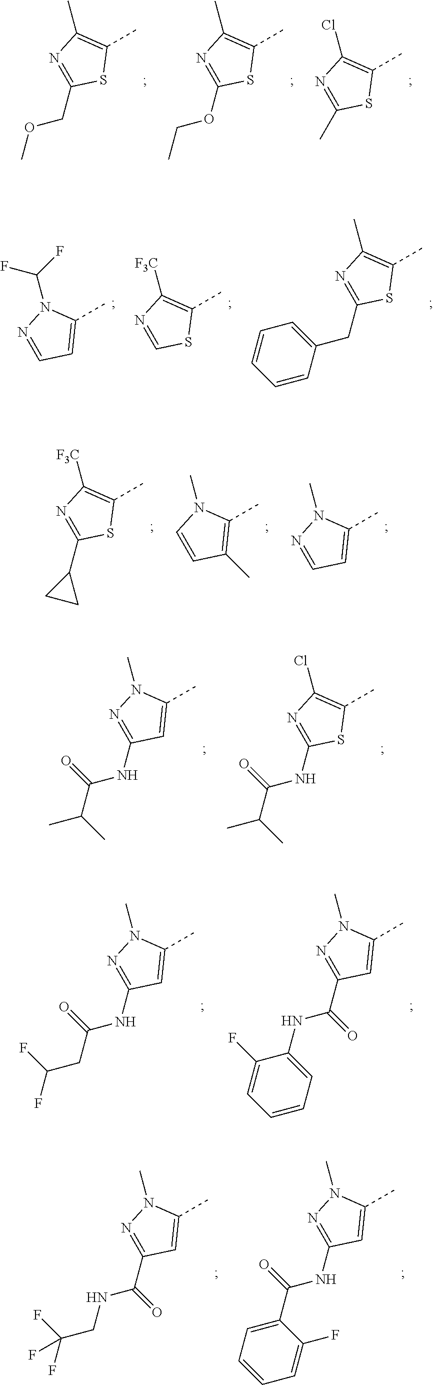

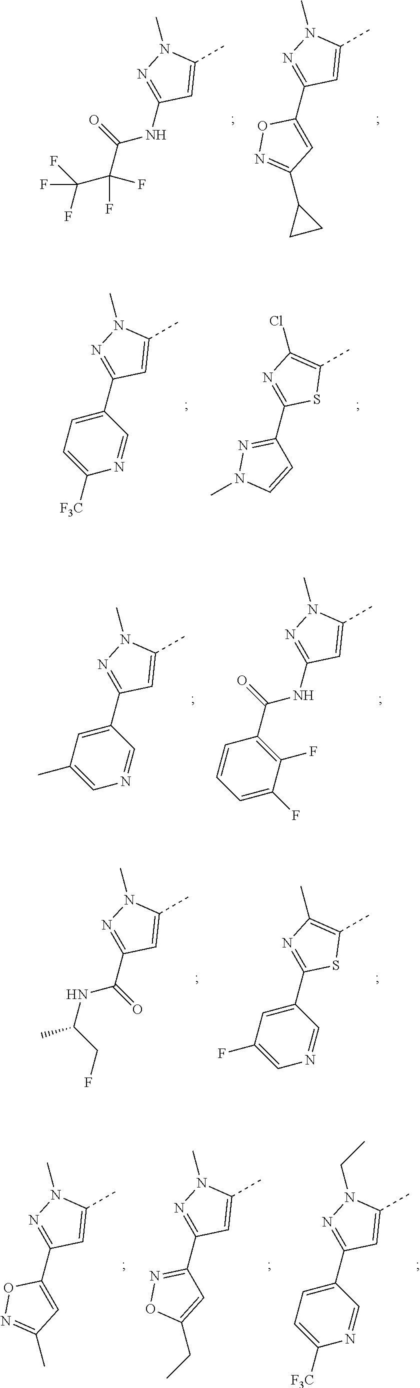

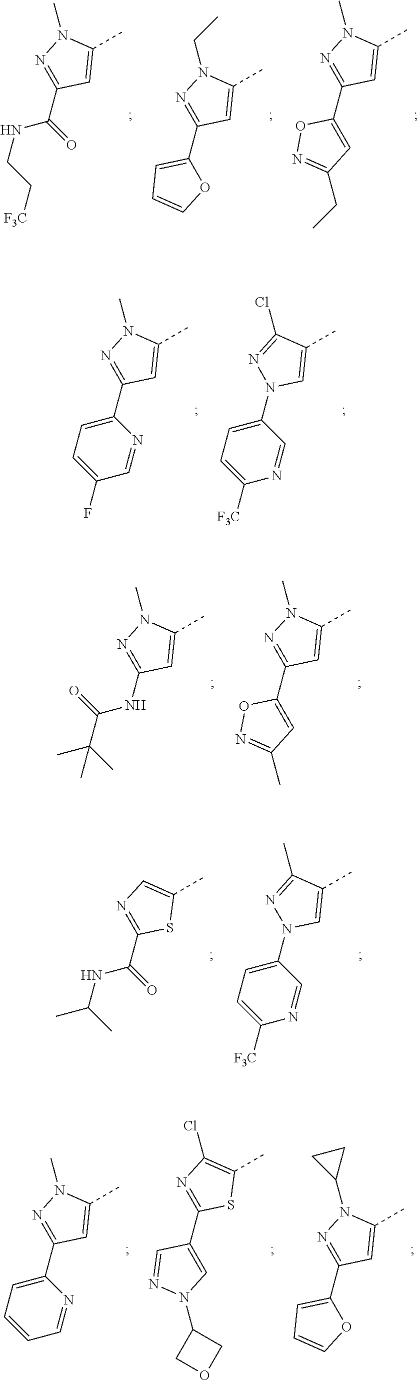

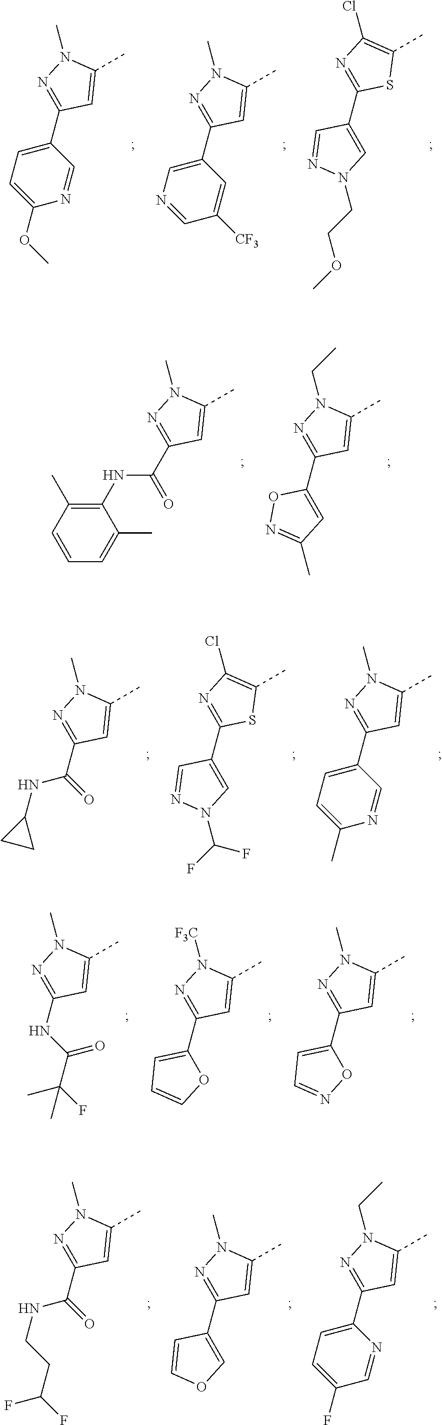

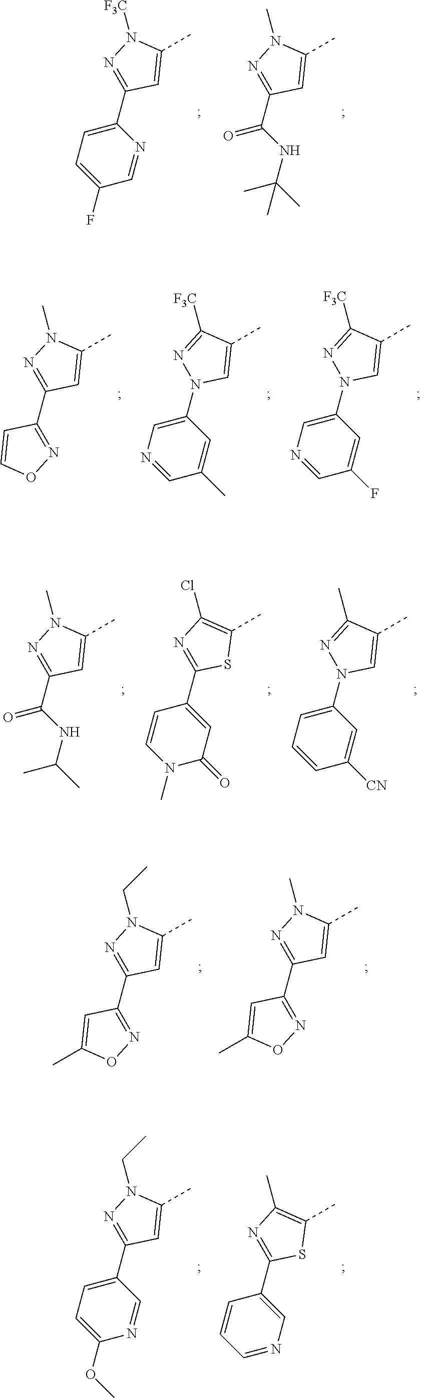

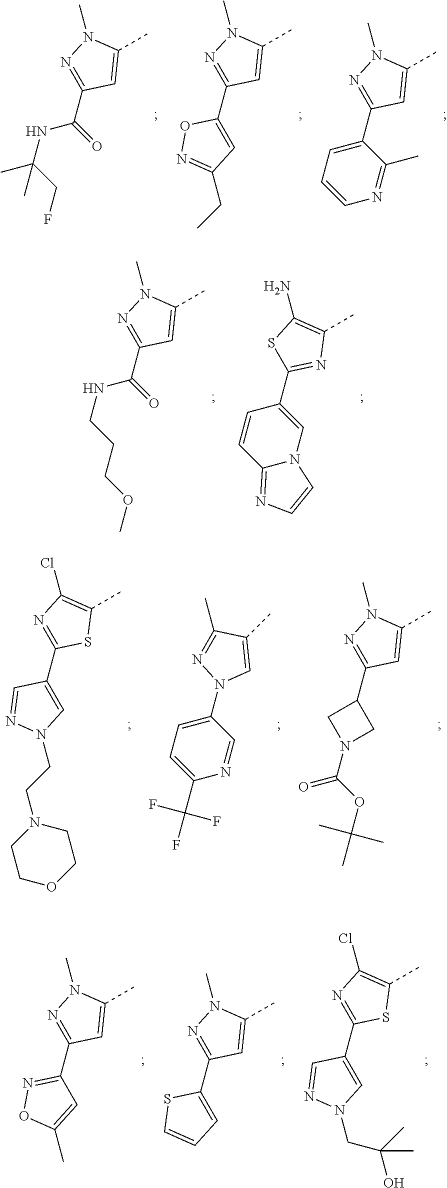

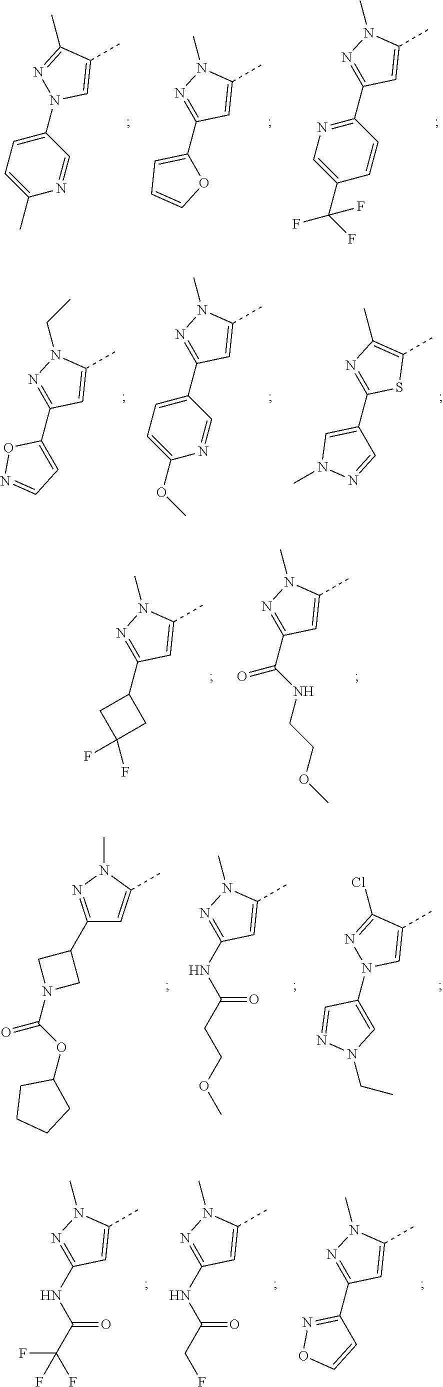

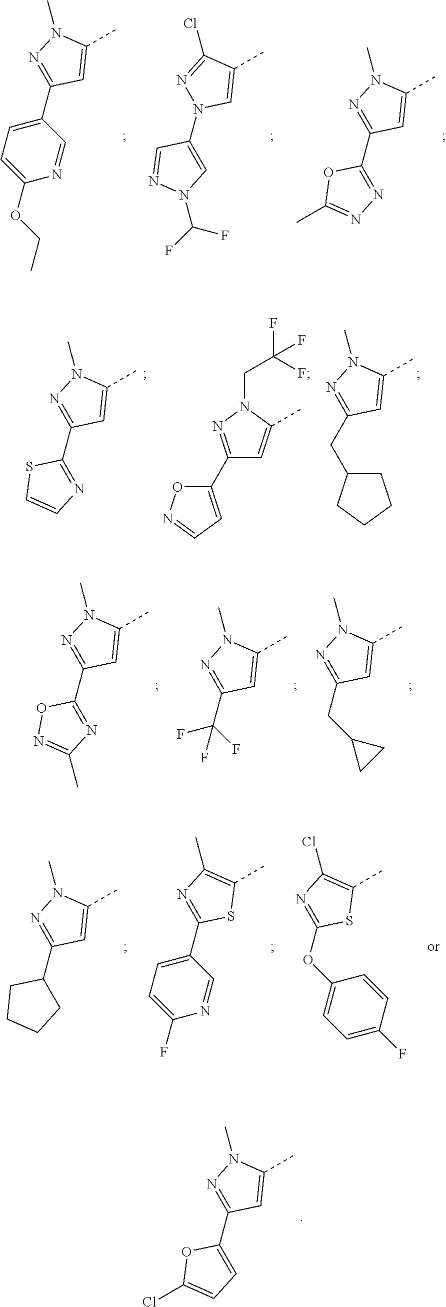

Embodiment 58A. The compound of any one of Embodiments 1 to 56, or a pharmaceutically acceptable salt or stereoisomer thereof, wherein R₅ is

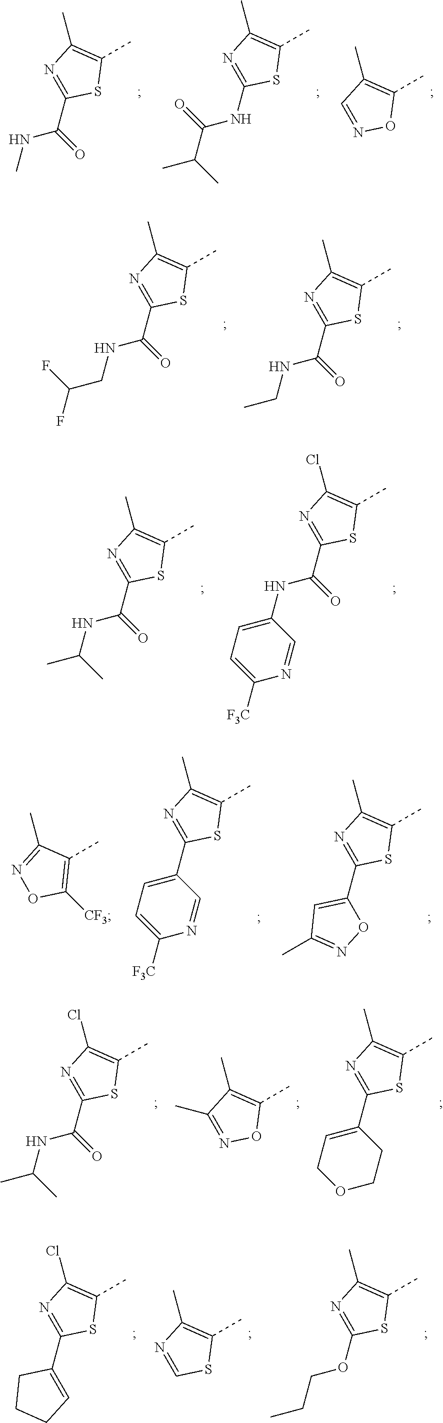

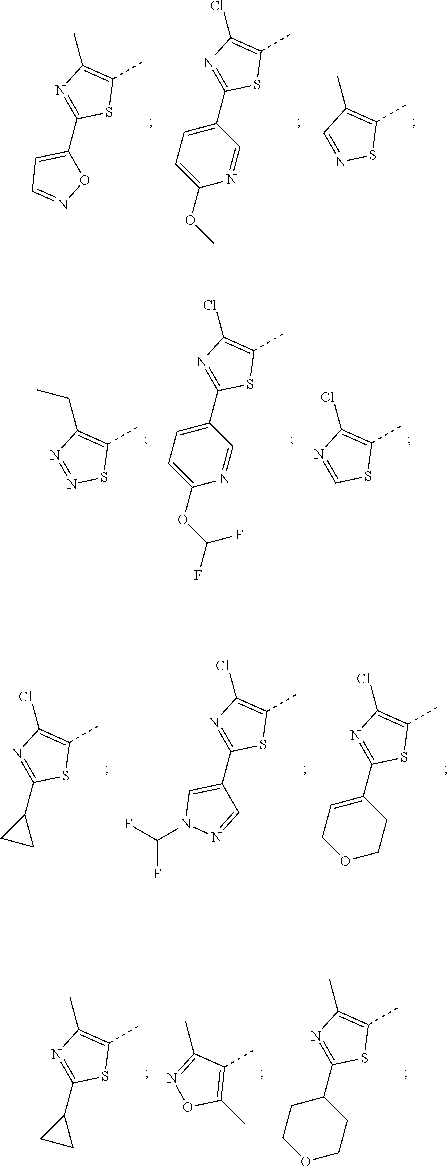

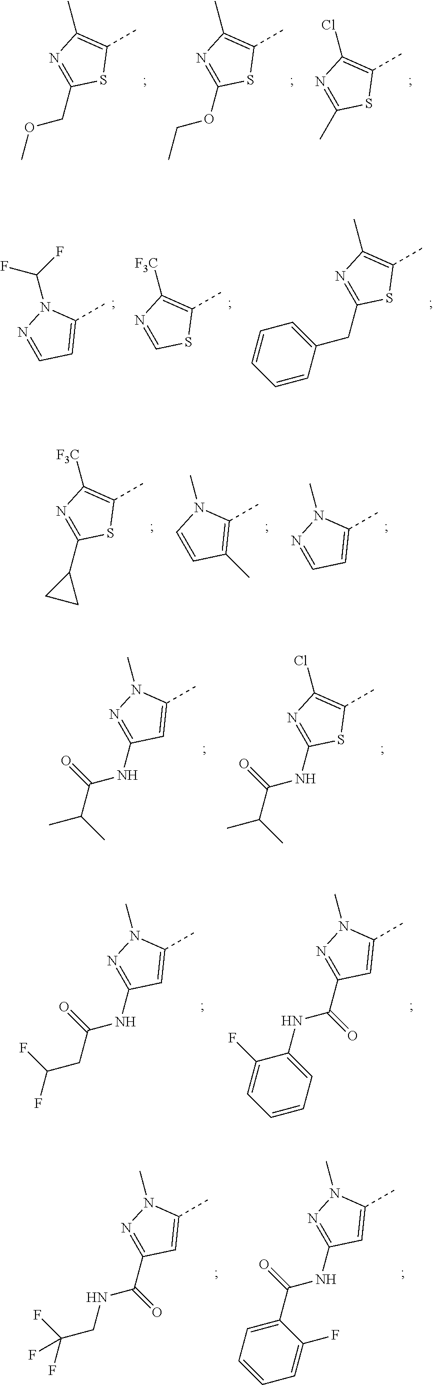

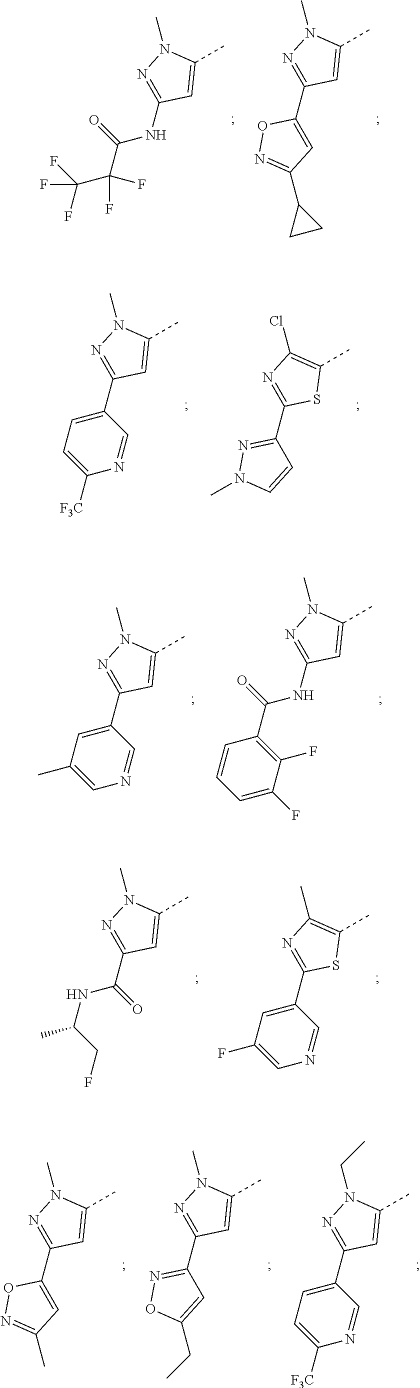

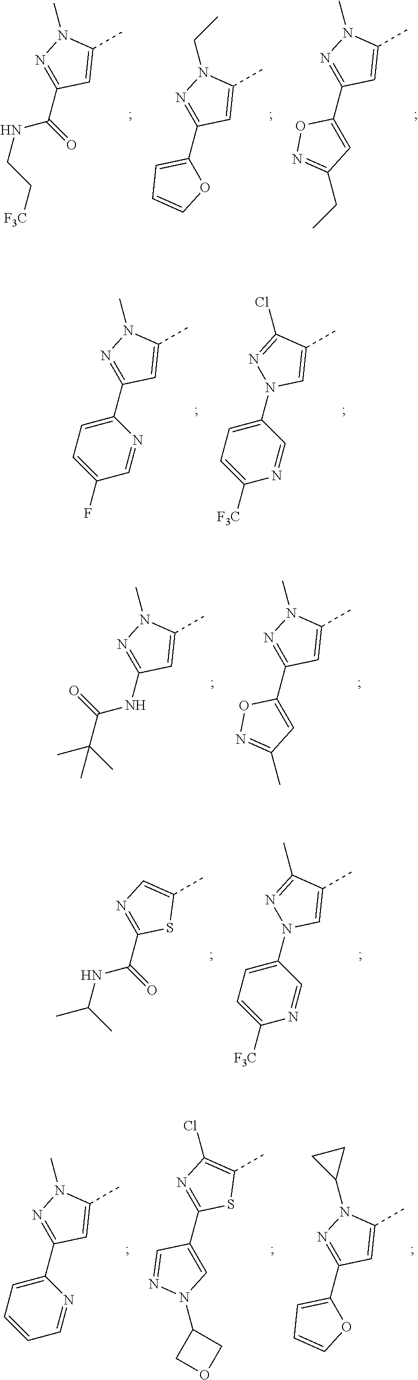

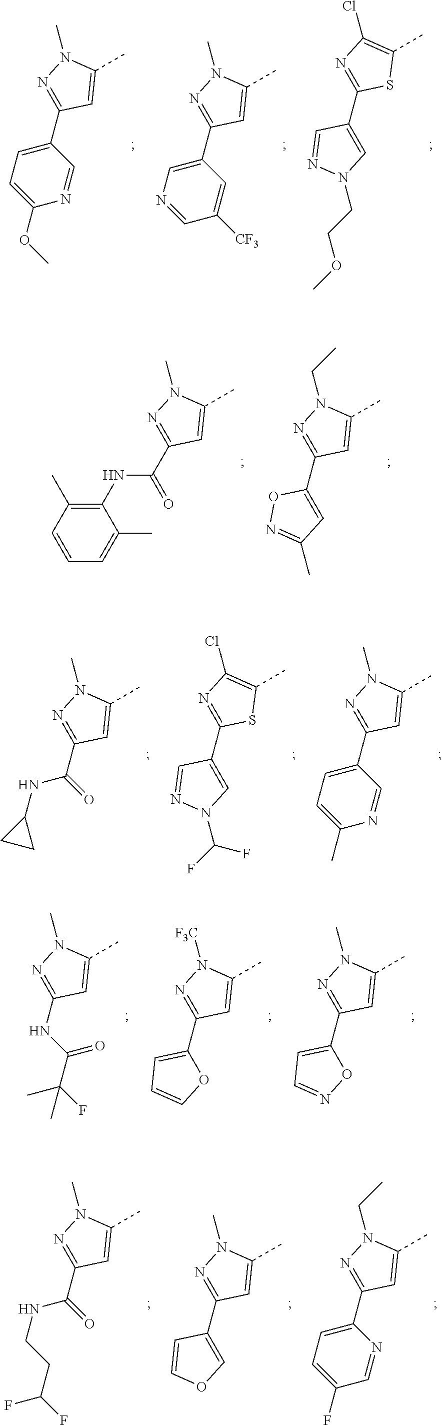

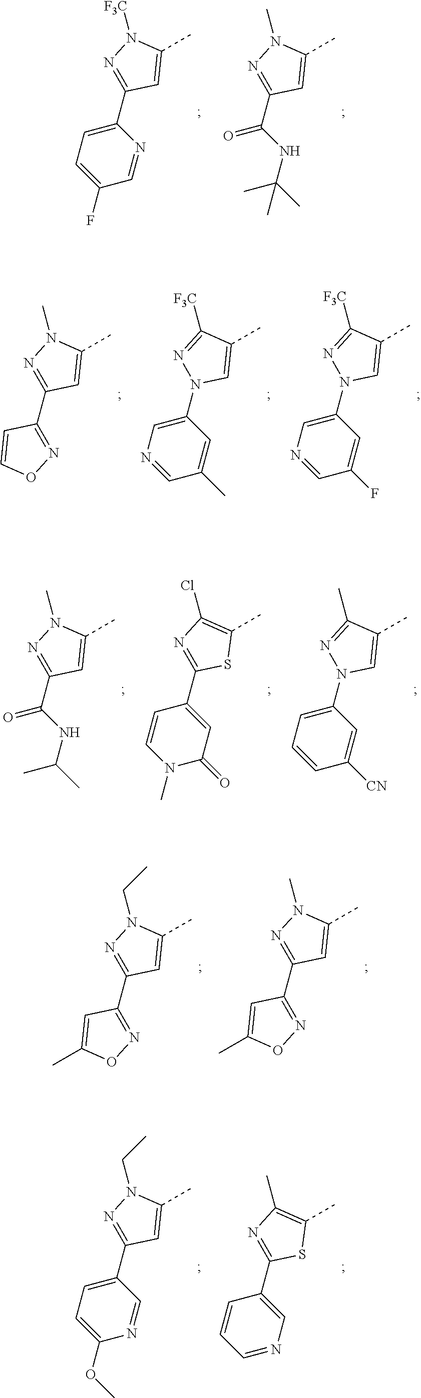

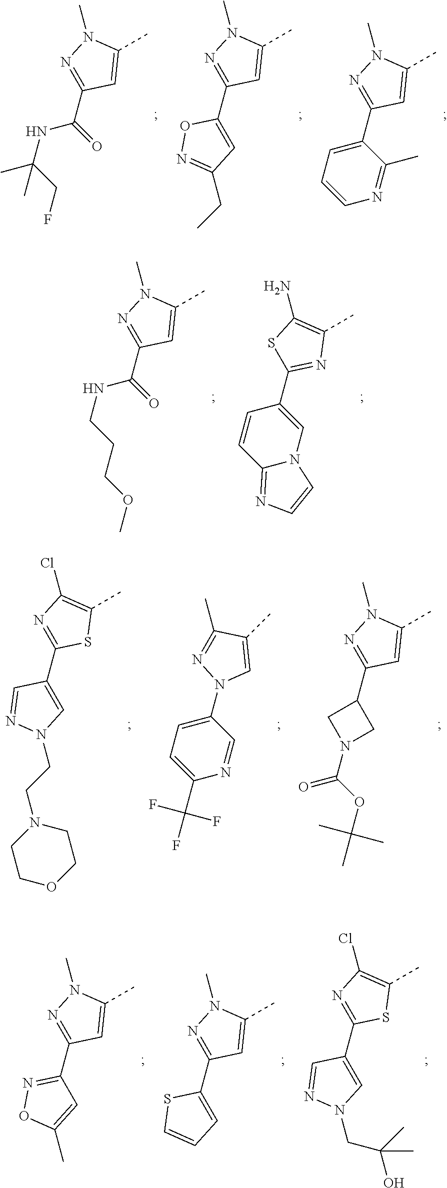

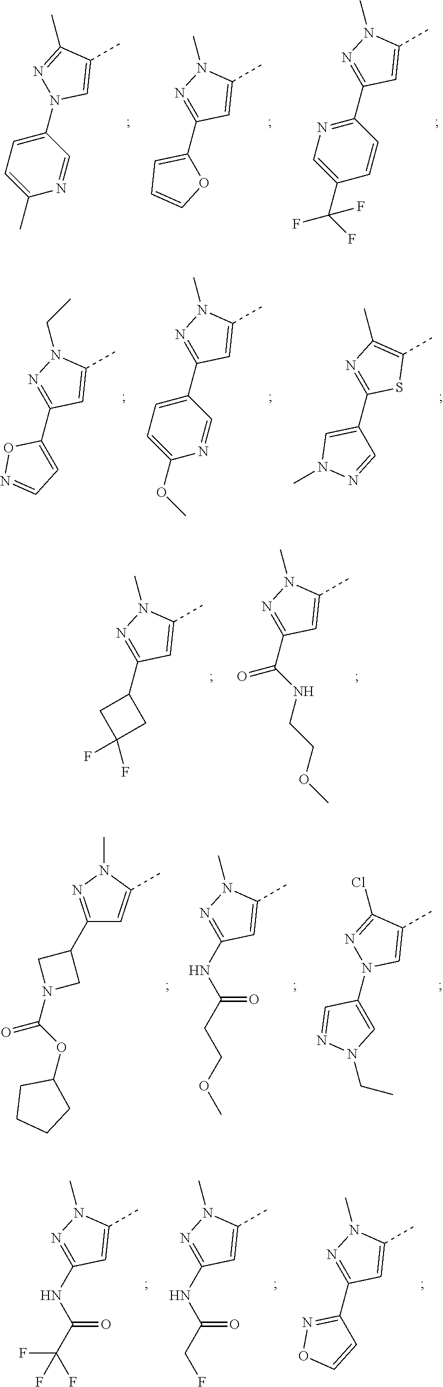

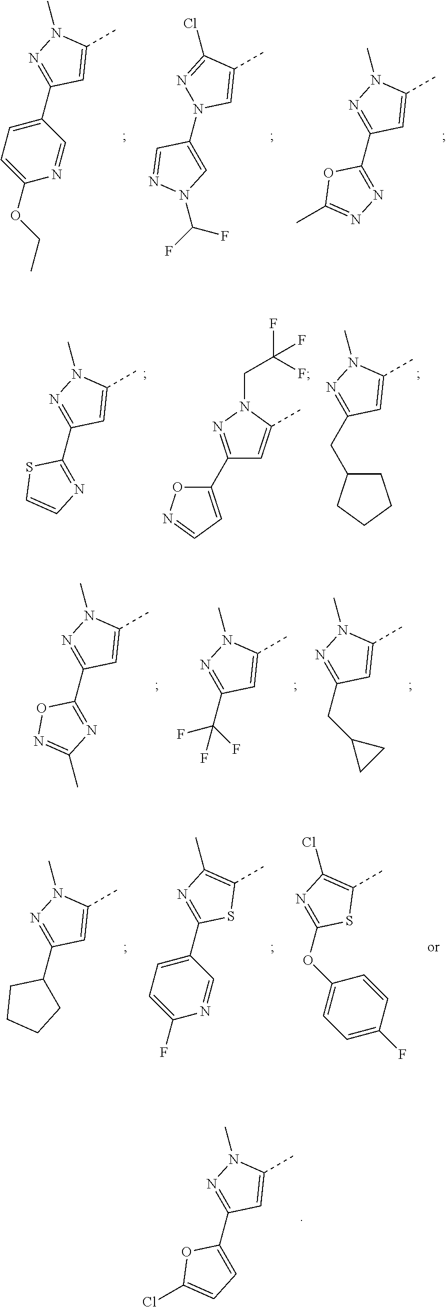

Embodiment 59. The compound of any one of Embodiments 1 to 56, or a pharmaceutically acceptable salt or stereoisomer thereof, wherein R₅ is

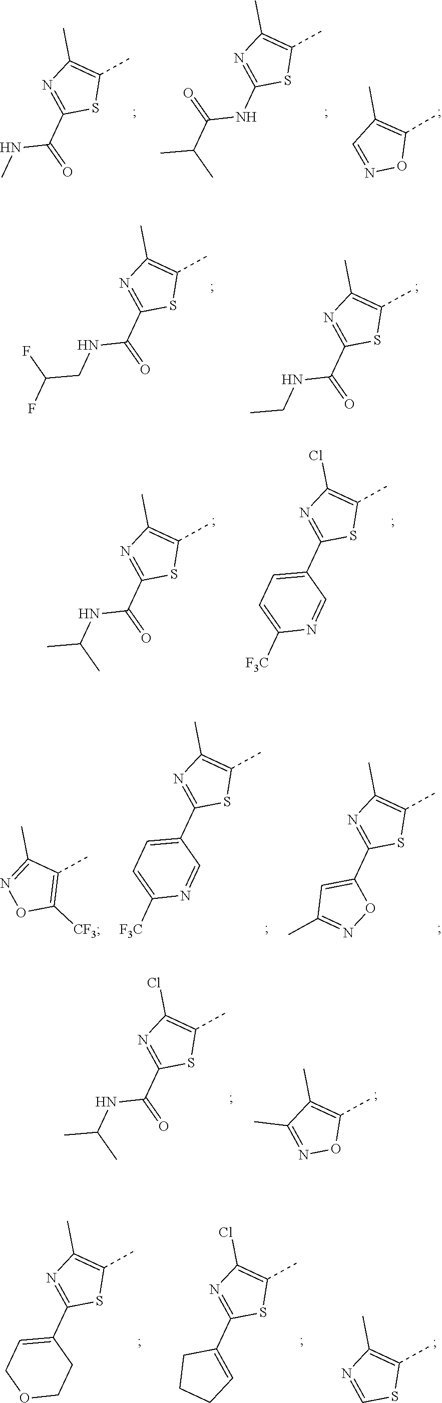

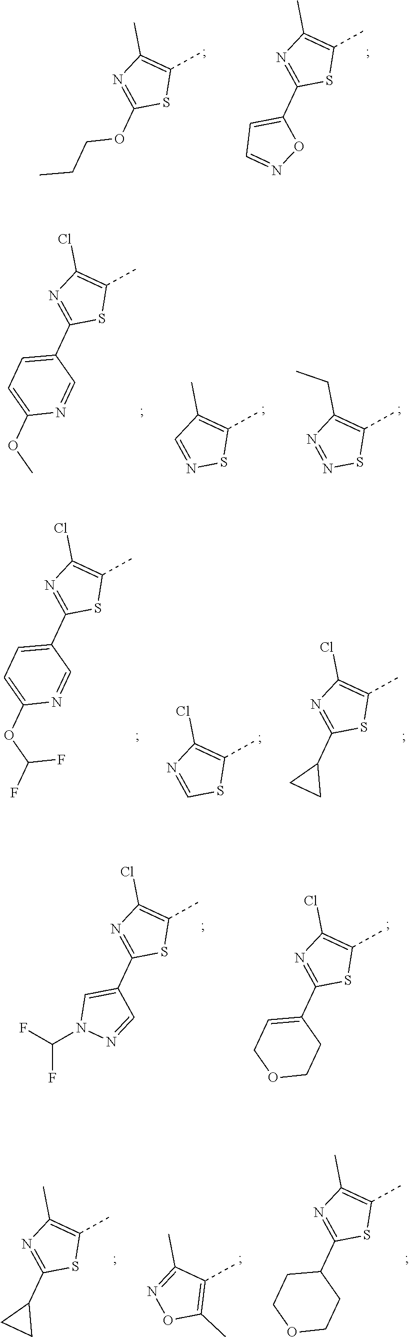

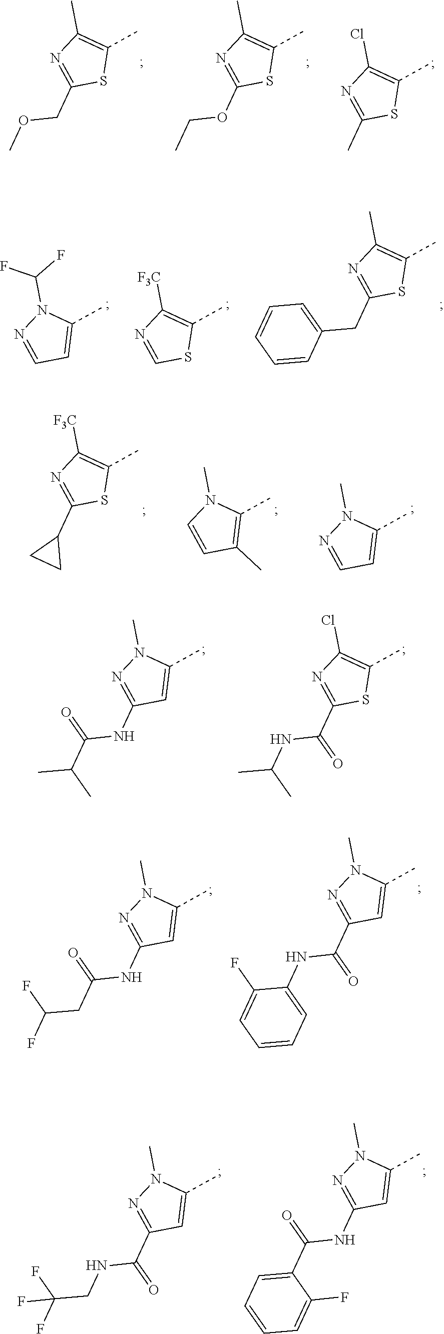

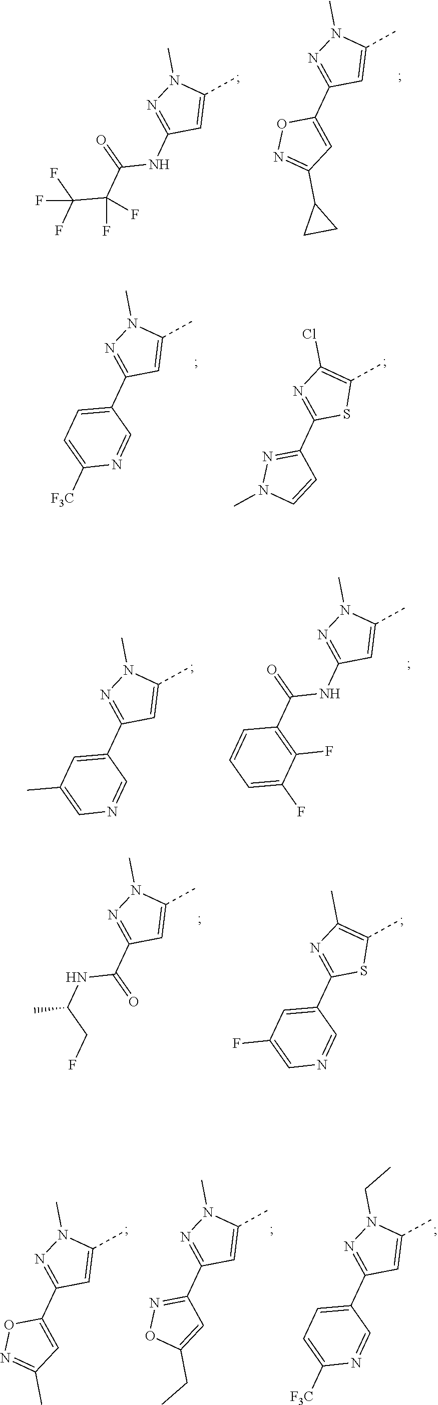

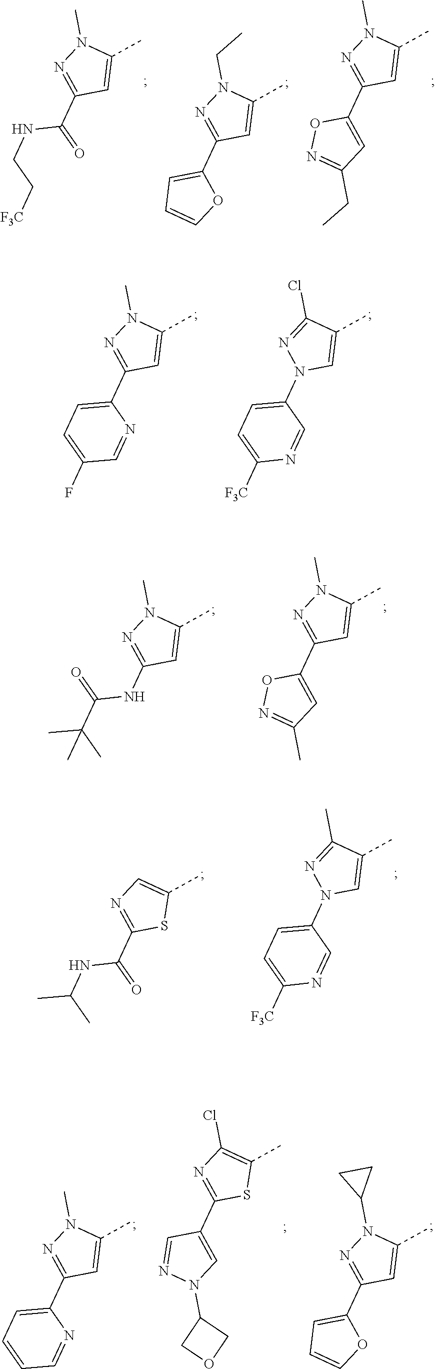

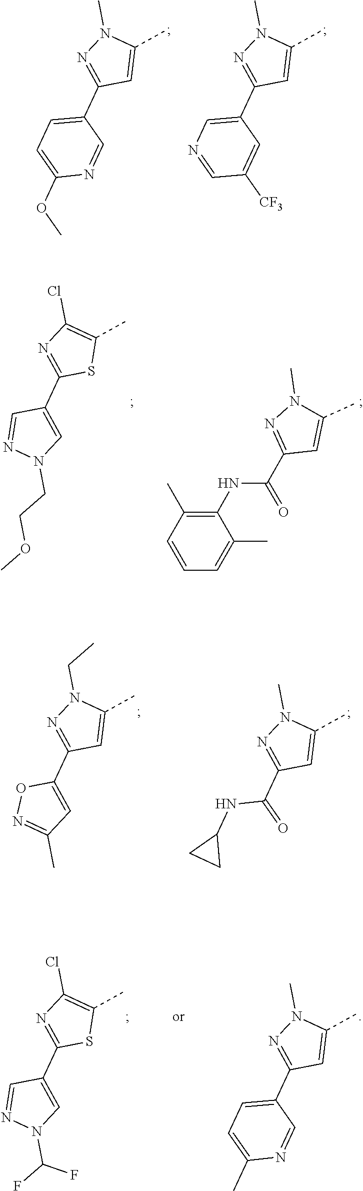

Embodiment 60. The compound of any one of Embodiments 1 to 59, or a pharmaceutically acceptable salt or stereoisomer thereof, wherein R₅ is

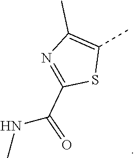

Embodiment 60A. The compound of any one of Embodiments 1 to 59, or a pharmaceutically acceptable salt or stereoisomer thereof, wherein R₅ is

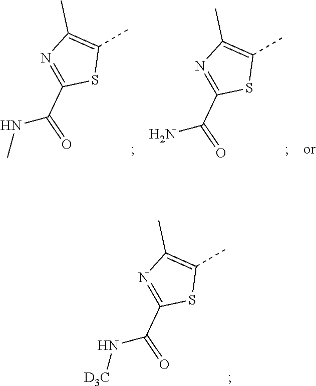

Embodiment 61. The compound of any one of Embodiments 1 to 60, or a pharmaceutically acceptable salt or stereoisomer thereof, wherein R⁷ is D, CD₃, C₁₋₆ alkyl, C₆₋₁₀ aryl-C₁₋₆ alkylene, or (5-10 membered heteroaryl)-C₁₋₆ alkylene.

Embodiment 62. The compound of any one of Embodiments 1 to 61, or a pharmaceutically acceptable salt or stereoisomer thereof, wherein R⁷ is D or methyl.

Embodiment 63. The compound of any one of Embodiments 1 to 62, or a pharmaceutically acceptable salt or stereoisomer thereof, wherein R⁷ is D.

Embodiment 64. The compound of any one of Embodiments 1 to 61, or a pharmaceutically acceptable salt or stereoisomer thereof, wherein R⁷ is CD₃.

Embodiment 65. The compound of any one of Embodiments 1 to 61, or a pharmaceutically acceptable salt or stereoisomer thereof, wherein R⁷ is methyl.

Embodiment 66. The compound of any one of Embodiments 1 to 61, or a pharmaceutically acceptable salt or stereoisomer thereof, wherein R⁷ is ethyl.

Embodiment 67. The compound of any one of Embodiments 1 to 61, or a pharmaceutically acceptable salt or stereoisomer thereof, wherein R⁷ is benzyl.

Embodiment 68. The compound of any one of Embodiments 1 to 61, or a pharmaceutically acceptable salt or stereoisomer thereof, wherein R⁷ is fluorobenzyl.

Embodiment 69. The compound of any one of Embodiments 1 to 61, or a pharmaceutically acceptable salt or stereoisomer thereof, wherein R⁷ is pyridinylmethyl.

Embodiment 70. The compound of Embodiment 1, or a pharmaceutically acceptable salt or stereoisomer thereof, wherein the compound is
N²,4-dimethyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide;
N²,4-dimethyl-N⁶—((R)-2-methyl-3-oxo-3-(((R)-11-oxo-2,3,10,11-tetrahydro-1H,5H-benzo[d]pyrazolo[1,2-a][1,2]diazepin-10-yl-10-d)amino)propyl)thiazole-2,5-dicarboxamide;
N²,4-dimethyl-N⁶—((R)-2-methyl-3-(((S)-10-methyl-11-oxo-2,3,10,11-tetrahydro-1H,5H-benzo[d]pyrazolo[1,2-a][1,2]diazepin-10-yl)amino)-3-oxopropyl)thiazole-2,5-dicarboxamide;
N²,4-dimethyl-N⁶—((R)-2-methyl-3-(((R)-10-methyl-11-oxo-2,3,10,11-tetrahydro-1H,5H-benzo[d]pyrazolo[1,2-a][1,2]diazepin-10-yl)amino)-3-oxopropyl)thiazole-2,5-dicarboxamide;
N⁵—((R)-3-(((S)-10-benzyl-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((R)-10-benzyl-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((S)-10-ethyl-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((R)-10-ethyl-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N²,4-dimethyl-N⁶—((R)-2-methyl-3-oxo-3-(((S)-11-oxo-10-(pyridin-3-ylmethyl)-2,3,10,11-tetrahydro-1H,5H-benzo[d]pyrazolo[1,2-a][1,2]diazepin-10-yl)amino)propyl)thiazole-2,5-dicarboxamide;
N²,4-dimethyl-N⁶—((R)-2-methyl-3-oxo-3-(((R)-11-oxo-10-(pyridin-3-ylmethyl)-2,3,10,11-tetrahydro-1H,5H-benzo[d]pyrazolo[1,2-a][1,2]diazepin-10-yl)amino)propyl)thiazole-2,5-dicarboxamide;
N⁵—((R)-3-(((S)-10-(4-fluorobenzyl)-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((R)-10-(4-fluorobenzyl)-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N²,4-dimethyl-N⁶—((R)-2-methyl-3-(((S)-10-(methyl-d₃)-11-oxo-2,3,10,11-tetrahydro-1H,5H-benzo[d]pyrazolo[1,2-a][1,2]diazepin-10-yl)amino)-3-oxopropyl)thiazole-2,5-dicarboxamide;
N²,4-dimethyl-N⁵—((R)-2-methyl-3-(((R)-10-(methyl-d₃)-11-oxo-2,3,10,11-tetrahydro-1H,5H-benzo[d]pyrazolo[1,2-a][1,2]diazepin-10-yl)amino)-3-oxopropyl)thiazole-2,5-dicarboxamide;
N²,4-dimethyl-N⁵—((R)-2-methyl-3-oxo-3-(((S)-11-oxo-7-(trifluoromethyl)-2,3,10,11-tetrahydro-1H,5H-benzo[d]pyrazolo[1,2-a][1,2]diazepin-10-yl)amino)propyl)thiazole-2,5-dicarboxamide;
N²,4-dimethyl-N⁶—((R)-2-methyl-3-oxo-3-(((R)-11-oxo-7-(trifluoromethyl)-2,3,10,11-tetrahydro-1H,5H-benzo[d]pyrazolo[1,2-a][1,2]diazepin-10-yl)amino)propyl)thiazole-2,5-dicarboxamide;
N²,4-dimethyl-N⁶—((R)-2-methyl-3-oxo-3-(((S)-11-oxo-7-(trifluoromethyl)-2,3,10,11-tetrahydro-1H,5H-benzo[d]pyrazolo[1,2-a][1,2]diazepin-10-yl-10-d)amino)propyl)thiazole-2,5-dicarboxamide;
N²,4-dimethyl-N⁵—((R)-2-methyl-3-oxo-3-(((R)-11-oxo-7-(trifluoromethyl)-2,3,10,11-tetrahydro-1H,5H-benzo[d]pyrazolo[1,2-a][1,2]diazepin-10-yl-10-d)amino)propyl)thiazole-2,5-dicarboxamide;
N⁵—((R)-3-(((S)-7-methoxy-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((R)-7-methoxy-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((S)-7-fluoro-11-oxo-2,3,10,11-tetrahydro-1H,5H-benzo[d]pyrazolo[1,2-a][1,2]diazepin-10-yl-10-d)amino)-2-methyl-3-oxopropyl)-N²,4-dimethylthiazole-2,5-dicarboxamide;
N⁵—((R)-3-(((R)-7-fluoro-11-oxo-2,3,10,11-tetrahydro-1H,5H-benzo[d]pyrazolo[1,2-a][1,2]diazepin-10-yl-10-d)amino)-2-methyl-3-oxopropyl)-N²,4-dimethylthiazole-2,5-dicarboxamide;
N⁵—((R)-3-(((S)-7-chloro-11-oxo-2,3,10,11-tetrahydro-1H,5H-benzo[d]pyrazolo[1,2-a][1,2]diazepin-10-yl-10-d)amino)-2-methyl-3-oxopropyl)-N²,4-dimethylthiazole-2,5-dicarboxamide; and
N⁵—((R)-3-(((R)-7-chloro-11-oxo-2,3,10,11-tetrahydro-1H,5H-benzo[d]pyrazolo[1,2-a][1,2]diazepin-10-yl-10-d)amino)-2-methyl-3-oxopropyl)-N²,4-dimethylthiazole-2,5-dicarboxamide.

Embodiment 71. The compound of Embodiment 1, wherein the compound is
N²,4-dimethyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide;
N²,4-dimethyl-N⁵—((R)-2-methyl-3-oxo-3-(((R)-11-oxo-2,3,10,11-tetrahydro-1H,5H-benzo[d]pyrazolo[1,2-a][1,2]diazepin-10-yl-10-d)amino)propyl)thiazole-2,5-dicarboxamide;
N²,4-dimethyl-N⁵—((R)-2-methyl-3-(((S)-10-methyl-11-oxo-2,3,10,11-tetrahydro-1H,5H-benzo[d]pyrazolo[1,2-a][1,2]diazepin-10-yl)amino)-3-oxopropyl)thiazole-2,5-dicarboxamide;
N²,4-dimethyl-N⁶—((R)-2-methyl-3-(((R)-10-methyl-11-oxo-2,3,10,11-tetrahydro-1H,5H-benzo[d]pyrazolo[1,2-a][1,2]diazepin-10-yl)amino)-3-oxopropyl)thiazole-2,5-dicarboxamide;
N⁵—((R)-3-(((S)-10-Benzyl-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((R)-10-benzyl-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((S)-10-Ethyl-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((R)-10-ethyl-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N²,4-dimethyl-N⁵—((R)-2-methyl-3-oxo-3-(((S)-11-oxo-10-(pyridin-3-ylmethyl)-2,3,10,11-tetrahydro-1H,5H-benzo[d]pyrazolo[1,2-a][1,2]diazepin-10-yl)amino)propyl)thiazole-2,5-dicarboxamide;
N²,4-dimethyl-N⁶—((R)-2-methyl-3-oxo-3-(((R)-11-oxo-10-(pyridin-3-ylmethyl)-2,3,10,11-tetrahydro-1H,5H-benzo[d]pyrazolo[1,2-a][1,2]diazepin-10-yl)amino)propyl)thiazole-2,5-dicarboxamide;
N⁵—((R)-3-(((S)-10-(4-Fluorobenzyl)-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((R)-10-(4-fluorobenzyl)-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N²,4-dimethyl-N⁵—((R)-2-methyl-3-(((S)-10-(methyl-d₃)-11-oxo-2,3,10,11-tetrahydro-1H,5H-benzo[d]pyrazolo[1,2-a][1,2]diazepin-10-yl)amino)-3-oxopropyl)thiazole-2,5-dicarboxamide;
N²,4-dimethyl-N⁵—((R)-2-methyl-3-(((R)-10-(methyl-d₃)-11-oxo-2,3,10,11-tetrahydro-1H,5H-benzo[d]pyrazolo[1,2-a][1,2]diazepin-10-yl)amino)-3-oxopropyl)thiazole-2,5-dicarboxamide;
N²,4-dimethyl-N⁶—((R)-2-methyl-3-oxo-3-(((S)-11-oxo-7-(trifluoromethyl)-2,3,10,11-tetrahydro-1H,5H-benzo[d]pyrazolo[1,2-a][1,2]diazepin-10-yl)amino)propyl)thiazole-2,5-dicarboxamide;
N²,4-dimethyl-N⁶—((R)-2-methyl-3-oxo-3-(((R)-11-oxo-7-(trifluoromethyl)-2,3,10,11-tetrahydro-1H,5H-benzo[d]pyrazolo[1,2-a][1,2]diazepin-10-yl)amino)propyl)thiazole-2,5-dicarboxamide;
N²,4-dimethyl-N⁶—((R)-2-methyl-3-oxo-3-(((S)-11-oxo-7-(trifluoromethyl)-2,3,10,11-tetrahydro-1H,5H-benzo[d]pyrazolo[1,2-a][1,2]diazepin-10-yl-10-d)amino)propyl)thiazole-2,5-dicarboxamide;
N²,4-dimethyl-N⁵—((R)-2-methyl-3-oxo-3-(((R)-11-oxo-7-(trifluoromethyl)-2,3,10,11-tetrahydro-1H,5H-benzo[d]pyrazolo[1,2-a][1,2]diazepin-10-yl-10-d)amino)propyl)thiazole-2,5-dicarboxamide;
N⁵—((R)-3-(((S)-7-methoxy-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((R)-7-methoxy-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((S)-7-fluoro-11-oxo-2,3,10,11-tetrahydro-1H,5H-benzo[d]pyrazolo[1,2-a][1,2]diazepin-10-yl-10-d)amino)-2-methyl-3-oxopropyl)-N²,4-dimethylthiazole-2,5-dicarboxamide;
N⁵—((R)-3-(((R)-7-fluoro-11-oxo-2,3,10,11-tetrahydro-1H,5H-benzo[d]pyrazolo[1,2-a][1,2]diazepin-10-yl-10-d)amino)-2-methyl-3-oxopropyl)-N²,4-dimethylthiazole-2,5-dicarboxamide;
N⁵—((R)-3-(((S)-7-chloro-11-oxo-2,3,10,11-tetrahydro-1H,5H-benzo[d]pyrazolo[1,2-a][1,2]diazepin-10-yl-10-d)amino)-2-methyl-3-oxopropyl)-N²,4-dimethylthiazole-2,5-dicarboxamide; and
N⁵—((R)-3-(((R)-7-chloro-11-oxo-2,3,10,11-tetrahydro-1H,5H-benzo[d]pyrazolo[1,2-a][1,2]diazepin-10-yl-10-d)amino)-2-methyl-3-oxopropyl)-N²,4-dimethylthiazole-2,5-dicarboxamide.

Embodiment 72. The compound of Embodiment 1, or a pharmaceutically acceptable salt or stereoisomer thereof, wherein the compound is

N²,4-dimethyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide; or
N²,4-dimethyl-N⁶—((R)-2-methyl-3-oxo-3-(((R)-11-oxo-2,3,10,11-tetrahydro-1H,5H-benzo[d]pyrazolo[1,2-a][1,2]diazepin-10-yl-10-d)amino)propyl)thiazole-2,5-dicarboxamide.

Embodiment 73. A compound, or a pharmaceutically acceptable salt or stereoisomer thereof, wherein the compound is
4-methyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl)amino)propyl)thiazole-2,5-dicarboxamide;
4-methyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide;
4-methyl-N⁵—((R)-2-methyl-3-oxo-3-(((R)-11-oxo-2,3,10,11-tetrahydro-1H,5H-benzo[d]pyrazolo[1,2-a][1,2]diazepin-10-yl-10-d)amino)propyl)thiazole-2,5-dicarboxamide;
4-methyl-N²-(methyl-d₃)—N⁵—((R)-2-methyl-3-oxo-3-(((S)-11-oxo-2,3,10,11-tetrahydro-1H,5H-benzo[d]pyrazolo[1,2-a][1,2]diazepin-10-yl)amino)propyl)thiazole-2,5-dicarboxamide;
4-methyl-N²-(methyl-d₃)—N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide; or
4-methyl-N²-(methyl-d₃)—N⁵—((R)-2-methyl-3-oxo-3-(((R)-11-oxo-2,3,10,11-tetrahydro-1H,5H-benzo[d]pyrazolo[1,2-a][1,2]diazepin-10-yl-10-d)amino)propyl)thiazole-2,5-dicarboxamide.

Embodiment 74. A compound of Formula (Ia) or a pharmaceutically acceptable salt or stereoisomer thereof,

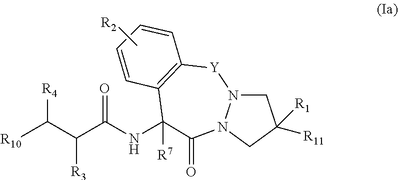

wherein:
Y is CH₂ or C═O;
R₁ is H, C₁-C₆alkyl or halo;
R₂ is C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₄ is H, C₁-C₆alkyl or C₁-C₆alkyl-phenyl;
R₁₀ is —NHC(═O)R₅, —C(═O)NHR, or a 9 or 10 membered bicyclic heteroaryl having 1 to 4 heteroatoms as ring members each independently selected from N, O and S, wherein the bicyclic heteroaryl is unsubstituted or the bicyclic heteroaryl is substituted with one or more R₆;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by one or more D or C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo;
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy; and
xxi) —C(═O)NH₂;
each R₆ is independently selected from C₁-C₆alkyl, C₁-C₆alkoxy, C₁-C₆haloalkyl, cyano and halo;
R₁₁ is H, C₁-C₆alkyl or halo; or
R₁ and R₁₁ together with the carbon atom to which they are attached, may form a 3 to 6 membered carbocyclic ring; and
R⁷ is H.

Embodiment 74A. The compound of Embodiment 74, wherein
Y is CH₂ or C═O;
R₁ is H, C₁-C₆alkyl or halo;
R₂ is C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₄ is H, C₁-C₆alkyl or C₁-C₆alkyl-phenyl;
R₁₀ is —NHC(═O)R₅, —C(═O)NHR, or a 9 or 10 membered bicyclic heteroaryl having 1 to 4 heteroatoms as ring members each independently selected from N, O and S, wherein the bicyclic heteroaryl is unsubstituted or the bicyclic heteroaryl is substituted with one or more R₆;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo; and
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy;
each R₆ is independently selected from C₁-C₆alkyl, C₁-C₆alkoxy, C₁-C₆haloalkyl, cyano and halo;
R₁₁ is H, C₁-C₆alkyl or halo; or
R₁ and R₁₁ together with the carbon atom to which they are attached, may form a 3 to 6 membered carbocyclic ring; and
R⁷ is H.

Depending on the choice of the starting materials and procedures, the compounds can be present in the form of one of the possible isomers or as mixtures thereof, for example as pure optical isomers, or as isomer mixtures, such as racemates and diastereoisomer mixtures, depending on the number of asymmetric carbon atoms, i.e., the present disclosure is meant to include all such possible isomers, including racemic mixtures, diastereomeric mixtures and optically pure forms. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66,2 (1977), each of which is incorporated herein by reference in its entirety.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the present disclosure include, for example, isotopes of hydrogen.

It will be recognized that some variation of natural isotopic abundance occurs in a synthesized compound depending upon the origin of chemical materials used in the synthesis. Thus, a preparation of a compound disclosed herein will inherently contain small amounts of deuterated isotopologues. The concentration of naturally abundant stable hydrogen and carbon isotopes, notwithstanding this variation, is small and immaterial as compared to the degree of stable isotopic substitution of compounds of this disclosure. See, for instance, Wada, E et al., Scikagaku, 1994, 66:15; Gannes, L Z et al., Comp Biochem Physiol Mol Integr Physiol, 1998, 119:725.

In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Also, unless otherwise stated, when a position is designated specifically as “D” or “deuterium,” the position is understood to have deuterium at an abundance that is at least 3000 times greater than the natural abundance of deuterium, which is 0.015% (i.e., at least 45% incorporation of deuterium).

In an embodiment, any atom not designated as deuterium in the compounds provided herein is present at its natural isotopic abundance, and wherein for each site designated as deuterium, deuterium incorporation is at least 40%, 50%, 60%, 70%, 80%, or 90%. In another embodiment, any atom not designated as deuterium in the compounds provided herein is present at its natural isotopic abundance, and wherein for each site designated as deuterium, deuterium incorporation is at least 80%. In still another embodiment, any atom not designated as deuterium in the compounds provided herein is present at its natural isotopic abundance, and wherein for each site designated as deuterium, deuterium incorporation is at least 90%. In an embodiment, any atom not designated as deuterium in Formula (I) provided herein is present at its natural isotopic abundance, and wherein for each site designated as deuterium, deuterium incorporation is at least 40%, 50%, 60%, 70%, 80%, or 90%. In another embodiment, any atom not designated as deuterium in Formula (I) provided herein is present at its natural isotopic abundance, and wherein for each site designated as deuterium, deuterium incorporation is at least 80%. In yet another embodiment, any atom not designated as deuterium in Formula (I) provided herein is present at its natural isotopic abundance, and wherein for each site designated as deuterium, deuterium incorporation is at least 90%.

In an embodiment, any atom not designated as deuterium in Example 1 provided herein is present at its natural isotopic abundance, and wherein for each site designated as deuterium, deuterium incorporation is at least 40%, 50%, 60%, 70%, 80%, or 90%. In another embodiment, any atom not designated as deuterium in Example 1 provided herein is present at its natural isotopic abundance, and wherein for each site designated as deuterium, deuterium incorporation is at least 80%. In still another embodiment, any atom not designated as deuterium in Example 1 provided herein is present at its natural isotopic abundance, and wherein for each site designated as deuterium, deuterium incorporation is at least 90%.

In an embodiment, any atom not designated as deuterium in Examples 2, 29, or 30 provided herein is present at its natural isotopic abundance, and wherein for each site designated as deuterium, deuterium incorporation is at least 40%, 50%, 60%, 70%, 80%, or 90%. In another embodiment, any atom not designated as deuterium in Examples 2, 29, or 30 provided herein is present at its natural isotopic abundance, and wherein for each site designated as deuterium, deuterium incorporation is at least 80%. In yet another embodiment, any atom not designated as deuterium in Examples 2, 29, or 30 provided herein is present at its natural isotopic abundance, and wherein for each site designated as deuterium, deuterium incorporation is at least 90%.

Further, incorporation of certain isotopes, particularly deuterium (i.e., 2H or D) may afford certain therapeutic advantages resulting from greater metabolic stability, for example increased in vivo half-life, reduced dosage requirements, an improvement in therapeutic index, tolerability, and/or lower frequency of epimerization. It is understood that deuterium in this context is regarded as a substituent of a compound of the present disclosure. The concentration of deuterium may be defined by the isotopic enrichment factor. The term “isotopic enrichment factor” as used herein means the ratio between the isotopic abundance and the natural abundance of a specified isotope. If a substituent in a compound of this disclosure is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the present disclosure include isotopes of hydrogen, carbon, nitrogen, oxygen, phosphorous, fluorine, and chlorine, such as ³H, ¹¹C, ¹³C, ¹⁴C, ¹⁵N, ¹⁸F ³¹P, ³²P, ³⁵S, ³⁶Cl, ¹²³I, ¹²⁴I, ¹²⁵I respectively. Accordingly, it should be understood that the disclosure includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present disclosure can generally be prepared by conventional techniques known to those skilled in the art or by processes analogous to those described in the accompanying Examples and Preparations using an appropriate isotopically-labeled reagents in place of the non-labeled reagent previously employed.).

By way of example, compounds of the present disclosure can exist in a deuterated form as shown below:

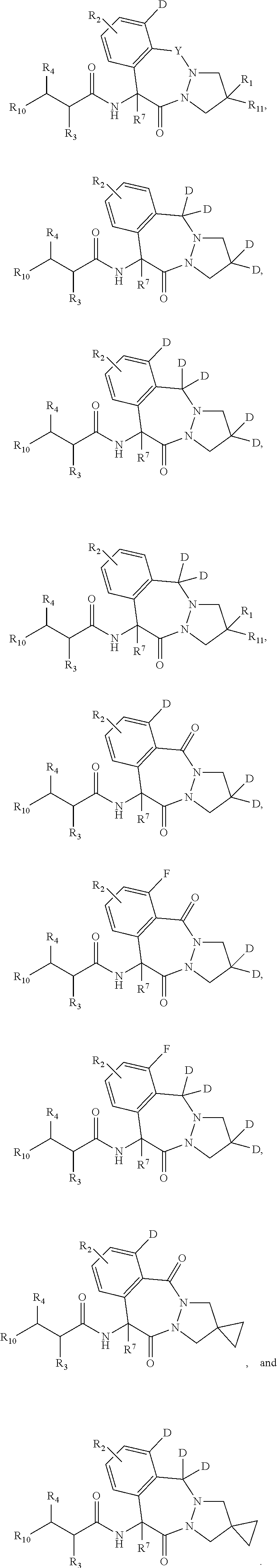

In an embodiment of the deuterated formulae above, R⁷ is D. In another embodiment of the deuterated formulae above, R⁷ is methyl.

Pharmaceutically acceptable solvates in accordance with the disclosure include those wherein the solvent of crystallization may be isotopically substituted, e.g., D₂O, ^{d6}-acetone, ^{d6}-DMSO.

Compounds of the disclosure that contain groups capable of acting as donors and/or acceptors for hydrogen bonds may be capable of forming co-crystals with suitable co-crystal formers. These co-crystals may be prepared from compounds of the disclosure by known co-crystal forming procedures. Such procedures include grinding, heating, co-subliming, co-melting, or contacting in solution compounds of the disclosure with the co-crystal former under crystallization conditions and isolating co-crystals thereby formed. Suitable co-crystal formers include those described in WO 2004/078163. Hence the disclosure further provides co-crystals comprising a compound of the disclosure.

Furthermore, the compounds of the present disclosure, including their salts, can also be obtained in the form of their hydrates, or include other solvents used for their crystallization. The compounds of the present disclosure may inherently or by design form solvates with pharmaceutically acceptable solvents (including water); therefore, it is intended that the disclosure embrace both solvated and unsolvated forms. The term “solvate” refers to a molecular complex of a compound of the present disclosure (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

Any asymmetric atom (e.g., carbon or the like) of the compound(s) of the present disclosure can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or(S)-configuration. Substituents at atoms with unsaturated double bonds may, if possible, be present in cis-(Z)- or trans-(E)-form.

When the compounds described herein contain an asymmetric carbon, unless otherwise indicated, the compounds can be any of the possible stereoisomers. In some embodiments, the compounds provided herein have the (R)-configuration. In other embodiments, the compounds have the(S)-configuration. In compounds with more than one asymmetric carbon atoms, each of the carbon atoms in the compound may be independently (R) or(S), unless otherwise indicated. In compounds with a single asymmetric carbon, the stereochemistry of the asymmetric carbon can be (R) or(S). In compounds with two asymmetric carbon atoms, the stereochemistry of the carbon atoms can each be independently (R) or(S) so the configuration of the carbon atoms can be (R) and (R), (R) and(S); (S) and (R), or(S) and(S). In compounds with three asymmetric carbon atoms, the stereochemistry each of the three carbon atoms can each be independently (R) or(S) so the configuration of the carbon atoms can be (R), (R) and (R); (R), (R) and(S); (R), (S) and (R); (R), (S) and(S); (S), (R) and (R); (S), (R) and(S); (S), (S) and (R); or(S), (S) and(S).

Accordingly, as used herein a compound of the present disclosure can be in the form of one of the possible isomers, rotamers, atropisomers, tautomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization. Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present disclosure into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

### Processes for Making Compounds

General procedures for preparing compounds of the present disclosure are described herein. In the reactions described, reactive functional groups, for example hydroxy, amino, imino or carboxy groups, where these are desired in the final product, may be protected to avoid their unwanted participation in the reactions. Within the scope of this text, only a readily removable group that is not a constituent of the particular desired end product of the compounds of the present disclosure is designated a “protecting group,” unless the context indicates otherwise. The protection of functional groups by such protecting groups, the protecting groups themselves, and their cleavage reactions are described for example in standard reference works, such as J. F. W. McOmic, “Protective Groups in Organic Chemistry” Plenum Press, London and New York 1973, in T. W. Greene and P. G. M. Wuts, “Protective Groups in Organic Synthesis” Third edition, Wiley, New York 1999.

All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed.

### Methods of Synthesizing Compounds

Agents of the disclosure may be prepared by a reaction sequence shown in the reaction schemes of the experimental part (see hereinbelow).

Typically, the compounds of the disclosure may be prepared according to the Schemes 1-4 provided infra. Compounds of the present disclosure were made by processes described herein and as illustrated in the Examples. The combination of various building blocks and intermediates described herein can be applied to yield compounds of the disclosure. Non-limiting examples of synthetic schemes used to make compounds of the present disclosure is illustrated in Schemes 1 to 4. Further guidance can be found in the examples section.

Compounds of Formula (II) can be prepared as outlined in Scheme 1.

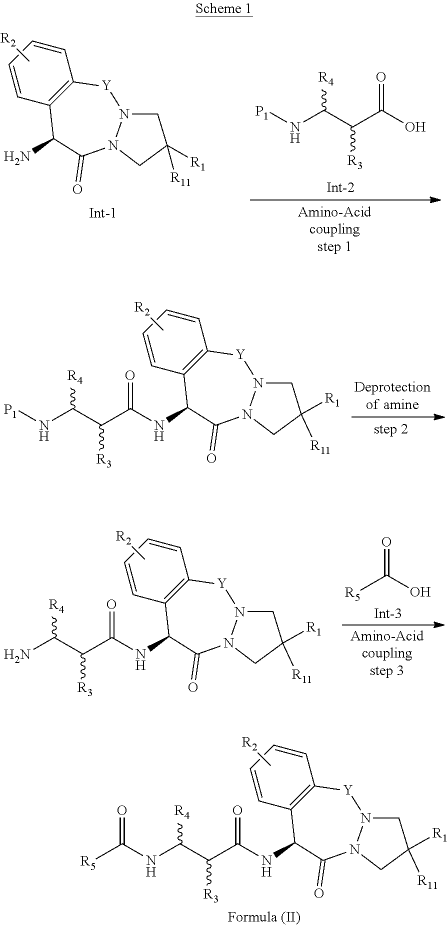

An amide of Int-1 with the corresponding N-protected β-amino acids (Int-2) can be achieved using various coupling reagents or conditions (E. Valeur, M. Bradley, Chem. Soc. Rev. 2009, 38, 606-631; A. El-Faham, F. Albericio, Chem. Rev. 2011, 111, 6557-6602). After removal of the protecting group (T. W. Greene and P. G. M. Wuts, “Protective Groups in Organic Synthesis” Third edition, Wiley, New York 1999) such as Boc or Cbz in the formed amides, the released amine intermediate can be coupled with various acid building blocks (Int-3) to provide the final compounds of Formula (II).

Similarly, compounds of Formula (III) can be prepared as outlined in Scheme 2.

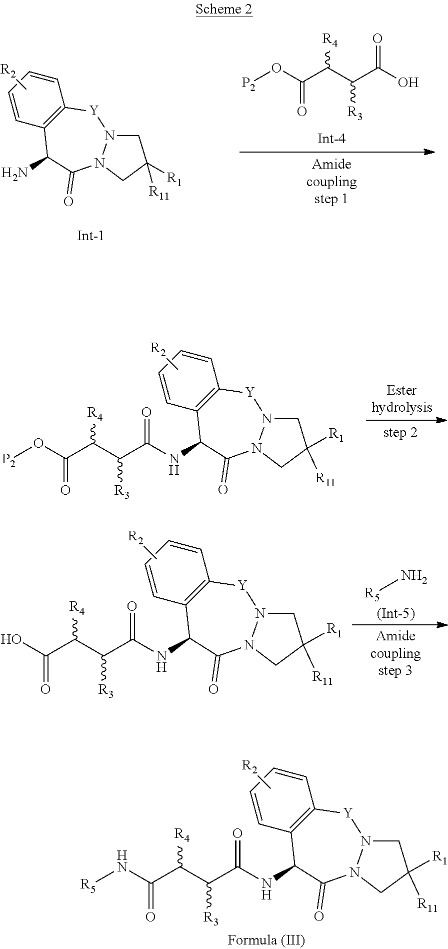

Similar to the preparation of the compounds of Formula (II), the compounds of Formula (III) can be achieved by amide coupling between amines (Int-1), but in this case various mono-protected succinates (Int-4) are used as acid partners. The chiral succinates intermediates (Int-4) can be prepared in enantiopure form by various methods including asymmetric hydrogenation of α-substituted acrylic acids using chiral catalysts (e.g., P. M. Donate, et al., Tetrahedron:Asymmetry 2003, 14, 3253-3256) or by Evans method utilizing chiral oxazolidine auxiliary (D. A. Evans, et al., J. Org. Chem. 1999, 64, 6411-6417). Alternatively, such chiral acids can be prepared also by chiral resolution (J. M. Keith, et al., Adv. Synth. Catal. 2001, 343, 5-26) using chiral amines or enzymes, by dynamic kinetic resolution or chiral separation using preparative chiral chromatography methods. The formed amide ester intermediates then undergo ester hydrolysis and the obtained acid intermediates can be coupled with aliphatic or aromatic amines to provide the final products of Formula (III).

The required chiral amine intermediates Int-1 wherein Y is CH₂ can be prepared as outlined in Scheme 3.

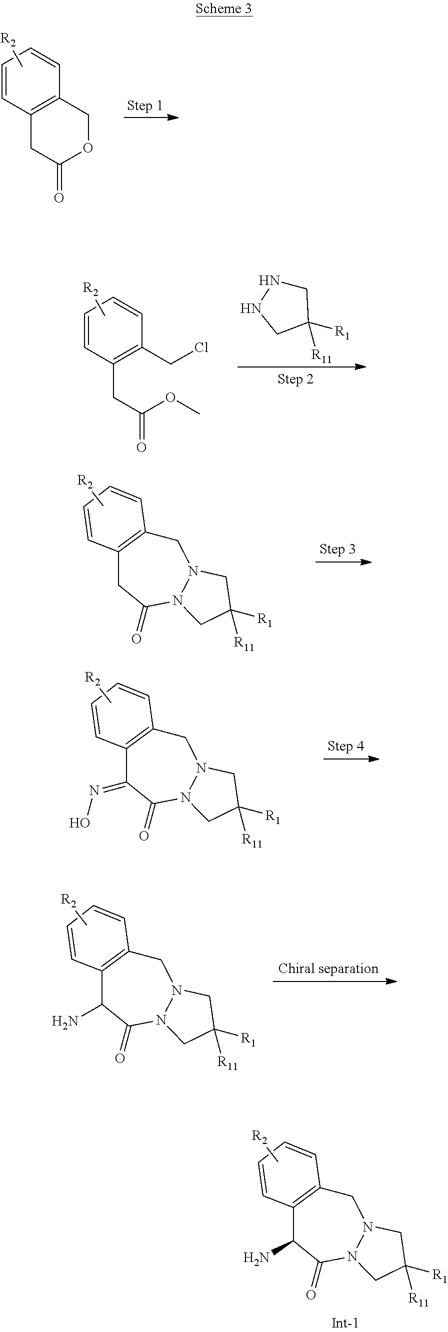

The tricyclic core is prepared by cyclization of 2-(2-(halomethyl)phenyl) acetates (prepared from the corresponding isochroman-3-ones-U.S. Pat. No. 6,048,998) with pyrazolidines (E. E. Boros, F. Bouvier, S. Randhawa, M. H. Rabinowitz, J. Heterocycl. Chem. 2001, 38, 613-616). The required primary amine can be introduced to such compounds by several ways. Such molecules can be transformed into α-bromo-derivatives that undergo a nucleophilic substitution with an azide which can then be reduced into the primary amine (e.g., WO 2015/160772). Other possibility to introduce the azide is to employ a one-step sequence utilizing the azidation of the corresponding enolate with 2,4,6-triisopropylbenzenesulfonyl azide (e.g., C. V. C. Prasad et al. Bioorg. Med. Chem. Lett. 2007, 17, 4006-4011) or a copper-catalyzed azidation (S.-E. Suh, et al., J. Am. Chem. Soc. 2020, 142, 11388-11393). Alternatively, as shown in scheme 3, the amine can also be introduced by the formation of an oxime and its reduction (F. Hoffmann-Emery, et al., Tet. Lett. 2009, 50, 6380-6382). The enantiomerically pure amine can be obtained either by chiral resolution, by formation of separable and cleavable diastereomeric mixture (F. Hoffmann-Emery, R. et al., 2009) or preparative chiral chromatography method.

Intermediate 1 (Int-1) wherein Y is C(O) can be prepared according to Scheme 4:

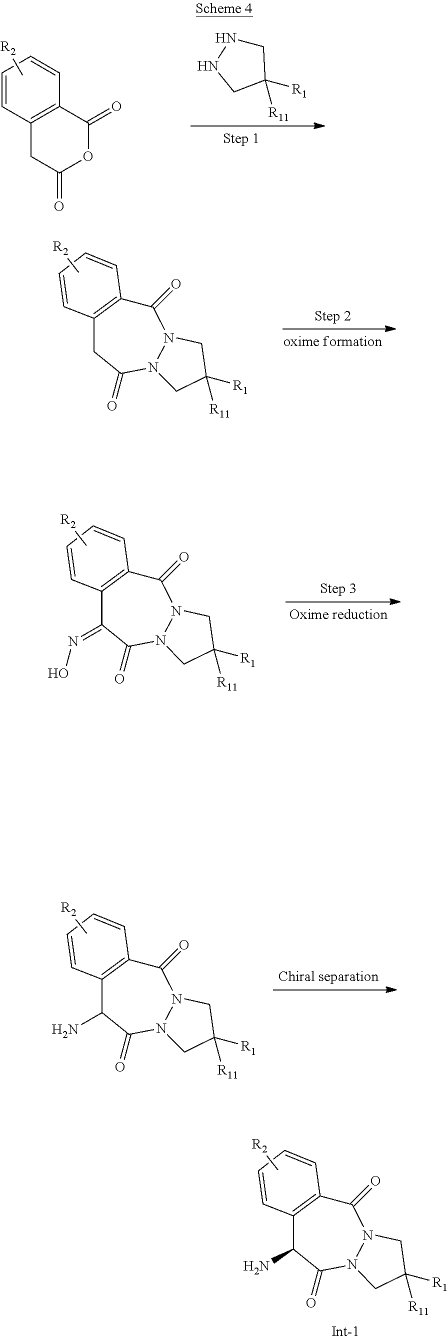

The oxo-tricycles can be made analogous to the synthesis of tricycles described in Scheme 3 if isochromane-1,3-diones instead of 2-(2-(halomethyl)phenyl) acetates are used in the cyclization with pyrazolidines. Alternatively, the Int-1 from Scheme 3 can be oxidized with RuO₂ (A. G. Schultz, et al., J. Org. Chem. 1998, 63, 7795-7804) to directly provide the Int-1 wherein Y is C(O). Chiral separation can also be performed as described in Scheme 3.

### Administration and Pharmaceutical Compositions

For the therapeutic uses of compounds of the present disclosure, such compounds are administered either alone or as part of a pharmaceutical composition. Accordingly, in another aspect of the present disclosure provides a pharmaceutical composition, which comprises a compound of the present disclosure, or pharmaceutically acceptable salt or stereoisomer thereof, and one or more pharmaceutically acceptable carriers. In a further embodiment, the composition comprises at least two pharmaceutically acceptable carriers, such as those described herein. The pharmaceutical composition can be formulated for particular routes of administration such as oral administration, parenteral administration (e.g., by injection, infusion, transdermal or topical administration), and rectal administration. Topical administration may also pertain to inhalation or intranasal application. In certain embodiments, the pharmaceutical composition comprising a compound of the present disclosure can be formulated for intramuscularly, intravenously, subcutaneously, orally, pulmonary, intrathecally, topically or intranasally administration.

The pharmaceutical compositions of the present disclosure can be made up in a solid form (including without limitation capsules, tablets, pills, granules, powders or suppositories), or in a liquid form (including without limitation solutions, suspensions or emulsions). Tablets may be either film coated or enteric coated according to methods known in the art.

Suitable compositions for oral administration include a compound of the present disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable carriers/excipients which are suitable for the manufacture of tablets. These carriers/excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g., intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present disclosure or pharmaceutical composition thereof for use in a subject (e.g., human) is typically administered orally or parenterally at a therapeutic dose of less than or equal to about 100 mg/kg. When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated.

The above-cited dosage properties are demonstrable in vitro and in vivo tests using advantageously mammals, e.g., mice, rats, dogs, monkeys or isolated organs, tissues and preparations thereof. The compounds of the present disclosure can be applied in vitro in the form of solutions, e.g., aqueous solutions, and in vivo either enterally, parenterally, advantageously intravenously, e.g., as a suspension or in aqueous solution.

Certain aspects and examples of the pharmaceutical compositions of the present disclosure are provided below. It will be recognized that features specified in each embodiment may be combined with other specified features to provide further embodiments of the present disclosure.

In an aspect, provided herein is a pharmaceutical composition comprising a compound of the present disclosure, or a pharmaceutically acceptable salt or stereoisomer thereof, and one or more pharmaceutically acceptable carriers.

In an embodiment, the pharmaceutical composition comprises one or more additional therapeutic agents.

### Pharmacology and Utility

The compounds of the disclosure, in free form or in pharmaceutically acceptable salt form, exhibit valuable pharmacological properties, e.g., inhibition of cellular levels of SPPL2a, as indicated by the in vitro tests provided herein, and are therefore indicated for therapy or for use as research chemicals, e.g., as tool compounds.

Accordingly, the compounds of the disclosure may generally be useful in the treatment of an indication involving for example cells expressing high level of CD74 and/or cells involved in class II dependent antigen presentation. In addition, the compounds of the disclosure may be useful in treating autoimmune diseases and/or disorders. In particular, the compounds of the disclosure may be useful in the treatment and/or prevention of pemphigus vulgaris, pemphigus foliaceus, Sjogren's disease, systemic lupus erythematosus (SLE), arthritis, myasthenia gravis, Hashimoto thyroiditis, thrombocytopenia purpura, myocarditis, atopic dermatitis, Goodpasture syndrome, multiple sclerosis (MS) or type I diabetes.

Furthermore, the compounds of the disclosure may be useful in the prevention of rejection in clinical/surgical transplantation procedures of solid organs, tissues or cell populations such as stem cells. Moreover, compounds of the disclosure might be useful in treating and/or preventing both acute and chronic graft versus host disease (GvHD) associated with transplantation of solid organs, tissues or cell populations. Compounds of the disclosure might further be used prophylactically, e.g., as induction therapy, to prepare the host prior to transplantation of solid organs, tissues or cell populations; or compounds of the disclosure might further be used therapeutically after transplantation of solid organs, tissues or cell populations. Non-limiting examples of transplantations are kidney transplantation, heart transplantation (acute or chronic), and bone narrow transplantation. Moreover, compounds of this disclosure might be useful in the treatment of a donor prior to the donation of organs, tissues or cells.

Additionally, compounds of the disclosure might be useful in the treatment of lymphomas in particular arising from modified B cells expressing high levels of CD74, such as Hodgkin's lymphoma, follicular lymphoma, Marginal Zone Lymphoma (MZL), Chronic Lymphocytic Leukemia (CLL), Small Lymphocytic Lymphoma (SLL), Mantle Cell Lymphoma (MCL), Diffuse Large B-Cell Lymphoma (DLBCL), Lymphoplasmacytic Lymphoma, non-Hodgkin's lymphoma (NHL), Burkitt Lymphoma (BL) and multiple myeloma (MM).

Certain aspects and examples of the use of compounds of the present disclosure and pharmaceutical compositions of the present disclosure are provided below. It will be recognized that features specified in each embodiment may be combined with other specified features to provide further embodiments of the present disclosure.

In an aspect, provided herein is a method of treating a disease or disorder associated with the activity of signal peptide peptidase like protease 2a (SPPL2a), wherein the method comprises administering to a subject in need of such treatment a therapeutically effective amount of a compound of the present disclosure, or a pharmaceutically acceptable salt.

In an embodiment, the disease or disorder is selected from NMDAR encephalitis, Neuromyelitis Optica Spectrum Disorder (NMOSD), and myelin-oligodendrocyte glycoprotein (MOG) spectrum disorder (MOGSD). In another embodiment, the disease or disorder is a myocardial disorder. In yet another embodiment, the disease or disorder is Spondyloarthritis (SpA). In still another embodiment, the disease or disorder is vasculitis.

In another embodiment, the disease or disorder is lupus. In another embodiment, the disease or disorder is cutaneous lupus.

In an embodiment of the methods, the method comprises treating systemic lupus erythematosus (SLE) in a subject in need thereof comprising administering to the subject, N²,4-dimethyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide, or a pharmaceutically acceptable salt thereof.

In an embodiment of the methods, the method comprises treating psoriasis in a subject in need thereof comprising administering to the subject, N²,4-dimethyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide, or a pharmaceutically acceptable salt thereof.

In an embodiment of the methods, the method comprises treating systemic lupus erythematosus (SLE) in a subject in need thereof comprising administering to the subject, 4-methyl-N²-(methyl-d₃)—N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide, or a pharmaceutically acceptable salt thereof.

In an embodiment of the methods, the method comprises treating psoriasis in a subject in need thereof comprising administering to the subject, 4-methyl-N-(methyl-d₃)—N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide, or a pharmaceutically acceptable salt thereof.

In an embodiment of the methods, the method comprises treating psoriasis in a subject in need thereof comprising administering to the subject, N²,4-dimethyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl)amino)propyl)thiazole-2,5-dicarboxamide, or a pharmaceutically acceptable salt thereof.

In yet another aspect, provided herein is a use of a compound provided herein, or a pharmaceutically acceptable salt thereof, in the manufacture of a medicament for the treatment of a disease or disorder associated with the activity of signal peptide peptidase like protease 2a (SPPL2a).

In still another aspect, provided herein is a use of a compound provided herein, or a pharmaceutically acceptable salt thereof, in the treatment of a disease or disorder associated with the activity of signal peptide peptidase like protease 2a (SPPL2a).

In an aspect, provided herein is a compound of the present disclosure, or a pharmaceutically acceptable salt thereof, for use in the treatment of a disease or disorder associated with the activity of signal peptide peptidase like protease 2a (SPPL2a).

In another aspect, provided herein is a method of treating an autoimmune disease in a subject in need thereof, wherein the method comprises administering to the subject a therapeutically effective amount of a compound of the present disclosure, or a pharmaceutically acceptable salt thereof.

In another embodiment, the autoimmune disease is cutaneous lupus.

In another embodiment, the autoimmune disease is autoimmune encephalomyelitis. In an embodiment, the autoimmune disease is Sjogren's disease, systemic lupus erythematosus (SLE), arthritis (such as rheumatoid arthritis and psoriatic arthritis), lupus nephritis, systemic sclerosis, multiple sclerosis (MS), autoimmune hepatitis, uveitis, pemphigus vulgaris, pemphigus foliaceus, myasthenia gravis, Hashimoto thyroiditis, thrombocytopenia purpura, myocarditis, atopic dermatitis, Goodpasture syndrome, or type I diabetes. In another embodiment, the autoimmune disease is cutaneous lupus. In an embodiment, the arthritis is juvenile arthritis.

In another embodiment, the autoimmune disease is multiple sclerosis (MS), Sjogren's disease, psoriasis, arthritis (such as rheumatoid arthritis and psoriatic arthritis), lupus nephritis or systemic sclerosis. In still another embodiment, the autoimmune disease is a neurological autoimmune disease. In an embodiment, the autoimmune disease is an autoantibody-mediated encephalitis syndrome. In an embodiment, the arthritis is juvenile arthritis. In an embodiment, the arthritis is osteoarthritis. In an embodiment, the arthritis is rheumatoid arthritis.

In yet another embodiment, the autoimmune disease is multiple sclerosis (MS).

In an embodiment, the autoimmune disease is autoimmune myositis, dermatomyositis, polymyositis, or juvenile myositis.

In still another aspect, provided herein is a use of a compound provided herein, or a pharmaceutically acceptable salt thereof, in the manufacture of a medicament for the treatment of an autoimmune disease.

In an aspect, provided herein is a use of a compound provided herein, or a pharmaceutically acceptable salt thereof, in the treatment of an autoimmune disease.

In another aspect, provided herein is a compound of the present disclosure, or a pharmaceutically acceptable salt thereof, for use in the treatment of an autoimmune disease.

In an embodiment, the disease or disorder is a pemphigus disorder. In another embodiment, the pemphigus disorder is pemphigus vulgaris, pemphigus foliaceus, or bullous pemphigoid.

In yet another embodiment, the disease or disorder is diabetes. In still another embodiment, the diabetes is type 1 diabetes. In another embodiment, the diabetes is type 2 diabetes.

In an embodiment, the disease or disorder is selected from Sjogren's disease, systemic lupus erythematosus (SLE), arthritis, lupus nephritis, systemic sclerosis, multiple sclerosis (MS), autoimmune hepatitis, uveitis, myasthenia gravis, Hashimoto thyroiditis, thrombocytopenia purpura, myocarditis, atopic dermatitis, Goodpasture syndrome, hidradenitis suppurativa, ANCA vasculitis, dermatomyositis, primary biliary cirrhosis, polymyositis, temporal arteritis, alopecia areata, autoimmune hemolytic anemia, insulin resistance, a metabolic disease, fatty liver disease, hyperlipidemia, atherosclerosis, hypertension, stroke, gall bladder disease, and obesity.

In another embodiment, the disease or disorder is arthritis. In yet another embodiment, the arthritis is rheumatoid arthritis (RA) or psoriatic arthritis. In an embodiment, the arthritis is juvenile arthritis.

In still another embodiment, the disease or disorder is systemic lupus erythematosus (SLE).

In an embodiment, the disease or disorder is selected from hidradenitis suppurativa, a pemphigus disorder, dermatomyositis, and polymyositis. In another embodiment, the polymyositis is juvenile polymyositis.

In another embodiment, the disease or disorder is selected from insulin resistance, a metabolic disease, fatty liver disease, hyperlipidemia, atherosclerosis, hypertension, stroke, gall bladder disease, and obesity.

In another embodiment, the method or use relates to the treatment of lymphomas. In another embodiment, the lymphoma is Hodgkin's lymphoma, follicular lymphoma, Marginal Zone Lymphoma (MZL), Chronic Lymphocytic Leukemia (CLL), Small Lymphocytic Lymphoma (SLL), Mantle Cell Lymphoma (MCL), Diffuse Large B-Cell Lymphoma (DLBCL), or Lymphoplasmacytic Lymphoma.

In yet another aspect, provided herein is a method of treating a disease associated with the expression of high levels of CD74 in B cells in a subject, wherein the method comprises administering to the subject a therapeutically effective amount of a compound of the present disclosure, or a pharmaceutically acceptable salt thereof.

In an aspect, provided herein is a use of a compound provided herein, or a pharmaceutically acceptable salt thereof, in the manufacture of a medicament for the treatment of a disease associated with the expression of high levels of CD74 in B cells in a subject.

In another aspect, provided herein is a use of a compound provided herein, or a pharmaceutically acceptable salt thereof, in the treatment of a disease associated with the expression of high levels of CD74 in B cells in a subject.

In yet another aspect, provided herein is a compound of the present disclosure, or a pharmaceutically acceptable salt thereof, for use in the treatment of a disease associated with the expression of high levels of CD74 in B cells in a subject.

In an embodiment, the B-cell lymphoma is non-Hodgkin's lymphoma (NHL), Burkitt Lymphoma (BL) and multiple myeloma (MM).

In still another aspect, provided herein is a method of treating a B-cell lymphoma in a subject in need thereof, wherein the method comprises administering to the subject a therapeutically effective amount of a compound of the present disclosure, or a pharmaceutically acceptable salt thereof.

In another aspect, provided herein is a use of a compound provided herein, or a pharmaceutically acceptable salt thereof, in the manufacture of a medicament for the treatment of a B-cell lymphoma.

In yet another aspect, provided herein is a use of a compound provided herein, or a pharmaceutically acceptable salt thereof, in the treatment of a B-cell lymphoma.

In still another aspect, provided herein is a compound of the present disclosure, or a pharmaceutically acceptable salt thereof, for use in the treatment of a B-cell lymphoma.

In an embodiment, the B-cell lymphoma is non-Hodgkin's lymphoma (NHL), Burkitt Lymphoma (BL) and multiple myeloma (MM).

In an aspect, provided herein is a method for treating graft versus host disease (GvHD) in a subject after transplantation, wherein the method comprises administering to the subject a therapeutically effective amount of a compound of the present disclosure, or a pharmaceutically acceptable salt thereof, wherein the transplantation is the transplantation of a solid organ, a tissue or a cell population. In an embodiment, the treatment of graft versus host disease (GvHD) prevents rejection in clinical/surgical transplantation procedures of solid organs or cell populations.

In yet another aspect, provided herein is a method for preventing graft versus host disease (GvHD) in a subject after transplantation, wherein the method comprises administering to the subject prior to transplantation a therapeutically effective amount of a compound of the present disclosure, or a pharmaceutically acceptable salt thereof, wherein the transplantation is the transplantation of a solid organ, a tissue or a cell population.

In an aspect, provided herein is a use of a compound provided herein for treating graft versus host disease (GvHD) in a subject after transplantation, wherein the transplantation is the transplantation of a solid organ, a tissue or a cell population.

In another aspect, provided herein is a use of a compound provided herein in the manufacture of a medicament for treating graft versus host disease (GvHD) in a subject after transplantation, wherein the transplantation is the transplantation of a solid organ, a tissue or a cell population.

In yet another aspect, provided herein is a compound of the present disclosure for the use in treating graft versus host disease (GvHD) in a subject after transplantation, wherein the transplantation is the transplantation of a solid organ, a tissue or a cell population. In an embodiment, the transplantation is transplantation of a solid organ.

In another embodiment, the transplantation is bone marrow transplantation.

In yet another embodiment, the transplantation is stem cell transplantation.

In still another embodiment, the transplantation is hematopoietic stem cell transplantation.

In an embodiment, the transplantation is transplantation of a tissue.

In another embodiment, the graft versus host disease (GvHD) is an acute graft versus host disease.

In yet another embodiment, the graft versus host disease (GvHD) is a chronic graft versus host disease.

In an aspect, provided herein is a method of treating a condition selected from hidradenitis suppurativa, arthritis, a pemphigus disorder, ANCA vasculitis, diabetes, dermatomyositis, primary biliary cirrhosis, polymyositis, temporal arteritis, alopecia areata, autoimmune hemolytic anemia, insulin resistance, a metabolic disease, fatty liver disease, hyperlipidemia, atherosclerosis, hypertension, stroke, gall bladder disease, and obesity in a subject after transplantation, wherein the method comprises administering to the subject prior to transplantation a compound of provided herein, or a pharmaceutically acceptable salt thereof.

In an aspect, provided herein is a method of treating a condition selected from hidradenitis suppurativa, arthritis, a pemphigus disorder, ANCA vasculitis, diabetes, dermatomyositis, primary biliary cirrhosis, polymyositis, temporal arteritis, alopecia areata, autoimmune hemolytic anemia, insulin resistance, a metabolic disease, fatty liver disease, hyperlipidemia, atherosclerosis, hypertension, stroke, gall bladder disease, and obesity in a subject in need thereof comprising administering to the subject a compound of provided herein, or a pharmaceutically acceptable salt thereof.

In an embodiment, the compound is N²,4-dimethyl-N⁵—((R)-2-methyl-3-oxo-3-(((S)-11-oxo-2,3, 10,11-tetrahydro-1H,5H-benzo[d]pyrazolo[1,2-a][1,2]diazepin-10-yl-10-d)amino)propyl)thiazole-2,5-dicarboxamide, or a pharmaceutically acceptable salt thereof. In another embodiment, the compound is 4-methyl-N²-(methyl-d₃)—N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide, or a pharmaceutically acceptable salt thereof. In an embodiment, the pemphigus disorder is bullous pemphigoid.

In another embodiment, the diabetes is or type 2 diabetes.

In yet another embodiment, the polymyositis is juvenile polymyositis.

In an embodiment, the arthritis is juvenile arthritis.

In still another embodiment, the condition is selected from hidradenitis suppurativa, a pemphigus disorder, dermatomyositis, and polymyositis.

In another embodiment, the condition is selected from insulin resistance, a metabolic disease, fatty liver disease, hyperlipidemia, atherosclerosis, hypertension, stroke, gall bladder disease, and obesity.

In another aspect, provided herein is a method of treating a condition selected from hidradenitis suppurativa, arthritis, cutaneous lupus, a pemphigus disorder, ANCA vasculitis, diabetes, dermatomyositis, primary biliary cirrhosis, polymyositis, temporal arteritis, alopecia areata, autoimmune hemolytic anemia, insulin resistance, a metabolic disease, fatty liver disease, hyperlipidemia, atherosclerosis, hypertension, stroke, gall bladder disease, and obesity in a subject in need thereof, comprising administering to the subject a therapeutically effective amount of N²,4-dimethyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl)amino)propyl)thiazole-2,5-dicarboxamide, or a pharmaceutically acceptable salt thereof.

In an embodiment, the arthritis is juvenile arthritis or psoriatic arthritis. In another embodiment, the condition is cutaneous lupus.

In another embodiment, the pemphigus disorder is bullous pemphigoid.

In yet another embodiment, the diabetes is or type 2 diabetes.

In still another embodiment, the polymyositis is juvenile polymyositis.

In an embodiment, the condition is selected from hidradenitis suppurativa, a pemphigus disorder, dermatomyositis, and polymyositis. In another embodiment, the condition is selected from insulin resistance, a metabolic disease, fatty liver disease, hyperlipidemia, atherosclerosis, hypertension, stroke, gall bladder disease, and obesity.

In an aspect, provided herein is a method of treating a condition selected from psoriasis, autoimmune encephalomyelitis, autoimmune myositis, juvenile myositis, and gallstone disease in a subject in need thereof, comprising administering to the subject a therapeutically effective amount of N²,4-dimethyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl)amino)propyl)thiazole-2,5-dicarboxamide, or a pharmaceutically acceptable salt thereof.

In an aspect, provided herein is a method of treating a cancer selected from Marginal Zone Lymphoma (MZL), Chronic Lymphocytic Leukemia (CLL), Small Lymphocytic Lymphoma (SLL), Mantle Cell Lymphoma (MCL), Diffuse Large B-Cell Lymphoma (DLBCL), and Lymphoplasmacytic Lymphoma in a subject in need thereof, comprising administering to the subject a therapeutically effective amount of N²,4-dimethyl-N⁵—((R)-2-methyl-3-oxo-3-(((S)-11-oxo-2,3,10, 11-tetrahydro-1H,5H-benzo[d]pyrazolo[1,2-a][1,2]diazepin-10-yl)amino)propyl)thiazole-2,5-dicarboxamide, or a pharmaceutically acceptable salt thereof.

In an aspect, provided herein is a method of treating a condition selected from psoriasis, autoimmune encephalomyelitis, autoimmune myositis, juvenile myositis, and gallstone disease in a subject in need thereof, comprising administering to the subject a therapeutically effective amount of N²,4-dimethyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide, or a pharmaceutically acceptable salt thereof.

In an aspect, provided herein is a method of treating a cancer selected from Marginal Zone Lymphoma (MZL), Chronic Lymphocytic Leukemia (CLL), Small Lymphocytic Lymphoma (SLL), Mantle Cell Lymphoma (MCL), Diffuse Large B-Cell Lymphoma (DLBCL), and Lymphoplasmacytic Lymphoma in a subject in need thereof, comprising administering to the subject a therapeutically effective amount of N²,4-dimethyl-N⁵—((R)-2-methyl-3-oxo-3-(((S)-11-oxo-2,3,10, 11-tetrahydro-1H,5H-benzo[d]pyrazolo[1,2-a][1,2]diazepin-10-yl-10-d)amino)propyl)thiazole-2,5-dicarboxamide, or a pharmaceutically acceptable salt thereof.

In an aspect, provided herein is a method of treating a condition selected from psoriasis, autoimmune encephalomyelitis, autoimmune myositis, juvenile myositis, and gallstone disease in a subject in need thereof, comprising administering to the subject a therapeutically effective amount of 4-methyl-N²-(methyl-d₃)—N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide, or a pharmaceutically acceptable salt thereof.

In an aspect, provided herein is a method of treating a cancer selected from Marginal Zone Lymphoma (MZL), Chronic Lymphocytic Leukemia (CLL), Small Lymphocytic Lymphoma (SLL), Mantle Cell Lymphoma (MCL), Diffuse Large B-Cell Lymphoma (DLBCL), and Lymphoplasmacytic Lymphoma in a subject in need thereof, comprising administering to the subject a therapeutically effective amount of 4-methyl-N²-(methyl-d₃)—N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide, or a pharmaceutically acceptable salt thereof.

### Combination Therapy

In certain instances, it may be advantageous to administer a compound of the present disclosure in combination with one or more additional therapeutic agents. A therapeutic agent is, for example, a chemical compound, peptide, antibody, antibody fragment or nucleic acid, which is therapeutically active or enhances the therapeutic activity when administered to a patient in combination with a compound of the present disclosure.

Compounds of the disclosure may be administered as the sole active ingredient or together with other drugs useful against neoplastic diseases, inflammatory disorders, in immunomodulating regimens or in induction therapy to prevent GvHD and transplant rejection. For example, the compounds of the disclosure may be used in combination e.g., with cyclosporins, rapamycins or ascomycins, or their immunosuppressive analogs or derivatives, e.g., cyclosporin A, cyclosporin G, Isa tx247, FK-506, sirolimus or everolimus; with corticosteroids e.g., prednisone; cyclophosphamide; azathioprene; methotrexate; gold salts; sulfasalazine, antimalarials; leflunomide; mizoribine; mycophenolic acid; mycophenolate mofetil; 15-deoxyspergualine; with a SIP receptor agonist e.g., FTY720 or an analogue thereof; with immuno-suppressive monoclonal antibodies, e.g., monoclonal antibodies to leukocyte receptors, e.g., MHC, or other immunomodulatory compounds, e.g., CTLA4 g.

A compound of formula I may also be used in combination with other antiproliferative agents. Such antiproliferative agents include, but are not limited to aromatase inhibitors, antiestrogens, topoisomerase I inhibitors, topoisomerase II inhibitors, microtubule active agents, alkylating agents, histone deacetylase inhibitors, farnesyl transferase inhibitors, COX-2 inhibitors, MMP inhibitors, mTOR inhibitors, antineoplastic antimetabolites, platin compounds, compounds decreasing the protein kinase activity and further anti-angiogenic compounds, gonadorelin agonists, anti-androgens, bengamides, bisphosphonates, antiproliferative antibodies and temozolomide (TEMODAL).

### Examples

The compounds of the present disclosure can be produced as shown in the following examples. The following examples are intended to illustrate the disclosure and are not to be construed as being limitations thereon. Temperatures are given in degrees Celsius. If not mentioned otherwise, all evaporations are performed under reduced pressure, typically between about 15 mm Hg and 100 mm Hg (═20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present disclosure are either commercially available or can be produced by organic synthesis methods known to one of ordinary skill in the art or can be produced by organic synthesis methods as described herein.

For illustrative purposes, the general reaction schemes depicted herein provide potential routes for synthesizing the compounds of the present disclosure as well as key intermediates. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art.

### Abbreviations

ACN acetonitrile
d doublet
DIPEA N,N-diisopropylethylamine
DMSO dimethylsulfoxide
EDC 1-ethyl-3-(3-dimethylaminopropyl)carbodiimid
Et₂O diethylether
Flow flow rate
h hour(s)
HCl hydrochloric acid
HPLC High Performance Liquid Chromatography
LCMS Liquid Chromatography/Mass Spectrometry
M molar (mol/L)
min minute(s)
mL milliliter
mm millimeter
MHz megaHertz
MS
MTBE
Mass Spectrometry
methyl tert-butyl ether
NMR Nuclear Magnetic Resonance
q Quartet
rt room temperature
t_{R} retention time
S singlet
sat. saturated
t triplet
TFA trifluoroacetic acid
TOTU O-[(Ethoxycarbonyl) cyanomethylenamino]-N,N,N, N-tetramethyluronium tetrafluoroborate

### Analytical Methods

### HPLC Conditions:

Method a: HPLC Instrument: Agilent 1100 series; Column: Waters X-Bridge C18 2.5 μm 3*30 mm, Eluent A: water+0.1% TFA, B: ACN+0.1% TFA, Gradient 10 to 98% B in 3 min, Flow: 1.4 mL/min Method b: HPLC Instrument: Agilent 1100 series; Column: Waters X-Bridge C18 2.5 μm 3*50 mm, Eluent A: water+0.1% TFA, B: ACN+0.1% TFA, Gradient 10 to 98% B in 8.6 min, Flow: 1.4 mL/min

### Synthesis of Example Compounds

The syntheses of int-A1 and Example A, as well as the activity of Example A against SPPL2a, have been previously disclosed in International Application No. PCT/IB2021/058398 (WO 2022/058902), which is incorporated by reference in its entirety.

### Synthesis of(S)-10-amino-2,3,5,10-tetrahydro-1H,11H-benzo[d]pyrazolo[1,2-a][1,2]diazepin-11-one (int-A1)

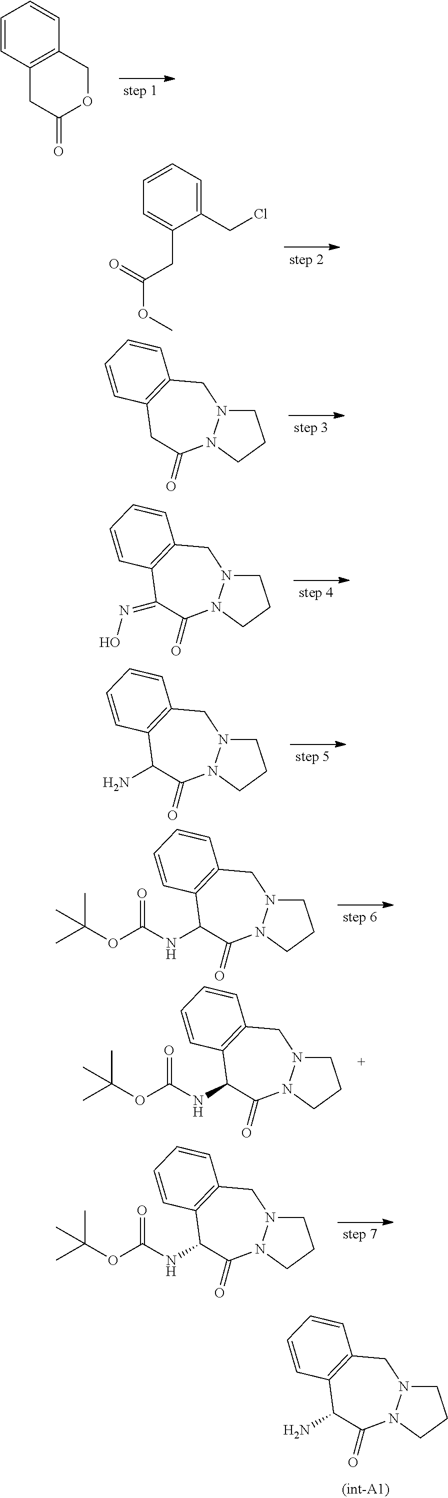

Step 1: Thionyl chloride (14.8 mL, 202 mmol) was added dropwise at 0° C. to a suspension of isochroman-3-one (15 g, 101 mmol) in methanol (150 mL). The resulting solution was stirred at 0° C. for 2 h followed by stirring at rt for 16 h. The reaction mixture was concentrated, the crude material was dissolved in Ethyl acetate and washed with sat. aq. NaHCO₃ solution. The organic layer was dried (Na₂SO₄) and concentrated to give methyl 2-(2-(chloromethyl)phenyl) acetate. ¹H NMR (DMSO-d₆, 400 MHZ): δ 7.44-7.47 (m, 1H), 7.27-7.36 (m, 3H), 4.80 (s, 2H), 3.85 (s, 2H), 3.63 (s, 3H).

Step 2: Pyrazolidine dihydrochloride (14.2 g, 98 mmol) was added at rt to a solution of methyl 2-(2-(chloromethyl)phenyl) acetate (19.4 g, 98 mmol) in DMF (500 mL) followed by DIPEA (85 mL, 488 mmol), sodium iodide (14.6 g, 98 mmol) and sodium acetate (32.0 g, 391 mmol). The suspension was stirred at rt for 16 h. The reaction mixture was concentrated, the crude material was dissolved in ethyl acetate and washed with sat. NaHCO₃ solution. The organic phase was dried (Na₂SO₄), concentrated and purified by column chromatography (10-20% ethyl acetate in toluene) to give 2,3,5,10-tetrahydrobenzo[d]pyrazolo[1,2-a][1,2]diazepin-11 (1H)-one. LCMS (method d) m/z 203.1 [M+H]⁺, t_{R}=1.35 min. ¹H NMR (DMSO-d₆, 400 MHZ): δ 7.14-7.26 (m, 3H), 7.03 (d, J=7.3 Hz, 1H), 4.15 (s, 2H), 3.84 (br s, 2H), 3.48 (t, J=7.2 Hz, 2H), 3.19 (t, J=6.7 Hz, 2H), 2.19 (quin, J=7.0 Hz, 2H).

Step 3: 1M LiHMDS solution in THF (93 mL, 93 mmol) was added dropwise at 0° C. to a solution of 2,3,5, 10-tetrahydrobenzo[d]pyrazolo[1,2-a][1,2]diazepin-11 (1H)-one (14.0 g, 62 mmol) and isopentyl nitrite (10.8 mL, 81 mmol) in THF (750 mL), and the solution was stirred at 0° C. for 2 h. The reaction mixture was concentrated and dissolved in ethyl acetate, washed with sat. NaHCO₃ solution, dried (Na₂SO₄), concentrated and purified by column chromatography (10-90% ethyl acetate in toluene with 0.1% Et3N) to provide a mixture of (Z) and (E)-10-(hydroxyimino)-2,3,5, 10-tetrahydrobenzo[d]pyrazolo[1,2-a][1,2]diazepin-11 (1H)-one. LCMS (method d) m/z 232.0 [M+H]⁺, t_{R}=0.90 & 1.06 min.

Step 4: Zinc dust (10.9 g, 166 mmol) was added at rt to a solution of (Z) and (E)-10-(hydroxyimino)-2,3,5, 10-tetrahydrobenzo[d]pyrazolo[1,2-a][1,2]diazepin-11 (1H)-one (9.6 g, 42 mmol) in AcOH (300 mL) and a 10% HCl aq. solution (300 mL) and the reaction mixture was stirred at rt for 2 h. The reaction mixture was filtered and the filtrate was concentrated under vacuum to give 10-amino-2,3,5, 10-tetrahydrobenzo[d]pyrazolo[1,2-a][1,2]diazepin-11 (1H)-one which was used in the next step without further purification.

Step 5: BoczO (9.0 g, 41 mmol) and Na₂CO₃ (13.0 g, 124 mmol) were added at rt to a solution of 10-amino-2,3,5, 10-tetrahydrobenzo[d]pyrazolo[1,2-a][1,2]diazepin-11 (1H)-one (29.2 g, 41 mmol) in dioxane (400 mL) and water (200 mL), and the resulting mixture was stirred at rt for 16 h. The mixture was concentrated and treated with ethyl acetate and sat. NaHCO₃ solution. The organic layer was dried (MgSO₄) and concentrated to give the crude product which was purified by column chromatography (0-80% ethyl acetate in cyclohexane) to yield racemic tert-butyl 11-oxo-1,2,3,5,10,11-hexahydrobenzo[d]pyrazolo[1,2-a][1,2]diazepin-10-ylcarbamate.

Step 6 (chiral separation); The two enantiomers of tert-butyl 11-oxo-1,2,3,5,10,11-hexahydrobenzo[d]pyrazolo[1,2-a][1,2]diazepin-10-ylcarbamate were separated by chiral HPLC (Thar SFC-200 instrument, mobile phase: scCO2/EtOH 85:15, column: Chiralcel OD-H, 30×250 mm) to provide tert-butyl(S)-(11-oxo-2,3,10,11-tetrahydro-1H,5H-benzo[d]pyrazolo[1,2-a][1,2]diazepin-10-yl)carbamate (enantiomeric excess ≥99.5%) and tert-butyl (R)-(11-oxo-2,3,10,11-tetrahydro-1H,5H-benzo[d]pyrazolo[1,2-a][1,2]diazepin-10-yl)carbamate (enantiomeric excess ≥99.5%). Analytical data for tert-butyl(S)-(11-oxo-2,3,10,11-tetrahydro-1H,5H-benzo[d]pyrazolo[1,2-a][1,2]diazepin-10-yl)carbamate: LCMS (method b) m/z 318.3 [M+H]⁺, t_{R}=1.03 min. ¹H NMR (400 MHZ, DMSO-d₆) δ ppm 7.31 (d, J=7.3 Hz, 1H), 7.15-7.27 (m, 2H), 7.05 (d, J=6.7 Hz, 1H), 7.00 (d, J=9.1 Hz, 1H), 6.42 (d, J=9.1 Hz, 1H), 4.22 (s, 2H), 3.42-3.60 (m, 2H), 3.22-3.30 (m, 1H), 3.13-3.21 (m, 1H), 2.27-2.41 (m, 1H), 2.04-2.16 (m, 1H), 1.43 (s, 9H).

Step 7: tert-butyl(S)-(11-oxo-2,3,10,11-tetrahydro-1H,5H-benzo[d]pyrazolo[1,2-a][1,2]diazepin-10-yl)carbamate (17.7 g, 56 mmol) was treated with 4M HCl in dioxane (250 mL) and the mixture was stirred at rt for 1 h. The reaction mixture was concentrated to yield(S)-10-amino-2,3,5,10-tetrahydro-1H,11H-benzo[d]pyrazolo[1,2-a][1,2]diazepin-11-one (int-A1) as an HCl salt. LCMS (method b) m/z 218.3 [M+H]⁺, t_{R}=0.40 min. Stereochemistry confirmed by X-ray analysis: [α]²³D-105.7 (c=1.0, MeOH). ¹H NMR (400 MHZ, DMSO-d₆) δ ppm 8.89 (br s, 3H), 7.29-7.39 (m, 2H), 7.24 (d, J=7.6 Hz, 1H), 7.16 (d, J=7.1 Hz, 1H), 5.98 (s, 1H), 4.28 (s, 2H), 3.55-3.62 (m, 2H), 3.18-3.29 (m, 2H), 2.29-2.44 (m, 1H), 2.12-2.19 (m, 1H).

### Synthesis of 2-(ethylcarbamoyl)-4-methylthiazole-5-carboxylic acid (int-EC2)

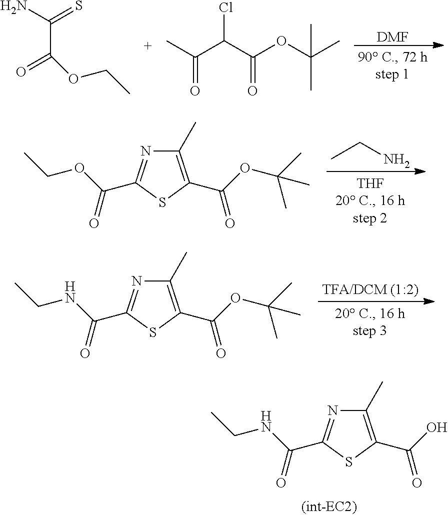

Step 1: Ethyl 2-amino-2-thioxoacetate (1.7 g. 12.8 mmol) was added to a solution of tert-butyl 2-chloro-3-oxobutanoate (2.46 g. 12.8 mmol) in DMF (10 mL), and the solution stirred 3 days at 90° C. After cooling to rt, the reaction mixture was concentrated and the residue purified by column chromatography (0-50% ethyl acetate in cyclohexane) to give S-(tert-butyl) 2-ethyl 4-methylthiazole-2,5-dicarboxylate. LCMS (method e) m/z 272.1 [M+H]⁺, t_{R}=1.23 min. ¹H NMR (400 MHZ, DMSO-d₆) δ ppm 4.38 (d, J=7.1 Hz, 2H), 2.67 (s, 3H), 1.54 (s, 9H), 1.33 (t, J=7.0 Hz, 3H).

Step 2: A solution of 5-(tert-butyl) 2-ethyl 4-methylthiazole-2,5-dicarboxylate (500 mg, 1.84 mmol) in 2M solution of ethanamine in THF (27.6 mL. 55.2 mmol) was stirred at room temperature for 16 h. The reaction mixture was treated with ethyl acetate and washed with sat. NaHCO₃ and 1N HCl. The organic phase was dried (MgSO₄) and concentrated to give tert-butyl 2-(ethylcarbamoyl)-4-methylthiazole-5-carboxylate. LCMS (method b) m/z 271.1 [M+H]⁺, t_{R}=1.14 min. ¹H NMR (400 MHZ, DMSO-d₆) δ ppm 8.97 (t, J=6.1 Hz, 1H), 3.30-3.23 (m, 2H), 2.66 (s, 3H), 1.53 (s, 9H), 1.11 (t, J=7.3 Hz, 3H).

Step 3: A solution of tert-butyl 2-(ethylcarbamoyl)-4-methylthiazole-5-carboxylate (540 mg, 1.8 mmol) in TFA (15 mL) and CH₂Cl₂ (30 mL) was stirred at rt for 16 h. The reaction mixture was concentrated and treated with Et₂O. The precipitate was filtered off, washed with cold Et2O and dried in vacuum to give 2-(ethylcarbamoyl)-4-methylthiazole-5-carboxylic acid (int-EC2). LCMS (method b) m/z 215.1 [M+H]⁺, t_{R}=0.54 min. ¹H NMR (400 MHZ, DMSO-d₆) δ ppm 8.97 (t, J=6.0 Hz, 1H), 3.30-3.26 (m, 2H), 2.67 (s, 3H), 1.12 (q, J=7.4 Hz, 3H)(acid proton not seen).

### Synthesis of 4-methyl-2-(methylcarbamoyl)thiazole-5-carboxylic acid (int-EC4)

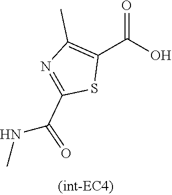

4-Methyl-2-(methylcarbamoyl)thiazole-5-carboxylic acid (int-EC4) was obtained using an analogous method as that described for the synthesis of 2-(ethylcarbamoyl)-4-methylthiazole-5-carboxylic acid (int-EC2), except in step 2 where ethanamine in THF was replaced with methamine in ethanol. LCMS (method a) m/z 201.1 M+H]⁺, t_{R}=0.45 min.

### Int-IN1. 2-Carbamoyl-4-methylthiazole-5-carboxylic acid

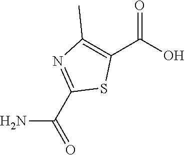

This compound was prepared according to the procedure described in int-EC2, with ammonia (7 N in MeOH, Sigma-Aldrich, 499145) replacing ethanamine in step 2. Retention time on LC-MS tᵣ=0.543 min, LC-MS calculated for C₆H₇N₂O₃S (M+H)⁺: m/z=187.0; found 187.1.

### Int-IN2. 4-Methyl-2-((methyl-d₃)carbamoyl)thiazole-5-carboxylic acid

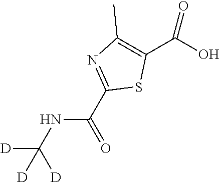 [SMILES: [2H]C([2H])([2H])NC(=O)c1nc(C)c(C(=O)O)s1]

This compound was prepared according to the procedure described in int-EC2, with a 1:2 molar ratio of methyl-d₃-amine hydrochloride (Cambridge Isotope, DLM-289-1) and potassium tert-butoxide replacing ethanamine in step 2. Retention time on LC-MS tᵣ=0.668 min, LC-MS calculated for C₇H₆D₃N₂O₃S (M+H)⁺: m/z=204.1; found 204.2.

### Example A: N²,4-dimethyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl)amino)propyl)thiazole-2,5-dicarboxamide (A)

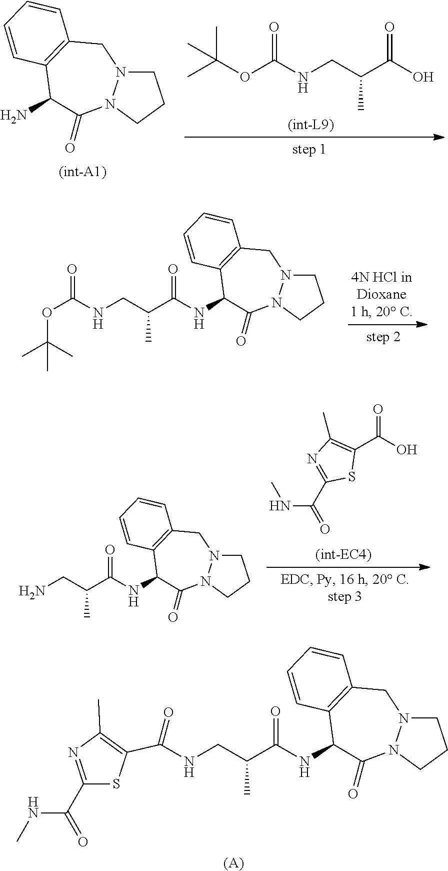

Step 1: To a solution of(S)-10-amino-2,3,5, 10-tetrahydro-1H, 11H-benzo[d]pyrazolo[1,2-a][1,2]diazepin-11-one (int-A1)(5.0 g, 11.12 mmol) in CH₃CN (50 mL), were added (R)-3-((tert-butoxycarbonyl)amino)-2-methylpropanoic acid (int-L9)(2.26 g, 11.12 mmol), DIPEA (5.83 mL, 33.4 mmol) and TOTU (3.65 g, 11.12 mmol) and the reaction mixture was stirred at rt for 16 h. The reaction mixture was then concentrated and the crude product was dissolved in ethyl acetate and washed with 1N HCl and sat. NaHCO₃, dried (MgSO₄) and concentrated to provide an oil which was treated with cold Et2O to crystalize the product. The obtained precipitate was collected by filtration and dried in vacuum to give tert-butyl ((R)-2-methyl-3-oxo-3-(((S)-11-oxo-2,3,10,11-tetrahydro-1H,5H-benzo[d]pyrazolo[1,2-a][1,2]diazepin-10-yl)amino)propyl)carbamate. LCMS (method a) m/z 403.1 [M+H]⁺, t_{R}=0.92 min. ¹H NMR (400 MHz, DMSO-d₆) δ ppm: 8.21 (d, J=8.8 Hz, 1H), 7.29 (d, J=7.9 Hz, 1H), 7.23 (t, J=7.4 Hz, 1H), 7.15 (t, J=7.6 Hz, 1H), 7.06 (d, J=7.6 Hz, 1H), 6.80 (d, J=4.0 Hz, 1H), 6.70 (d, J=8.7 Hz, 1H), 4.24 (s, 2H), 3.62-3.43 (m, 2H), 3.31-3.22 (m, 1H), 3.21-3.08 (m, 2H), 3.00-2.91 (m, 1H), 2.89-2.79 (m, 1H), 2.42-2.27 (m, 1H), 2.16-2.07 (m, 1H), 1.40 (s, 9H), 1.01 (d, J=6.7 H₂, 3H).

Step 2: tert-Butyl ((R)-2-methyl-3-oxo-3-(((S)-11-oxo-2,3,10,11-tetrahydro-1H,5H-benzo[d]pyrazolo[1,2-a][1,2]diazepin-10-yl)amino)propyl)carbamate (4.92 g, 11.0 mmol) was dissolved in 4N HCl (50 mL) and the reaction mixture was stirred at rt for 1 h. The reaction mixture was concentrated and then CH₃CN/Et₂O was added. The resulting precipitate was filtered off, washed with cold Et2O and dried under vaccum to give (R)-3-amino-2-methyl-N—((S)-11-oxo-2,3,10,11-tetrahydro-1H,5H-benzo[d]pyrazolo[1,2-a][1,2]diazepin-10-yl)propanamide. LCMS (method a) m/z 303 [M+H]⁺, t_{R}=0.41 min. ¹H NMR (400 MHZ, DMSO-d₆) δ ppm: 8.74 (d, J=9.2 Hz, 1H), 7.79 (s, 3H), 7.31 (d, J=7.7 Hz, 1H), 7.24 (t, J=7.0 Hz, 1H), 7.18 (t, J=7.0 Hz, 1H), 7.07 (d, J=7.5 Hz, 1H), 6.77 (d, J=9.1 Hz, 1H), 4.25 (s, 2H), 3.56-3.49 (m, 2H), 3.33-3.24 (m, 1H), 3.22-3.14 (m, 1H), 3.11-2.98 (m, 2H), 2.94-2.79 (m, 1H), 2.43-2.30 (m, 1H), 2.18-2.03 (m, 1H), 1.20 (d, J=6.5 Hz, 3H).

Step 3:4-Methyl-2-(methylcarbamoyl)thiazole-5-carboxylic acid (int-EC4)(10 mg, 0.04 mmol) and EDC (14.71 mg, 0.077 mmol) were added to a solution of (R)-3-amino-2-methyl-N—((S)-11-oxo-2,3,10,11-tetrahydro-1H,5H-benzo[d]pyrazolo[1,2-a][1,2]diazepin-10-yl)propanamide (13 mg, 0.038 mmol) in pyridine (2 mL) and the mixture was stirred at rt for 16 h. Ethyl acetate was added to the reaction mixture and the organic phase was washed with sat. NaHCO₃, dried (MgSO₄) and concentrated. The crude product was purified by preparative HPLC (Waters SunFire Prep C18 OBD 5 μm, 30*100 mm, Flow: 40 mL/min, CH₃CN: 5 min to 5%, 25 min to 60%) to yield N²,4-dimethyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl)amino)propyl)thiazole-2,5-dicarboxamide (1). LCMS (method b) m/z 485.2 [M+H]⁺, t_{R}=0.73 min. ¹H NMR (400 MHZ, DMSO-d₆) δ ppm: 8.84 (q, J=4.0 Hz, 1H), 8.40 (t, J=5.6 Hz, 1H), 8.36 (d, J=8.9 Hz, 1H), 7.23 (d, J=7.9 Hz, 1H), 7.19 (t, J=7.5 Hz, 1H), 7.05 (d, J=7.6 Hz, 1H), 6.92 (t, J=7.6 Hz, 1H), 6.73 (d, J=8.8 Hz, 1H), 4.24 (s, 2H), 3.63-3.40 (m, 3H), 3.29-3.24 (m, 1H), 3.23-3.14 (m, 1H), 3.13-3.01 (m, 1H), 2.80 (d, J=4.8 Hz, 3H), 2.59 (s, 3H), 2.43-2.26 (m, 2H), 2.17-2.03 (m, 1H), 1.10 (d, J=6.9 Hz, 3H).

Alternatively, the resulting product was triturated in MTBE (or acetonitrile) and the suspension was filtered to obtain the solid. The solid was then dried under vacuum to afford compound of example A in a crystalline form.

### Examples 1 and 2. N²,4-Dimethyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide (1) and N²,4-dimethyl-N⁵—((R)-2-methyl-3-oxo-3-(((R)-11-oxo-2,3,10,11-tetrahydro-1H,5H-benzo[d]pyrazolo[1,2-a][1,2]diazepin-10-yl-10-d)amino)propyl)thiazole-2,5-dicarboxamide (2)

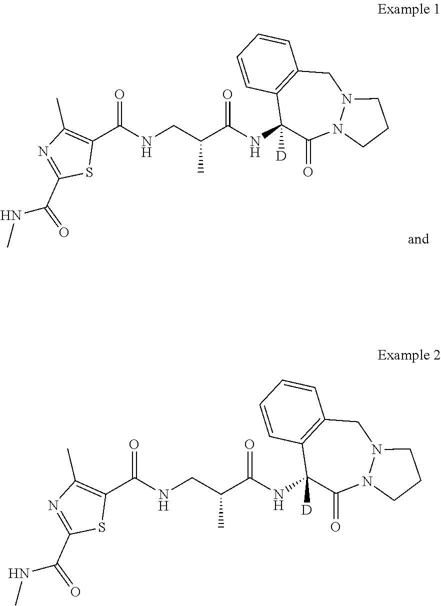 [SMILES: [2H][C@@]1(NC(=O)[C@H](C)CNC(=O)c2sc(C(=O)NC)nc2C)C(=O)N2CCCN2Cc2ccccc21 ; [2H][C@]1(NC(=O)[C@H](C)CNC(=O)c2sc(C(=O)NC)nc2C)C(=O)N2CCCN2Cc2ccccc21]

To a mixture of N²,4-dimethyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl)amino)propyl)thiazole-2,5-dicarboxamide (Example A)(100.0 mg, 0.206 mmol) in methanol-^{d4} (5 mL, Cambridge Isotope Laboratories DLM-24-10) was added potassium tert-butoxide (20.0 mg, 0.179 mmol), and the mixture was stirred at 60° C. for 1 h. After cooling to rt, the reaction mixture was quenched with a 1% v/v solution of acetic acid-d₁ (Sigma-Aldrich 151777) in acetonitrile (5 mL). After stirring for 5 minutes, the reaction mixture was further diluted with water, and the diastereomeric mixture was filtered and purified by prep-HPLC (Sunfire C18 column, eluting with a gradient of acetonitrile/water containing 0.1% TFA, at flow rate of 60 mL/min) to afford each diastereomer as its TFA salt.

Example 1: Retention time on LC-MS tᵣ=1.76 min, LC-MS calculated for C₂₃H₂₈DN₆O₄S (M+H)⁺: m/z=486.2; found 486.2. 1H NMR (500 MHz, DMSO-d₆) δ 8.84 (q, J=4.7 Hz, 1H), 8.40 (t, J=5.5 Hz, 1H), 8.36 (s, 1H), 7.23 (d, J=7.9 Hz, 1H), 7.18 (t, J=7.4 Hz, 1H), 7.05 (d, J=7.6 Hz, 1H), 6.92 (t, J=7.6 Hz, 1H), 4.24 (s, 2H), 3.60-3.43 (m, 3H), 3.33-3.24 (m, 2H), 3.22-3.14 (m, 1H), 3.12-3.04 (m, 1H), 2.80 (d, J=4.7 Hz, 3H), 2.59 (s, 3H), 2.41-2.30 (m, 1H), 2.18-2.04 (m, 1H), 1.10 (d, J=6.9 Hz, 3H).

Example 2: Retention time on LC-MS tᵣ=1.84 min, LC-MS calculated for C₂₃H₂₈DN₆O₄S (M+H)⁺: m/z=486.2; found 486.2. 1H NMR (500 MHZ, DMSO-d₆) δ 8.81 (q, J=4.7 Hz, 1H), 8.44 (t, J=5.7 Hz, 1H), 8.40 (s, 1H), 7.28-7.15 (m, 3H), 7.10-7.04 (m, 1H), 4.25 (s, 2H), 3.55-3.47 (m, 2H), 3.42-3.23 (m, 3H), 3.22-3.14 (m, 1H), 3.12-3.01 (m, 1H), 2.79 (d, J=4.7 Hz, 3H), 2.57 (s, 3H), 2.42-2.29 (m, 1H), 2.15-2.06 (m, 1H), 1.16 (d, J=6.8 Hz, 3H).

### Examples 3 and 4. N²,4-Dimethyl-N⁵—((R)-2-methyl-3-(((S)-10-methyl-11-oxo-2,3,10,11-tetrahydro-1H,5H-benzo[d]pyrazolo[1,2-a][1,2]diazepin-10-yl)amino)-3-oxopropyl)thiazole-2,5-dicarboxamide and N²,4-dimethyl-N⁵—((R)-2-methyl-3-(((R)-10-methyl-11-oxo-2,3,10,11-tetrahydro-1H,5H-benzo[d]pyrazolo[1,2-a][1,2]diazepin-10-yl)amino)-3-oxopropyl)thiazole-2,5-dicarboxamide

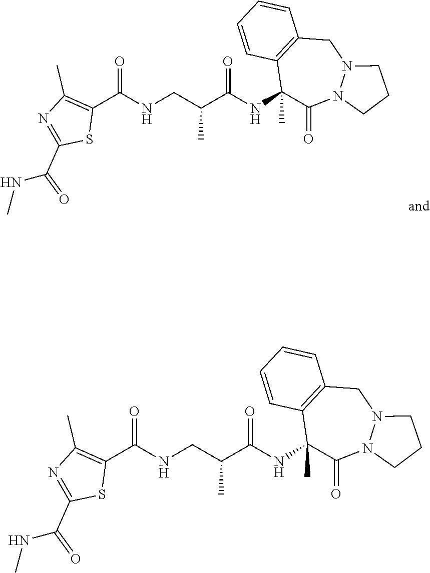 [SMILES: CNC(=O)c1nc(C)c(C(=O)NC[C@@H](C)C(=O)N[C@]2(C)C(=O)N3CCCN3Cc3ccccc32)s1 ; CNC(=O)c1nc(C)c(C(=O)NC[C@@H](C)C(=O)N[C@@]2(C)C(=O)N3CCCN3Cc3ccccc32)s1]

### Step 1:2-(11-Oxo-2,3,10,11-tetrahydro-1H,5H-benzo[d]pyrazolo[1,2-a][1,2]diazepin-10-yl)isoindoline-1,3-dione

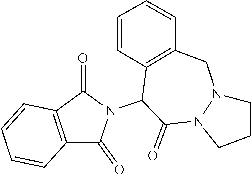 [SMILES: O=C1c2ccccc2C(=O)N1C1C(=O)N2CCCN2Cc2ccccc21]

To a mixture of 10-amino-2,3,5,10-tetrahydro-1H,11H-benzo[d]pyrazolo[1,2-a][1,2]diazepin-11-one (int-A1, step 4)(993 mg, 4.57 mmol) and triethylamine (2.31 g, 22.9 mmol) in CH₂Cl₂ (40 mL) was added phthaloyl dichloride (1.02 g, 5.03 mmol) and the mixture was stirred at rt for 30 min. The mixture was diluted with CH₂Cl₂ (40 mL) and washed with water (50 mL) and saturated aqueous NaCl. The organic layer was dried over Na₂SO₄, concentrated and the crude residue was purified by flash chromatography (SiO₂, EtOAc/hexanes) to afford the desired product (610 mg, 38% yield) as a yellow solid. LC-MS calculated for C₂₀H₁₈N₃O₃ (M+H)⁺: m/z=348.1; found 348.2.

### Step 2:2-(10-Methyl-11-oxo-2,3,10,11-tetrahydro-1H,5H-benzo[d]pyrazolo[1,2-a][1,2]diazepin-10-yl)isoindoline-1,3-dione

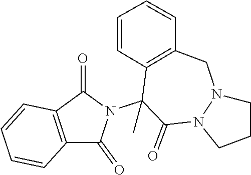 [SMILES: CC1(N2C(=O)c3ccccc3C2=O)C(=O)N2CCCN2Cc2ccccc21]

In an oven-dried vial with a stir bar, to a mixture of 2-(11-oxo-2,3,10,11-tetrahydro-1H,5H-benzo[d]pyrazolo[1,2-a][1,2]diazepin-10-yl)isoindoline-1,3-dione (487 mg, 1.40 mmol) in DMF (14 mL) was added sodium hydride (112 mg, 2.80 mmol)(60% dispersion in mineral oil, Aldrich 452912) and the mixture was stirred at rt for 15 min before iodomethane (398 mg, 2.80 mmol) was added and the reaction mixture was stirred at rt for 1 hour. The reaction mixture was diluted with ethyl acetate (100 mL), washed with water (100 mL) and saturated aqueous NaCl. The organic layer was dried over Na₂SO₄, concentrated and the crude residue was purified by flash chromatography (SiO₂, EtOAc/hexanes) to afford the desired product (313 mg, 62% yield) as an off-white solid. LC-MS calculated for C₂₁H₂₀N₃O₃ (M+H)⁺: m/z=362.2; found 362.2.

### Step 3:10-Amino-10-methyl-2,3,5,10-tetrahydro-1H, 11H-benzo[d]pyrazolo[1,2-a][1,2]diazepin-11-one

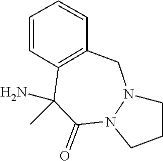 [SMILES: CC1(N)C(=O)N2CCCN2Cc2ccccc21]

To a mixture of 2-(10-methyl-11-oxo-2,3,10,11-tetrahydro-1H,5H-benzo[d]pyrazolo[1,2-a][1,2]diazepin-10-yl)isoindoline-1,3-dione (313 mg, 0.866 mmol) in MeOH (15 mL) was added a 1 molar solution of hydrazine in THF (4.5 mL, 4.5 mmol, Aldrich 433632) and the reaction mixture was irradiated at 120° C. in a microwave reactor for 6 hours. After cooling to rt the reaction mixture was concentrated in vacuo. The crude residue was diluted with CH₂Cl₂ and 1 N NaOH and the layers were separated. The organic layer was removed, and the aqueous layer was extracted with CH₂Cl₂. The combined organic layers were washed with brine, dried over Na₂SO₄, and concentrated to give the desired compound (182 mg, 91% yield) as a colorless oil. LC-MS calculated for C₁₃H₁₈N₃O (M+H)⁺: m/z=232.1; found 232.2.

### Step 4: tert-Butyl ((2R)-2-methyl-3-((10-methyl-11-oxo-2,3,10,11-tetrahydro-1H,5H-benzo[d]pyrazolo[1,2-a][1,2]diazepin-10-yl)amino)-3-oxopropyl)carbamate

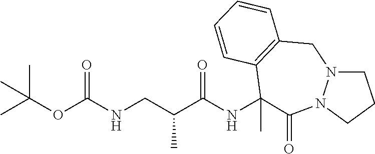 [SMILES: C[C@H](CNC(=O)OC(C)(C)C)C(=O)NC1(C)C(=O)N2CCCN2Cc2ccccc21]

To a mixture of 10-amino-10-methyl-2,3,5, 10-tetrahydro-1H,11H-benzo[d]pyrazolo[1,2-a][1,2]diazepin-11-one (182 mg, 0.790 mmol) and (R)-3-((tert-butoxycarbonyl)amino)-2-methylpropanoic acid (192 mg, 0.947 mmol, Combi-Blocks QB-0994) in CH₂Cl₂ (7 mL) and pyridine (2.5 mL) was added EDC (454 mg, 2.37 mmol) and the reaction mixture was stirred at 40° C. for 3 h. After cooling to rt, the reaction mixture was concentrated in vacuo. The crude residue was purified by flash chromatography (SiO₂, EtOAc/hexanes) to afford the title compound (281 mg, 86% yield) as a mixture of diastereomers in the form of a colorless oil. LC-MS calculated for C₂₂H₃₃N₄O₄ (M+H)⁺: m/z=417.3; found 417.3.

### Step 5: (2R)-3-Amino-2-methyl-N-(10-methyl-11-oxo-2,3,10,11-tetrahydro-1H,5H-benzo[d]pyrazolo[1,2-a][1,2]diazepin-10-yl)propanamide

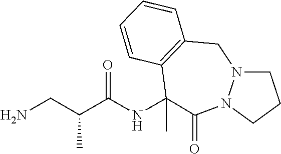 [SMILES: C[C@H](CN)C(=O)NC1(C)C(=O)N2CCCN2Cc2ccccc21]

To a mixture of tert-butyl ((2R)-2-methyl-3-((10-methyl-11-oxo-2,3,10,11-tetrahydro-1H,5H-benzo[d]pyrazolo[1,2-a][1,2]diazepin-10-yl)amino)-3-oxopropyl)carbamate (281 mg, 0.676 mmol) in CH₂Cl₂ (10 mL) was added a 4 molar solution of HCl in dioxane (3 mL, 12 mmol, Sigma-Aldrich 345547) and the reaction mixture was stirred at rt for 2 h. The reaction mixture was concentrated to give a crude colorless oil containing the desired product as a mixture of diastereomers (as HCl salt), which was used without further purification. LC-MS calculated for C₁₇H₂₅N₄O₂ (M+H)⁺: m/z=317.2; found 317.3.

### Step 6: N²,4-Dimethyl-N⁵—((R)-2-methyl-3-(((S)-10-methyl-11-oxo-2,3, 10,11-tetrahydro-1H,5H-benzo[d]pyrazolo[1,2-a][1,2]diazepin-10-yl)amino)-3-oxopropyl)thiazole-2,5-dicarboxamide and N²,4-dimethyl-N⁵—((R)-2-methyl-3-(((R)-10-methyl-11-oxo-2,3,10,11-tetrahydro-1H,5H-benzo[d]pyrazolo[1,2-a][1,2]diazepin-10-yl)amino)-3-oxopropyl)thiazole-2,5-dicarboxamide

To a mixture of (2R)-3-amino-2-methyl-N-(10-methyl-11-oxo-2,3,10,11-tetrahydro-1H,5H-benzo[d]pyrazolo[1,2-a][1,2]diazepin-10-yl)propanamide (Step 5) and 4-methyl-2-(methylcarbamoyl)thiazole-5-carboxylic acid (158 mg, 0.790 mmol) in CH₂Cl₂ (4 mL) and pyridine (2.5 mL) was added EDC (454 mg, 2.37 mmol) and the reaction mixture was stirred at 40° C. for 3 h. After cooling to rt, the reaction mixture was concentrated in vacuo. The crude residue was diluted with a 5% v/v solution of acetic acid in acetonitrile (40 mL) and the diastereomeric mixture was filtered and purified by prep-HPLC (Sunfire C18 column, eluting with a gradient of acetonitrile/water containing 0.1% TFA, at flow rate of 60 mL/min) to afford each diastereomer as its TFA salt.

Example 3: Retention time on LC-MS tᵣ=0.94 min, LC-MS calculated for C₂₄H₃₁N₆O₄S (M+H)⁺: m/z=499.2; found 499.2. ¹H NMR (500 MHZ, DMSO-d₆) δ 8.83 (q, J=4.7 Hz, 1H), 8.29 (t, J=5.6 Hz, 1H), 8.19 (s, 1H), 7.49 (d, J=8.1 Hz, 1H), 7.14 (t, J=7.4 Hz, 1H), 7.06 (t, J=7.7 Hz, 1H), 7.00 (d, J=7.3 Hz, 1H), 4.21 (d, J=15.9 Hz, 1H), 3.99 (d, J=15.9 Hz, 1H), 3.63-3.54 (m, 1H), 3.54-3.46 (m, 1H), 3.37-3.27 (m, 1H), 3.27-3.12 (m, 3H), 2.84-2.67 (m, 4H), 2.59 (s, 3H), 2.20-2.00 (m, 2H), 1.81 (s, 3H), 1.07 (d, J=7.0 Hz, 3H).

Example 4: Retention time on LC-MS tᵣ=0.99 min, LC-MS calculated for C₂₄H₃₁N₆O₄S (M+H)⁺: m/z=499.2; found 499.2. ¹H NMR (600 MHz, DMSO-d₆) δ 8.83 (q, J=4.7 Hz, 1H), 8.50-8.36 (m, 2H), 7.51-7.43 (m, 1H), 7.18-7.12 (m, 2H), 7.04-6.99 (m, 1H), 4.25 (d, J=15.8 Hz, 1H), 3.99 (d, J=15.9 Hz, 1H), 3.66-3.58 (m, 1H), 3.54-3.44 (m, 2H), 3.26-3.17 (m, 2H), 3.17-3.08 (m, 1H), 2.79 (d, J=4.7 Hz, 3H), 2.75-2.67 (m, 1H), 2.62 (s, 3H), 2.23-2.12 (m, 1H), 2.12-2.02 (m, 1H), 1.79 (s, 3H), 0.97 (d, J=6.8 Hz, 3H).

### Examples 5 and 6. N⁵—((R)-3-(((S)-10-Benzyl-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide and N⁵—((R)-3-(((R)-10-benzyl-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide

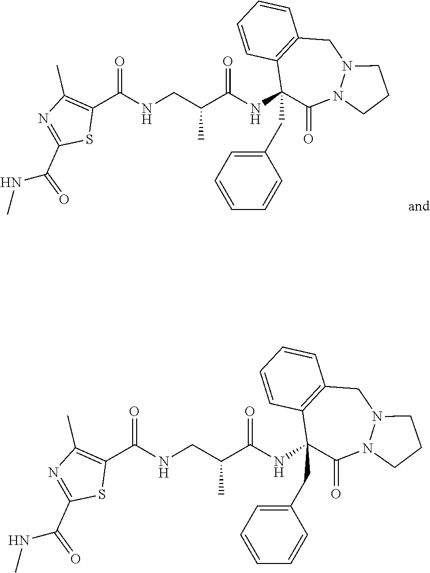 [SMILES: CNC(=O)c1nc(C)c(C(=O)NC[C@@H](C)C(=O)N[C@]2(Cc3ccccc3)C(=O)N3CCCN3Cc3ccccc32)s1 ; CNC(=O)c1nc(C)c(C(=O)NC[C@@H](C)C(=O)N[C@@]2(Cc3ccccc3)C(=O)N3CCCN3Cc3ccccc32)s1]

These compounds were prepared according to the procedures described in Examples 3 and 4, with (bromomethyl)benzene replacing iodomethane in Step 2. The diastereomeric mixture was filtered and purified by prep-HPLC (Sunfire C18 column, eluting with a gradient of acetonitrile/water containing 0.1% TFA, at flow rate of 60 mL/min) to afford each diastereomer as its TFA salt.

Example 5: Retention time on LC-MS tᵣ=1.14 min, LC-MS calculated for C₃₀H₃₅N₆O₄S (M+H)⁺: m/z=575.2; found 575.3.

Example 6: Retention time on LC-MS tᵣ=1.17 min, LC-MS calculated for C₃₀H₃₅N₆O₄S (M+H)⁺: m/z=575.2; found 575.3.

### Examples 7 and 8. N⁵—((R)-3-(((S)-10-Ethyl-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide and N⁵—((R)-3-(((R)-10-ethyl-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide

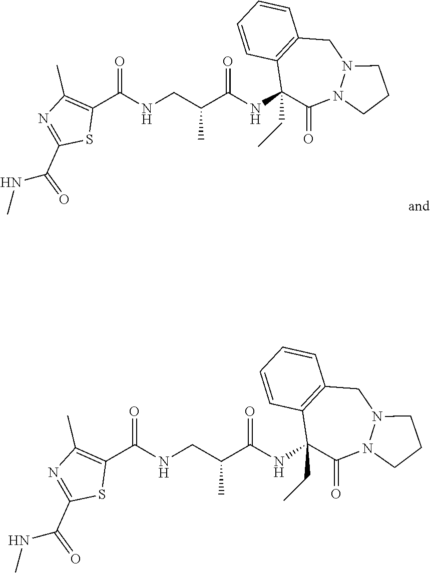 [SMILES: CC[C@@]1(NC(=O)[C@H](C)CNC(=O)c2sc(C(=O)NC)nc2C)C(=O)N2CCCN2Cc2ccccc21 ; CC[C@]1(NC(=O)[C@H](C)CNC(=O)c2sc(C(=O)NC)nc2C)C(=O)N2CCCN2Cc2ccccc21]

These compounds were prepared according to the procedures described in Examples 3 and 4, with iodoethane replacing iodomethane in Step 2. The diastereomeric mixture was filtered and purified by prep-HPLC (Sunfire C18 column, eluting with a gradient of acetonitrile/water containing 0.1% TFA, at flow rate of 60 mL/min) to afford each diastereomer as its TFA salt.

Example 7: Retention time on LC-MS tᵣ=0.946 min, LC-MS calculated for C₂₅H₃₃N₆O₄S (M+H)⁺: m/z=513.2; found 513.3.

Example 8: Retention time on LC-MS tᵣ=0.998 min, LC-MS calculated for C₂₅H₃₃N₆O₄S (M+H)⁺: m/z=513.2; found 513.3.

### Examples 9 and 10. N²,4-Dimethyl-N⁵—((R)-2-methyl-3-oxo-3-(((S)-11-oxo-10-(pyridin-3-ylmethyl)-2,3,10,11-tetrahydro-1H,5H-benzo[d]pyrazolo[1,2-a][1,2]diazepin-10-yl)amino)propyl)thiazole-2,5-dicarboxamide and N²,4-dimethyl-N⁵—((R)-2-methyl-3-oxo-3-(((R)-11-oxo-10-(pyridin-3-ylmethyl)-2,3,10,11-tetrahydro-1H,5H-benzo[d]pyrazolo[1,2-a][1,2]diazepin-10-yl)amino)propyl)thiazole-2,5-dicarboxamide

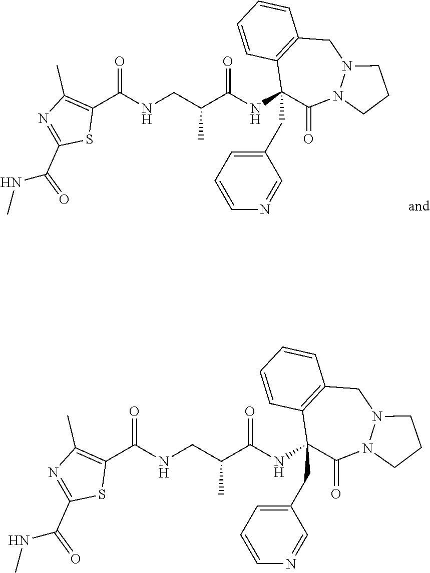 [SMILES: CNC(=O)c1nc(C)c(C(=O)NC[C@@H](C)C(=O)N[C@]2(Cc3cccnc3)C(=O)N3CCCN3Cc3ccccc32)s1 ; CNC(=O)c1nc(C)c(C(=O)NC[C@@H](C)C(=O)N[C@@]2(Cc3cccnc3)C(=O)N3CCCN3Cc3ccccc32)s1]

These compounds were prepared according to the procedures described in Examples 3 and 4, with 3-(bromomethyl)pyridine hydrobromide (Combi-Blocks ST-7536) replacing iodomethane in Step 2. The diastereomeric mixture was filtered and purified by prep-HPLC (Sunfire C18 column, eluting with a gradient of acetonitrile/water containing 0.1% TFA, at flow rate of 60 mL/min) to afford each diastereomer as its TFA salt.

Example 9: Retention time on LC-MS tᵣ=0.794 min, LC-MS calculated for C₂₉H₃₄N—O₄S (M+H)⁺: m/z=576.2; found 576.3.

Example 10: Retention time on LC-MS tᵣ=0.824 min, LC-MS calculated for C₂₉H₃₄N—O₄S (M+H)⁺: m/z=576.2; found 576.3.

### Examples 11 and 12. N⁵—((R)-3-(((S)-10-(4-Fluorobenzyl)-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide and N⁵—((R)-3-(((R)-10-(4-fluorobenzyl)-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide

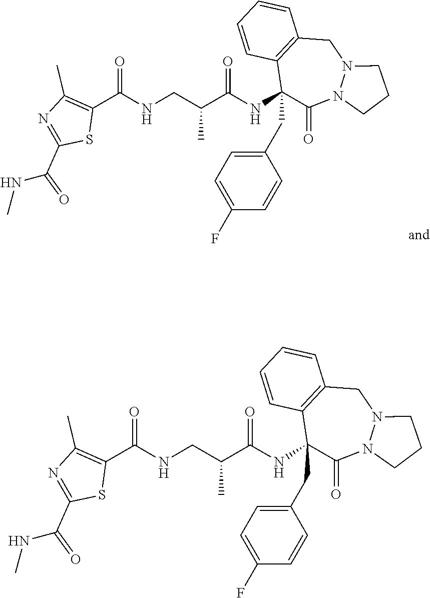 [SMILES: CNC(=O)c1nc(C)c(C(=O)NC[C@@H](C)C(=O)N[C@]2(Cc3ccc(F)cc3)C(=O)N3CCCN3Cc3ccccc32)s1 ; CNC(=O)c1nc(C)c(C(=O)NC[C@@H](C)C(=O)N[C@@]2(Cc3ccc(F)cc3)C(=O)N3CCCN3Cc3ccccc32)s1]

These compounds were prepared according to the procedures described in Examples 3 and 4, with 1-bromomethyl-4-fluorobenzene replacing iodomethane in Step 2. The diastereomeric mixture was filtered and purified by prep-HPLC (Sunfire C18 column, eluting with a gradient of acetonitrile/water containing 0.1% TFA, at flow rate of 60 ml/min) to afford each diastereomer as its TFA salt.

Example 11: Retention time on LC-MS tᵣ=1.26 min, LC-MS calculated for C₃₀H₃₄FN₆O₄S (M+H)⁺: m/z=593.2; found 593.3.

Example 12: Retention time on LC-MS tᵣ=1.29 min, LC-MS calculated for C₃₀H₃₄FN₆O₄S (M+H)⁺: m/z=593.2; found 593.3.

### Examples 13 and 14. N²,4-Dimethyl-N⁵—((R)-2-methyl-3-(((S)-10-(methyl-d₃)-11-oxo-2,3,10,11-tetrahydro-1H,5H-benzo[d]pyrazolo[1,2-a][1,2]diazepin-10-yl)amino)-3-oxopropyl)thiazole-2,5-dicarboxamide and N²,4-dimethyl-N⁵—((R)-2-methyl-3-(((R)-10-(methyl-d₃)-11-oxo-2,3,10,11-tetrahydro-1H,5H-benzo[d]pyrazolo[1,2-a][1,2]diazepin-10-yl)amino)-3-oxopropyl)thiazole-2,5-dicarboxamide

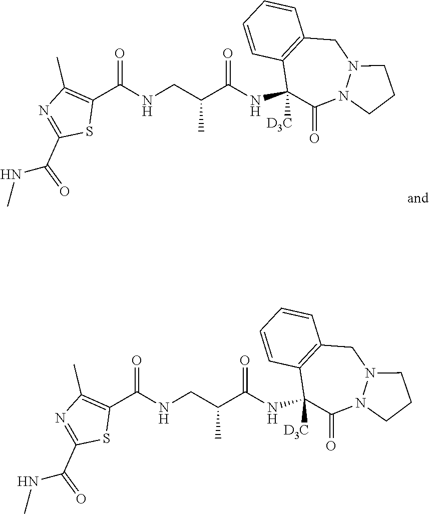 [SMILES: [2H]C([2H])([2H])[C@@]1(NC(=O)[C@H](C)CNC(=O)c2sc(C(=O)NC)nc2C)C(=O)N2CCCN2Cc2ccccc21 ; [2H]C([2H])([2H])[C@]1(NC(=O)[C@H](C)CNC(=O)c2sc(C(=O)NC)nc2C)C(=O)N2CCCN2Cc2ccccc21]

These compounds were prepared according to the procedures described in Examples 3 and 4, with iodomethane-d₃ (Sigma-Aldrich 176036) replacing iodomethane in Step 2. The diastereomeric mixture was filtered and purified by prep-HPLC (Sunfire C18 column, eluting with a gradient of acetonitrile/water containing 0.1% TFA, at flow rate of 60 mL/min) to afford each diastereomer as its TFA salt.

Example 13: Retention time on LC-MS tᵣ=0.942 min, LC-MS calculated for C₂₄H₂₈D₃N₆O₄S (M+H)⁺: m/z=502.2; found 502.2.

Example 14: Retention time on LC-MS tᵣ=0.988 min, LC-MS calculated for C₂₄H₂₈D₃N₆O₄S (M+H)⁺: m/z=502.2; found 502.2.

### Examples 15 and 16. N²,4-Dimethyl-N⁵—((R)-2-methyl-3-oxo-3-(((S)-11-oxo-7-(trifluoromethyl)-2,3,10,11-tetrahydro-1H,5H-benzo[d]pyrazolo[1,2-a][1,2]diazepin-10-yl)amino)propyl)thiazole-2,5-dicarboxamide and N²,4-dimethyl-N⁵—((R)-2-methyl-3-oxo-3-(((R)-11-oxo-7-(trifluoromethyl)-2,3,10,11-tetrahydro-1H,5H-benzo[d]pyrazolo[1,2-a][1,2]diazepin-10-yl)amino)propyl)thiazole-2,5-dicarboxamide

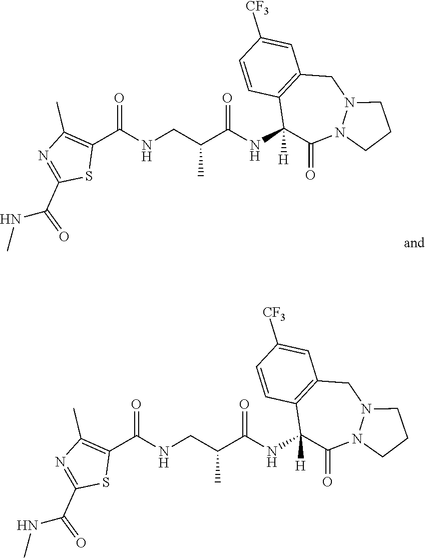 [SMILES: CNC(=O)c1nc(C)c(C(=O)NC[C@@H](C)C(=O)N[C@@H]2C(=O)N3CCCN3Cc3cc(C(F)(F)F)ccc32)s1 ; CNC(=O)c1nc(C)c(C(=O)NC[C@@H](C)C(=O)N[C@H]2C(=O)N3CCCN3Cc3cc(C(F)(F)F)ccc32)s1]

### Step 1:2-(2-Ethoxy-2-oxoethyl)-5-(trifluoromethyl)benzoic acid

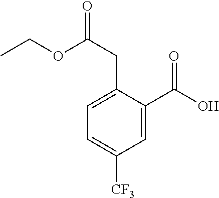 [SMILES: CCOC(=O)Cc1ccc(C(F)(F)F)cc1C(=O)O]

To a mixture of 2-bromo-5-(trifluoromethyl)benzoic acid (5.00 g, 18.6 mmol) in ethanol (50 mL) was added ethyl acetoacetate (4.74 mL, 37.2 mmol), CuBr (3.20 g, 22.3 mmol) and sodium ethoxide (21 wt. % in EtOH, 25 mL, Sigma-Aldrich 230553). The reaction mixture was stirred at reflux for 2 hours. After cooling to rt, the reaction mixture was filtered through a pad of Celite. The filtrate was concentrated in vacuo and the residue was dissolved in 3 M aqueous HCl (75 mL) and extracted with CH₂Cl₂ (3×50 mL). The combined organic layers were washed with brine, dried over Na₂SO₄, and concentrated to afford the desired product (5.1 g, quantitative yield) as a yellow solid. LC-MS calculated for C₁₂H₁₂F₃O₄ (M+H)⁺: m/z=277.2; found 277.2.

### Step 2: Ethyl 2-(2-(hydroxymethyl)-4-(trifluoromethyl)phenyl) acetate

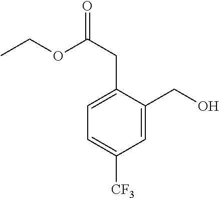 [SMILES: CCOC(=O)Cc1ccc(C(F)(F)F)cc1CO]

To a mixture of 2-(2-ethoxy-2-oxoethyl)-5-(trifluoromethyl)benzoic acid (3.52 g, 12.7 mmol) in CH₂Cl₂ (60 mL) was added triethylamine (3.55 mL, 25.5 mmol) followed by ethyl chlorocarbonate (1.33 mL, 14.0 mmol) and the reaction mixture was stirred at rt for 0.5 h. The reaction mixture was quenched by addition of 1 N aqueous HCl (100 mL) and extracted with CH₂Cl₂ (2×50 mL). The combined organic layers were washed with brine, dried over Na₂SO₄, and concentrated to give the crude intermediate. The crude intermediate was dissolved in 30 mL THF and an aqueous NaBH₄ solution (prepared by dissolving 1 g NaBH₄ in 10 mL water) was added at 0° C. After stirring at 0° C. for 30 min, the reaction was quenched by addition of 1 N aqueous HCl and the aqueous layer was extracted with ethyl acetate (3×30 mL). The combined organic layers were washed with brine, dried over Na₂SO₄, concentrated and the crude residue was purified by flash chromatography (SiO₂, EtOAc/hexanes) to afford the desired product (1.30 g, 39% yield) as a colorless oil. LC-MS calculated for C₁₂H₁₂F302 (M-OH)⁺: m/z=245.1; found 245.2.

### Step 3: Ethyl 2-(2-(((methylsulfonyl)oxy) methyl)-4-(trifluoromethyl)phenyl) acetate

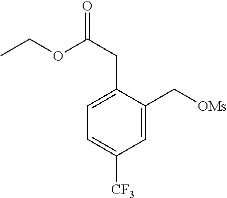 [SMILES: CCOC(=O)Cc1ccc(C(F)(F)F)cc1COS(C)(=O)=O]

To a mixture of ethyl 2-(2-(hydroxymethyl)-4-(trifluoromethyl)phenyl) acetate (1.33 g, 5.07 mmol) in CH₂Cl₂ (30 mL) was added triethylamine (1.41 mL, 10.1 mmol) and methanesulfonyl chloride (0.47 mL, 6.1 mmol). After stirring at rt for 0.5 h, the reaction mixture was quenched by addition of 1 N aqueous HCl and extracted with CH₂Cl₂ (2×20 mL). The combined organic layers were washed with brine, dried over Na₂SO₄, concentrated and the crude residue was purified by flash chromatography (SiO₂, EtOAc/hexanes) to afford the desired product (0.94 g, 55% yield) as a colorless oil. LC-MS calculated for C₁₃H₁₉F₃NO₅S (M+NH₄)+: m/z=358.1; found 358.1.

### Step 4:7-(Trifluoromethyl)-2,3,5, 10-tetrahydro-1H,11H-benzo[d]pyrazolo[1,2-a][1,2]diazepin-11-one

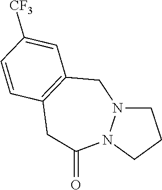 [SMILES: O=C1Cc2ccc(C(F)(F)F)cc2CN2CCCN12]

A mixture of ethyl 2-(2-(hydroxymethyl)-4-(trifluoromethyl)phenyl) acetate (940 mg, 2.76 mmol), pyrazolidine dihydrochloride (400 mg, 2.76 mmol), DIPEA (1.9 mL, 11 mmol) and sodium acetate (906 mg, 11.0 mmol) in DMF (10 mL) was heated to 100° C. for 1 hour. The reaction was diluted with 100 mL ethyl acetate and washed with water (2×50 mL). The organic layer was washed with brine, dried over Na₂SO₄, concentrated and the crude residue was purified by flash chromatography (SiO₂, EtOAc/hexanes) to afford the desired product (0.38 g, 51% yield) as a colorless oil. LC-MS calculated for C₁₃H₁₄F₃N₂O (M+H)⁺: m/z=271.1; found 271.2.

### Step 5:10-Amino-7-(trifluoromethyl)-2,3,5,10-tetrahydro-1H,11H-benzo[d]pyrazolo[1,2-a][1,2]diazepin-11-one

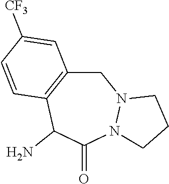 [SMILES: NC1C(=O)N2CCCN2Cc2cc(C(F)(F)F)ccc21]

To a mixture of 7-(trifluoromethyl)-2,3,5,10-tetrahydro-1H,11H-benzo[d]pyrazolo[1,2-a][1,2]diazepin-11-one (0.38 g, 1.4 mmol) and isopentyl nitrite (0.28 mL, 2.1 mmol) in THF (14 mL) was added LiHMDS (1 M in THF, 3.1 mL, 3.1 mmol, Sigma-Aldrich 225770) at 0° C. After stirring at 0° C. for 2 hours, the reaction mixture was quenched by addition of water and extracted with ethyl acetate. The combined organic layers were washed with brine, dried over Na₂SO₄, and concentrated to give the crude intermediate. The crude intermediate was dissolved in acetic acid (5 mL) and 3 N aqueous HCl (1 mL). Zinc powder (800 mg, 12.2 mmol) was added, and the reaction mixture was allowed to stir at rt for 1 h. After completion of reaction, the reaction mixture was filtered, and the filtrate was diluted with 10% w/w aqueous NaOH solution and extracted with CH₂Cl₂. The combined organic layers were washed with brine, dried over Na₂SO₄, and concentrated to afford the desired product (0.30 g, 75% yield) as a yellow oil. LC-MS calculated for C₁₃H₁₅F₃N₃₀ (M+H)⁺: m/z=286.1; found 286.2.

### Step 6: tert-Butyl ((2R)-2-methyl-3-oxo-3-((11-oxo-7-(trifluoromethyl)-2,3,10,11-tetrahydro-1H,5H-benzo[d]pyrazolo[1,2-a][1,2]diazepin-10-yl)amino)propyl)carbamate

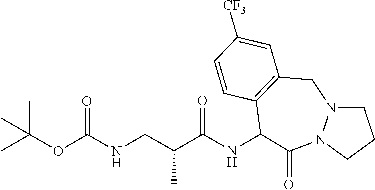 [SMILES: C[C@H](CNC(=O)OC(C)(C)C)C(=O)NC1C(=O)N2CCCN2Cc2cc(C(F)(F)F)ccc21]

To a mixture of 10-amino-7-(trifluoromethyl)-2,3,5,10-tetrahydro-1H,11H-benzo[d]pyrazolo[1,2-a][1,2]diazepin-11-one (150 mg, 0.526 mmol) and (R)-3-((tert-butoxycarbonyl)amino)-2-methylpropanoic acid (128 mg, 0.631 mmol, Combi-Blocks QB-0994) in CH₂Cl₂ (5.3 mL) and pyridine (1.7 mL) was added EDC (302 mg, 1.58 mmol) and the reaction mixture was stirred at 40° C. for 3 h. After cooling to rt, the reaction mixture was concentrated in vacuo. The crude residue was purified by flash chromatography (SiO₂, EtOAc/hexanes) to afford the title compound (195 mg, 79% yield) as a mixture of diastereomers in the form of a colorless oil. LC-MS calculated for C₂₂H₃₀F₃N₄₀₄ (M+H)⁺: m/z=471.2; found 471.2.

### Step 7: (2R)-3-Amino-2-methyl-N-(11-oxo-7-(trifluoromethyl)-2,3,10,11-tetrahydro-1H,5H-benzo[d]pyrazolo[1,2-a][1,2]diazepin-10-yl)propanamide

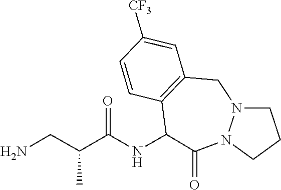 [SMILES: C[C@H](CN)C(=O)NC1C(=O)N2CCCN2Cc2cc(C(F)(F)F)ccc21]

To a mixture tert-butyl ((2R)-2-methyl-3-oxo-3-((11-oxo-7-(trifluoromethyl)-2,3,10,11-tetrahydro-1H,5H-benzo[d]pyrazolo[1,2-a][1,2]diazepin-10-yl)amino)propyl)carbamate (195 mg, 0.414 mmol) in CH₂Cl₂ (10 mL) was added a 4 molar solution of HCl in dioxane (2 mL, 8 mmol, Sigma-Aldrich 345547) and the reaction mixture was stirred at rt for 2 h. The reaction mixture was concentrated to give a crude colorless oil containing the desired product as a mixture of diastereomers (as HCl salt). The crude material obtained was used directly without further purification. LC-MS calculated for C₁₇H₂₂F₃N₄₀₂ (M+H)⁺: m/z=371.2; found 371.2.

### Step 8: N²,4-Dimethyl-N⁵—((R)-2-methyl-3-oxo-3-(((S)-11-oxo-7-(trifluoromethyl)-2,3,10,11-tetrahydro-1H,5H-benzo[d]pyrazolo[1,2-a][1,2]diazepin-10-yl)amino)propyl)thiazole-2,5-dicarboxamide; dicarboxamide and N²,4-dimethyl-N⁵—((R)-2-methyl-3-oxo-3-(((R)-11-oxo-7-(trifluoromethyl)-2,3,10,11-tetrahydro-1H,5H-benzo[d]pyrazolo[1,2-a][1,2]diazepin-10-yl)amino)propyl)thiazole-2,5-dicarboxamide

To a mixture of (2R)-3-amino-2-methyl-N-(11-oxo-7-(trifluoromethyl)-2,3,10,11-tetrahydro-1H,5H-benzo[d]pyrazolo[1,2-a][1,2]diazepin-10-yl)propanamide (Step 7) and 4-methyl-2-(methylcarbamoyl)thiazole-5-carboxylic acid (100 mg, 0.50 mmol) in CH₂Cl₂ (4 mL) and pyridine (1.6 mL) was added EDC (288 mg, 1.50 mmol) and the reaction mixture was stirred at 40° C. for 3 h. After cooling to rt, the reaction mixture was concentrated in vacuo. The crude residue was diluted with a 5% v/v solution of acetic acid in acetonitrile (40 mL) and the diastereomeric mixture was filtered and purified by prep-HPLC (Sunfire C18 column, eluting with a gradient of acetonitrile/water containing 0.1% TFA, at flow rate of 60 mL/min) to afford each diastereomer as its TFA salt.

Example 15: Retention time on LC-MS tᵣ=1.032 min, LC-MS calculated for C₂₄H₂₈F₃N₆O₄S (M+H)⁺: m/z=553.2; found 553.2.

Example 16: Retention time on LC-MS tᵣ=1.093 min, LC-MS calculated for C₂₄H₂₈F₃N₆O₄S (M+H)⁺: m/z=553.2; found 553.2.

### Examples 17 and 18. N²,4-Dimethyl-N⁵—((R)-2-methyl-3-oxo-3-(((S)-11-oxo-7-(trifluoromethyl)-2,3,10,11-tetrahydro-1H,5H-benzo[d]pyrazolo[1,2-a][1,2]diazepin-10-yl-10-d)amino)propyl)thiazole-2,5-dicarboxamide and N²,4-dimethyl-N⁵—((R)-2-methyl-3-oxo-3-(((R)-11-oxo-7-(trifluoromethyl)-2,3,10,11-tetrahydro-1H,5H-benzo[d]pyrazolo[1,2-a][1,2]diazepin-10-yl-10-d)amino)propyl)thiazole-2,5-dicarboxamide

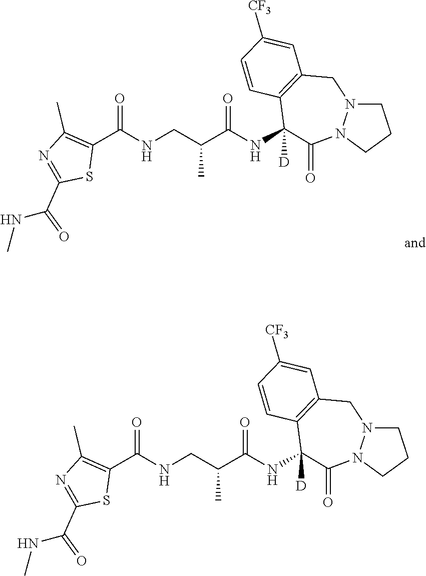 [SMILES: [2H][C@@]1(NC(=O)[C@H](C)CNC(=O)c2sc(C(=O)NC)nc2C)C(=O)N2CCCN2Cc2cc(C(F)(F)F)ccc21 ; [2H][C@]1(NC(=O)[C@H](C)CNC(=O)c2sc(C(=O)NC)nc2C)C(=O)N2CCCN2Cc2cc(C(F)(F)F)ccc21]

These compounds were prepared according to the procedures described in Examples 1 and 2, with Example 16 replacing Example A. The diastereomeric mixture was filtered and purified by prep-HPLC (Sunfire C18 column, eluting with a gradient of acetonitrile/water containing 0.1% TFA, at flow rate of 60 mL/min) to afford each diastereomer as its TFA salt.

Example 17: Retention time on LC-MS tᵣ=1.107 min, LC-MS calculated for C₂₄H₂₇DF₃N₆OS (M+H)⁺: m/z=554.2; found 554.2.

Example 18: Retention time on LC-MS tᵣ=1.171 min, LC-MS calculated for C₂₄H₂₇DF₃N₆O₄S (M+H)⁺: m/z=554.2; found 554.2.

### Examples 19 and 20. N⁵—((R)-3-(((S)-7-Methoxy-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide and N⁵—((R)-3-(((R)-7-methoxy-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide

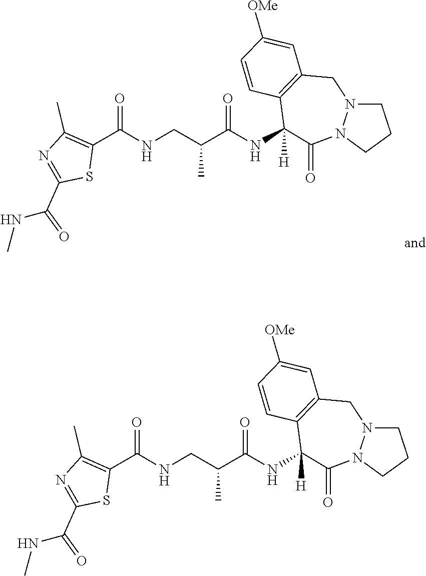 [SMILES: CNC(=O)c1nc(C)c(C(=O)NC[C@@H](C)C(=O)N[C@@H]2C(=O)N3CCCN3Cc3cc(OC)ccc32)s1 ; CNC(=O)c1nc(C)c(C(=O)NC[C@@H](C)C(=O)N[C@H]2C(=O)N3CCCN3Cc3cc(OC)ccc32)s1]

These compounds were prepared according to the procedures described in Examples 15 and 16, with 2-bromo-5-methoxybenzoic acid replacing 2-bromo-5-(trifluoromethyl)benzoic acid in Step 1. The diastereomeric mixture was filtered and purified by prep-HPLC (Sunfire C18 column, eluting with a gradient of acetonitrile/water containing 0.1% TFA, at flow rate of 60 mL/min) to afford each diastereomer as its TFA salt.

Example 19: Retention time on LC-MS tᵣ=1.501 min, LC-MS calculated for C₂₄H₃₁N₆O₅S (M+H)⁺: m/z=515.2; found 515.2.

Example 20: Retention time on LC-MS tᵣ=1.560 min, LC-MS calculated for C₂₄H₃₁N₆O₅S (M+H)⁺: m/z=515.2; found 515.2.

### Examples B and C. N⁵—((R)-3-(((S)-7-Fluoro-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide and N⁵—((R)-3-(((R)-7-fluoro-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide

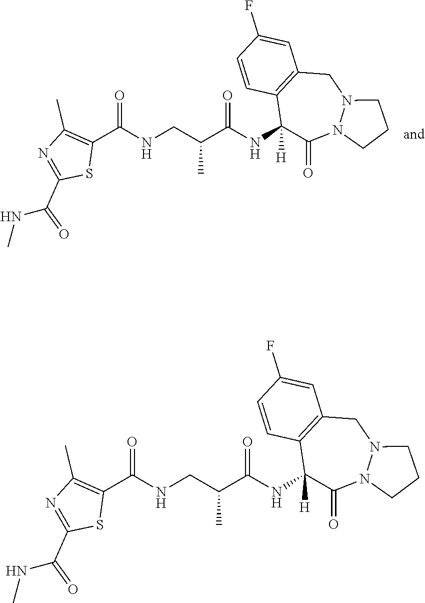 [SMILES: CNC(=O)c1nc(C)c(C(=O)NC[C@@H](C)C(=O)N[C@@H]2C(=O)N3CCCN3Cc3cc(F)ccc32)s1 ; CNC(=O)c1nc(C)c(C(=O)NC[C@@H](C)C(=O)N[C@H]2C(=O)N3CCCN3Cc3cc(F)ccc32)s1]

These compounds were prepared according to the procedures described in Examples 15 and 16, with 2-bromo-5-fluorobenzoic acid replacing 2-bromo-5-(trifluoromethyl)benzoic acid in Step 1. The diastereomeric mixture was filtered and purified by prep-HPLC (Sunfire C18 column, eluting with a gradient of acetonitrile/water containing 0.1% TFA, at flow rate of 60 mL/min) to afford each diastereomer as its TFA salt.

Example B: Retention time on LC-MS tᵣ=0.955 min, LC-MS calculated for C₂₃H₂₈FN₆O₄S (M+H)⁺: m/z=503.2; found 503.2.

Example C: Retention time on LC-MS tᵣ=1.007 min, LC-MS calculated for C₂₃H₂₈FN₆O₄S (M+H)⁺: m/z=503.2; found 503.2.

### Examples 21 and 22. N⁵—((R)-3-(((S)-7-Fluoro-11-oxo-2,3,10,11-tetrahydro-1H,5H-benzo[d]pyrazolo[1,2-a][1,2]diazepin-10-yl-10-d)amino)-2-methyl-3-oxopropyl)-N²,4-dimethylthiazole-2,5-dicarboxamide and N⁵—((R)-3-(((R)-7-fluoro-11-oxo-2,3,10,11-tetrahydro-1H,5H-benzo[d]pyrazolo[1,2-a][1,2]diazepin-10-yl-10-d)amino)-2-methyl-3-oxopropyl)-N²,4-dimethylthiazole-2,5-dicarboxamide

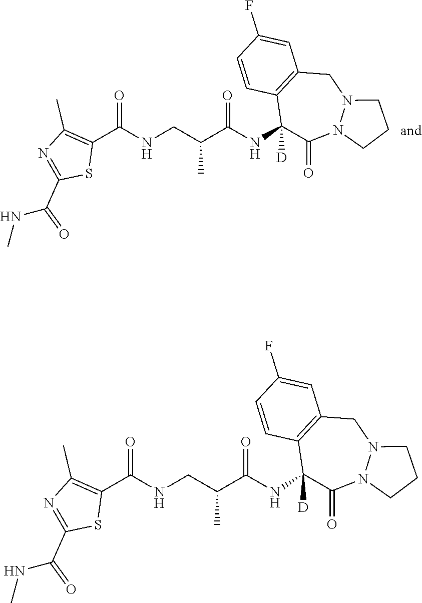 [SMILES: [2H][C@@]1(NC(=O)[C@H](C)CNC(=O)c2sc(C(=O)NC)nc2C)C(=O)N2CCCN2Cc2cc(F)ccc21 ; [2H][C@]1(NC(=O)[C@H](C)CNC(=O)c2sc(C(=O)NC)nc2C)C(=O)N2CCCN2Cc2cc(F)ccc21]

These compounds were prepared according to the procedures described in Examples 1 and 2, with Example C replacing Example A. The diastereomeric mixture was filtered and purified by prep-HPLC (Sunfire C18 column, eluting with a gradient of acetonitrile/water containing 0.1% TFA, at flow rate of 60 mL/min) to afford each diastereomer as its TFA salt.

Example 21: Retention time on LC-MS tᵣ=0.953 min, LC-MS calculated for C₂₃H₂₇DFN₆OS (M+H)⁺: m/z=504.2; found 504.2.

Example 22: Retention time on LC-MS tᵣ=1.005 min, LC-MS calculated for C₂₃H₂₇DFN₆O₄S (M+H)⁺: m/z=504.2; found 504.2.

### Examples D and E. N⁵—((R)-3-(((S)-7-C₆ oro-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide and N⁵—((R)-3-(((R)-7-chloro-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide

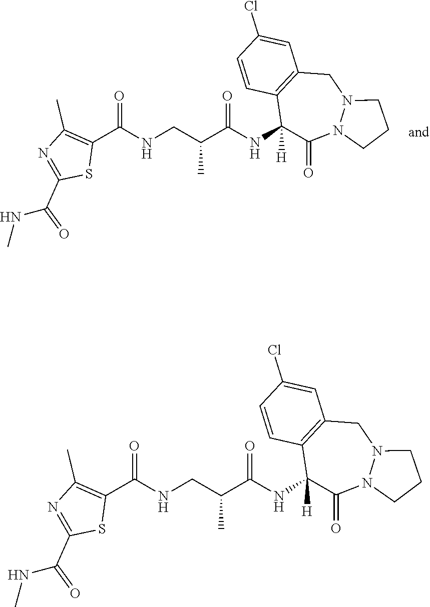 [SMILES: CNC(=O)c1nc(C)c(C(=O)NC[C@@H](C)C(=O)N[C@@H]2C(=O)N3CCCN3Cc3cc(Cl)ccc32)s1 ; CNC(=O)c1nc(C)c(C(=O)NC[C@@H](C)C(=O)N[C@H]2C(=O)N3CCCN3Cc3cc(Cl)ccc32)s1]

These compounds were prepared according to the procedures described in Examples 15 and 16, with 2-bromo-5-chlorobenzoic acid replacing 2-bromo-5-(trifluoromethyl)benzoic acid in Step 1. The diastereomeric mixture was filtered and purified by prep-HPLC (Sunfire C18 column, eluting with a gradient of acetonitrile/water containing 0.1% TFA, at flow rate of 60 mL/min) to afford each diastereomer as its TFA salt.

Example D: Retention time on LC-MS tᵣ=1.029 min, LC-MS calculated for C₂₃H₂₈ClN₆O₄S (M+H)⁺: m/z=519.2; found 519.2.

Example E: Retention time on LC-MS tᵣ=1.096 min, LC-MS calculated for C₂₃H₂₈ClN₆O₄S (M+H)⁺: m/z=519.2; found 519.2.

### Examples 23 and 24. N⁵—((R)-3-(((S)-7-C₆ oro-11-oxo-2,3,10,11-tetrahydro-1H,5H-benzo[d]pyrazolo[1,2-a][1,2]diazepin-10-yl-10-d)amino)-2-methyl-3-oxopropyl)-N²,4-dimethylthiazole-2,5-dicarboxamide and N⁵—((R)-3-(((R)-7-chloro-11-oxo-2,3,10,11-tetrahydro-1H,5H-benzo[d]pyrazolo[1,2-a][1,2]diazepin-10-yl-10-d)amino)-2-methyl-3-oxopropyl)-N²,4-dimethylthiazole-2,5-dicarboxamide

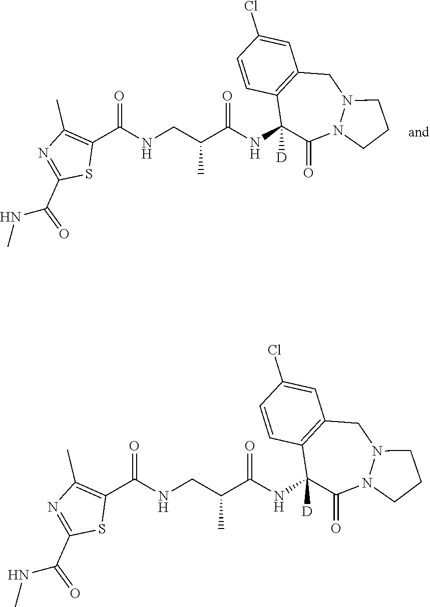 [SMILES: [2H][C@@]1(NC(=O)[C@H](C)CNC(=O)c2sc(C(=O)NC)nc2C)C(=O)N2CCCN2Cc2cc(Cl)ccc21 ; [2H][C@]1(NC(=O)[C@H](C)CNC(=O)c2sc(C(=O)NC)nc2C)C(=O)N2CCCN2Cc2cc(Cl)ccc21]

These compounds were prepared according to the procedures described in Examples 1 and 2, with Example E replacing Example A. The diastereomeric mixture was filtered and purified by prep-HPLC (Sunfire C18 column, eluting with a gradient of acetonitrile/water containing 0.1% TFA, at flow rate of 60 mL/min) to afford each diastereomer as its TFA salt.

Example 23: Retention time on LC-MS tᵣ=1.029 min, LC-MS calculated for C₂₃H₂₈ClN₆O₄S (M+H)⁺: m/z=520.2; found 520.2.

Example 24: Retention time on LC-MS tᵣ=1.096 min, LC-MS calculated for C₂₃H₂₈ClN₆O₄S (M+H)⁺: m/z=520.2; found 520.2.

### Example 25. 4-Methyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl)amino)propyl)thiazole-2,5-dicarboxamide

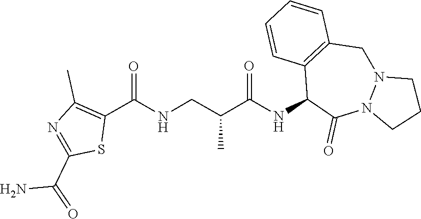 [SMILES: Cc1nc(C(N)=O)sc1C(=O)NC[C@@H](C)C(=O)N[C@@H]1C(=O)N2CCCN2Cc2ccccc21]

This compound was prepared according to the procedures described in Example A, with 2-carbamoyl-4-methylthiazole-5-carboxylic acid (int-IN1) replacing 4-methyl-2-(methylcarbamoyl)thiazole-5-carboxylic acid (int-EC4) in Step 3. The crude product was filtered and purified by prep-HPLC (Sunfire C18 column, eluting with a gradient of acetonitrile/water containing 0.1% TFA, at flow rate of 60 mL/min) to afford the desire product as its TFA salt. LC-MS calculated for C₂₂H₂₇N₆O₄S (M+H)⁺: m/z=471.2; found 471.2. 1H NMR (500 MHz, DMSO-d₆) δ 8.44-8.32 (m, 2H), 8.21 (s, 1H), 7.92 (s, 1H), 7.23 (d, J=7.9 Hz, 1H), 7.18 (t, J=7.4 Hz, 1H), 7.05 (d, J=7.6 Hz, 1H), 6.92 (t, J=7.6 Hz, 1H), 6.73 (d, J=8.8 Hz, 1H), 4.24 (s, 2H), 3.60-3.44 (m, 3H), 3.34-3.25 (m, 2H), 3.22-3.14 (m, 1H), 3.13-3.03 (m, 1H), 2.59 (s, 3H), 2.41-2.30 (m, 1H), 2.15-2.05 (m, 1H), 1.10 (d, J=6.9 Hz, 3H).

### Examples 26 and 27. 4-Methyl-N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide and 4-methyl-N⁵—((R)-2-methyl-3-oxo-3-(((R)-11-oxo-2,3,10,11-tetrahydro-1H,5H-benzo[d]pyrazolo[1,2-a][1,2]diazepin-10-yl-10-d)amino)propyl)thiazole-2,5-dicarboxamide

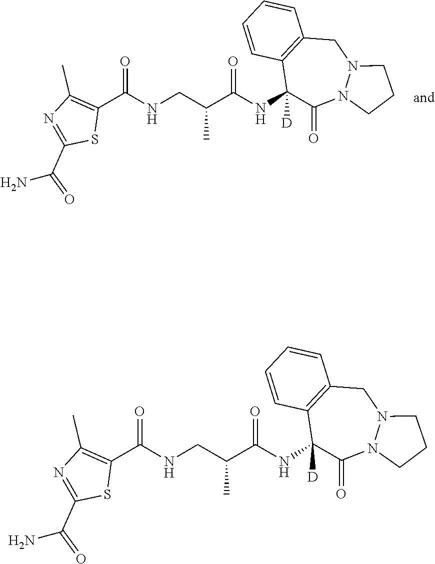 [SMILES: [2H][C@@]1(NC(=O)[C@H](C)CNC(=O)c2sc(C(N)=O)nc2C)C(=O)N2CCCN2Cc2ccccc21 ; [2H][C@]1(NC(=O)[C@H](C)CNC(=O)c2sc(C(N)=O)nc2C)C(=O)N2CCCN2Cc2ccccc21]

These compounds were prepared according to the procedures described in Examples 1 and 2, with Example 25 replacing Example A. The diastereomeric mixture was filtered and purified by prep-HPLC (Sunfire C18 column, eluting with a gradient of acetonitrile/water containing 0.1% TFA, at flow rate of 60 mL/min) to afford each diastereomer as its TFA salt.

Example 26: Retention time on LC-MS tᵣ=0.849 min, LC-MS calculated for C₂₂H₂₆DN₆O₄S (M+H)⁺: m/z=472.2; found 472.2. ¹H NMR (600 MHZ, DMSO-d₆) δ 8.46-8.32 (m, 2H), 8.22 (s, 1H), 7.92 (s, 1H), 7.22 (d, J=8.0 Hz, 1H), 7.18 (t, J=7.4 Hz, 1H), 7.05 (d, J=7.6 Hz, 1H), 6.91 (t, J=7.6 Hz, 1H), 4.24 (s, 2H), 3.59-3.43 (m, 3H), 3.33-3.24 (m, 2H), 3.23-3.14 (m, 1H), 3.13-3.04 (m, 1H), 2.58 (s, 3H), 2.41-2.30 (m, 1H), 2.16-2.06 (m, 1H), 1.10 (d, J=7.0 Hz, 3H).

Example 27: Retention time on LC-MS tᵣ=0.909 min, LC-MS calculated for C₂₂H₂₆DN₆O₄S (M+H)⁺: m/z=472.2; found 472.2. ¹H NMR (600 MHZ, DMSO-d₆) δ 8.49-8.37 (m, 2H), 8.19 (s, 1H), 7.91 (s, 1H), 7.27-7.17 (m, 3H), 7.07 (d, J=7.6 Hz, 1H), 4.30-4.20 (m, 2H), 3.57-3.46 (m, 2H), 3.44-3.23 (m, 3H), 3.22-3.13 (m, 1H), 3.11-3.02 (m, 1H), 2.56 (s, 3H), 2.41-2.30 (m, 1H), 2.14-2.04 (m, 1H), 1.16 (d, J=6.9 Hz, 3H).

### Example 28. 4-Methyl-N₂-(methyl-d₃)—N⁵—((R)-2-methyl-3-oxo-3-(((S)-11-oxo-2,3,10,11-tetrahydro-1H,5H-benzo[d]pyrazolo[1,2-a][1,2]diazepin-10-yl)amino)propyl)thiazole-2,5-dicarboxamide

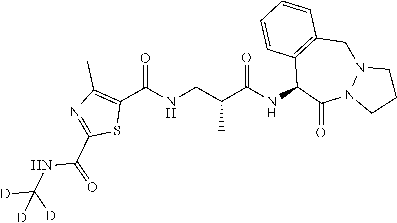 [SMILES: [2H]C([2H])([2H])NC(=O)c1nc(C)c(C(=O)NC[C@@H](C)C(=O)N[C@@H]2C(=O)N3CCCN3Cc3ccccc32)s1]

This compound was prepared according to the procedures described in Example A, with 4-methyl-2-((methyl-d₃)carbamoyl)thiazole-5-carboxylic acid (int-IN2) replacing 4-methyl-2-(methylcarbamoyl)thiazole-5-carboxylic acid (int-EC4) in Step 3. The crude product was filtered and purified by prep-HPLC (Sunfire C18 column, eluting with a gradient of acetonitrile/water containing 0.1% TFA, at flow rate of 60 mL/min) to afford the desire product as its TFA salt.

Example 28: Retention time on LC-MS tᵣ=0.920 min, LC-MS calculated for C₂₃H₂₆D₃N₆O₄S (M+H)⁺: m/z=488.2; found 488.2. ¹H NMR (600 MHZ, DMSO-d₆) δ 8.82 (s, 1H), 8.40 (t, J=5.1 Hz, 1H), 8.37 (d, J=8.8 Hz, 1H), 7.22 (d, J=7.9 Hz, 1H), 7.18 (t, J=7.4 Hz, 1H), 7.05 (d, J=7.6 Hz, 1H), 6.91 (t, J=7.6 Hz, 1H), 6.73 (d, J=8.8 Hz, 1H), 4.24 (s, 2H), 3.61-3.43 (m, 3H), 3.33-3.23 (m, 2H), 3.22-3.14 (m, 1H), 3.12-3.03 (m, 1H), 2.59 (s, 3H), 2.41-2.30 (m, 1H), 2.17-2.04 (m, 1H), 1.10 (d, J=6.9 Hz, 3H).

### Examples 29 and 30. 4-Methyl-N₂-(methyl-d₃)—N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide (29) and 4-methyl-N₂-(methyl-d₃)—N⁵—((R)-2-methyl-3-oxo-3-(((R)-11-oxo-2,3,10,11-tetrahydro-1H,5H-benzo[d]pyrazolo[1,2-a][1,2]diazepin-10-yl-10-d)amino)propyl)thiazole-2,5-dicarboxamide (30)

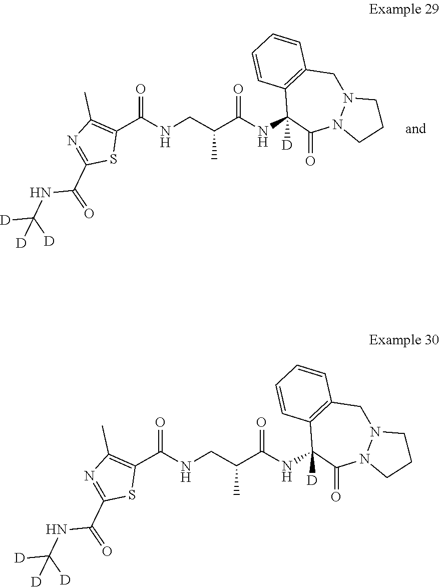 [SMILES: [2H]C([2H])([2H])NC(=O)c1nc(C)c(C(=O)NC[C@@H](C)C(=O)N[C@]2([2H])C(=O)N3CCCN3Cc3ccccc32)s1 ; [2H]C([2H])([2H])NC(=O)c1nc(C)c(C(=O)NC[C@@H](C)C(=O)N[C@@]2([2H])C(=O)N3CCCN3Cc3ccccc32)s1]

These compounds were prepared according to the procedures described in Examples 1 and 2, with Example 28 replacing Example A. The diastereomeric mixture was filtered and purified by prep-HPLC (Sunfire C18 column, eluting with a gradient of acetonitrile/water containing 0.1% TFA, at flow rate of 60 mL/min) to afford each diastereomer as its TFA salt.

Example 29: Retention time on LC-MS tᵣ=0.922 min, LC-MS calculated for C₂₃H₂₅D₄N₆O₄S (M+H)⁺: m/z=489.2; found 489.2. ¹H NMR (600 MHZ, DMSO-d₆) δ 8.82 (s, 1H), 8.40 (t, J=5.6 Hz, 1H), 8.37 (s, 1H), 7.23 (d, J=7.9 Hz, 1H), 7.18 (t, J=7.5 Hz, 1H), 7.05 (d, J=7.6 Hz, 1H), 6.92 (t, J=7.6 Hz, 1H), 4.24 (s, 2H), 3.60-3.43 (m, 3H), 3.33-3.24 (m, 2H), 3.22-3.14 (m, 1H), 3.11-3.03 (m, 1H), 2.59 (s, 3H), 2.42-2.31 (m, 1H), 2.15-2.05 (m, 1H), 1.10 (d, J=7.0 Hz, 3H).

Example 30: Retention time on LC-MS tᵣ=0.976 min, LC-MS calculated for C₂₃H₂₅D₄N₆O₄S (M+H)⁺: m/z=489.2; found 489.2. ¹H NMR (600 MHZ, DMSO-d₆) δ 8.78 (s, 1H), 8.44 (t, J=5.7 Hz, 1H), 8.41 (s, 1H), 7.29-7.16 (m, 3H), 7.06 (d, J=7.5 Hz, 1H), 4.28-4.20 (m, 2H), 3.56-3.47 (m, 2H), 3.41-3.24 (m, 3H), 3.22-3.15 (m, 1H), 3.11-3.03 (m, 1H), 2.57 (s, 3H), 2.40-2.30 (m, 1H), 2.14-2.05 (m, 1H), 1.16 (d, J=6.9 Hz, 3H).

### Example A-1: In Vivo Epimerization Reduction

The chiral carbon in the 7-membered ring of Example A exhibits epimerization in vivo. The resulting diastereomer of Example A has a significantly different PK profile than Example A. For instance, the diastereomer has a prolonged t ½ (half life) and undetermined formation and elimination routes. These factors may lead to accumulation and potenial toxicity. The effect on epimerization by substitution at the chiral carbon was tested by comparing Example A, Example 1, and Example 29 in the following preclinical in vivo pharmacokinetic models: Sprague Dawley rats, beagle dogs, and cynomolgus monkeys.

Two groups of male Sprague Dawley rats (n=3 each) received either a single 10 mg/kg PO dose of Example A or Example 1 in a vehicle consisting of 0.5% methylcellulose in water. Blood samples were collected at 0.25, 0.5, 1, 2, 4, and 6 hours postdose.

Male Sprague Dawley rats (n=3 each) received a single 3 mg/kg PO dose of Example 29 in a vehicle consisting of 5% dimethylacetamide (DMAC) in 50 mM citrate buffer w/0.5% methylcellulose. Blood samples were collected at predose, 0.25, 0.5, 1, 2, 4, 6, 8, 12, and 24 hours postdose.

Three groups of male cynomolgus monkeys (n=2 each) received either a single 3 mg/kg PO dose of Example A, Example 1, or Example 29 in a vehicle consisting of 5% dimethylacetamide (DMAC) in 50 mM citrate buffer w/0.5% methylcellulose. Blood samples were collected at predose, 0.25, 0.5, 1, 2, 3, 4, 6, 8, 12, and 24 hours postdose.

Three groups of male beagle dogs (n=2 each) received either a single 3 mg/kg PO dose of Example A, Example 1, or Example 29 in a vehicle consisting of 5% dimethylacetamide (DMAC) in 50 mM citrate buffer w/0.5% methylcellulose. Blood samples were collected at predose, 0.25, 0.5, 1, 2, 3, 4, 6, 8, 12, 24 and 48 hours postdose.

All blood samples were collected using EDTA as the anticoagulant and centrifuged to obtain plasma. Plasma concentrations of Example A, Example 1, and their respective diastereomers were determined under non-GLP conditions using standard reverse phase LC-MS/MS bioanalytical methods. The individual plasma concentration-time data was used to determine the area under the curve (AUC) values using GraphPad Prism (v9.3.1).

Epimerization of Example A, Example 1, and Example 29 was observed in all three models. Substitution (e.g., deuteration) at the chiral carbon in the 7-membered ring of Example A, however, reduces epimerization. Epimerization with Example 1 was reduced by approximately 6, 11, and 59-fold in rats, monkeys, and dogs, respectively, based on AUC ratios of epimer/parentas shown in Table 1 and FIGS. 1A, 1B, and 1C. Epimerization with Example 29 was reduced by approximately 4.5, 52, and 114-fold in rats, monkeys, and dogs, respectively, based on AUC ratios of epimer/parent (Table 1).

Two additional observations were made. In general, the exposures and PK of Example 1, Example A, and Example 29 (other than epimerization) across species are consistent. The deuterium of Example 1 and Example 29 was retained in the process of epimerization in all species studied, forming Example 2 and Example 30, respectively. Deuteration reduces epimerization by about 6, 10, and >50-fold in rats, monkeys, and dogs, respectively, based on AUC ratios of epimer/parent with Example 1. Deuteration reduces epimerization by about 5, 50, and >100-fold in rats, monkeys, and dogs, respectively, based on AUC ratios of epimer/parent with Example 29.

TABLE 1

| | | | Fold | | Fold |
|---|---|---|---|---|---|
| | Example A | Example 1 | Decrease in | Example 29 | Decrease in |
| | diastereomer | diastereomer | epimerization | diastereomer | epimerization |
| | AUC (%) | AUC (%) | (A/1) | AUC (%) | (A/29) |
| Rat | 4.9 | 0.80 | 6.1 | 1.1 | 4.5 |
| Cyno | 150 | 14 | 11 | 2.9 | 52 |
| Dog | 24 | 0.41 | 59 | 0.21 | 114 |

### Example B-1: Nuclear translocation imaging assay for human SPPL2a

The nuclear translocation imaging assays for human SPPL2a were performed using an SPPL2a reporter U2OS cell line as previously described (Velcicky et al 2018, Zhang et al 2017). These cells stably express human SPPL2a and a doxycycline inducible eGFP TNFα (aa 1-76) NTF fusion protein (SPPL2a substrate). Both Example 1 and Example A were added at indicated concentrations and cells were incubated for 24 h, washed, and fixed. Nuclei were stained with DAPI. Images were acquired on a confocal fluorescence microscope system LSM-800 (Carl Zeiss). Image data was analyzed by using mid-section from Z-stack images of at least 10 individual cells per sample. All data is presented as mean+SEM. Statistical significance was calculated with the ANOVA test using GraphPad Prism™ 10. Image analysis showed that, comparable and significant TNFα-GFP inhibition of nuclear translocation by both Example 1 and Example A (FIG. 2).

### Example B-2

To confirm the cleavage of TNF-α in the U2OS cells that express human SPPL2a, a western blot assay was performed. The same cell line used for the imaging assays were used; these cells express human SPPL2a constitutively and an eGFP-labelled TNFα (aa 1-76) NTF substrate under a doxycycline promotor. In the presence of SPPL2a activity, TNFα-GFP is cleaved at the NTF, resulting in a single 54-56 kDa band (lower MW). If SPPL2a activity is inhibited, TNFα GFP processing is abrogated resulting in a higher molecular weight band (60-62 kDa; complete inhibition) and or both 54-56 kDa and 60-62 kDa bands (incomplete inhibition, smeared appearance) on the blot. Both Example 1 and Example A were added at indicated concentrations, and cells were incubated for 24 h with the inhibitors. Total cell lysated were prepared in ice cold-1X RIPA buffer containing protease inhibitor cocktail (AKR-190, Cell BioLabs). Lysates were collected and quantified for the total protein on NanoDrop using BCA kit (23227, Thermo Scientific). Obtained lysates were processed for the protein bands using Jess Simple western blot system (ProteinSimple), using 12-230 kDa separation module. To detect cleaved and un-cleaved TNFα-GFP fragments, anti-GFP antibody (AF4240, R&D Biosystems) was used as a primary antibody and detected using anti-goat detection module (DM-006, ProteinSimple) according to the manufacturer's protocol. Data was analyzed using ProteinSimple compass software.

Comparable and significant TNFα-GFP cleavage inhibition by both Example 1 and Example A was observed (FIG. 3).

### Example B-3

Dendritic cells are crucial players in the antigen presentation process and inflammation. Previous reports suggested an important role of SPPL2a in dendritic cell mediated inflammatory processes. To validate the functional relevance of SPPL2a inhibitor, a DC-maturation assay was utilized. Human PBMCs were isolated from fresh blood using Histoplaque gradients and plated overnight at 37° C. to allow monocytes to adhere. Non-adherent cells were washed off the following day and DC-differentiation media containing GM-CSF and IL-4 was added and incubated for 5 d. Immature MoDCs were separated and washed. A cocktail of maturation media with SPPL2a inhibitor (at indicated concentrations) was added to the wells and cells were incubated for 24 h. Supernatants were collected and processed to measure TNFα and IL-12 by ELISA. All data is presented as mean+SEM. For comparison of more than two groups, ANOVA was used. All statistical analysis was performed using GraphPad Prism™ 10.

Example A significantly decreased MoDC maturation when compared to the stimulation only condition (FIG. 4). Example A also significantly decreased TNFα secretion and IL-12 secretion, indicating the functional relevance of the SPPL2a inhibition on inflammatory cells such as DCs.

### Example C: Efficacy of SPPL2a inhibition on experimental autoimmune encephalomyelitis (EAE) mouse model

The EAE model was used to evaluate the potential of SPPL2a inhibition in an in vivo mouse model of autoimmune disease. In this study, EAE was induced in female mice, aged 7-9 weeks, using an EAE induction kit (EK-2160, Hooke Labs) containing human recombinant MOG 1 125 antigen emulsified in CFA. EAE severity score was measured on a scale of 0-6 as per the IACUC approved protocol for 20 days. Mice were treated orally with the SPPL2a inhibitor Example A at 15 mg/kg and 30 mg/kg twice daily (first dose given on the same day prior to immunization). Animals were euthanized and tissues, plasma, and serum were collected for further analysis.

Treatment with Example A significantly reduced clinical signs of EAE in mice, starting at day 13 of the treatment (FIG. 5). Both 15 mg/kg and 30 mg/kg doses of Example A showed significant reduction in the disease score compared to the vehicle treated group. Although the 30 mg/kg dose group showed a trend for better disease control, there was no statistically significant difference between the 15 mg/kg and 30 mg/kg doses. Anti-mouse MOG antibodies were measured in the serum of mice using quantitative ELISA kit (AS-55156, AnaSpec). There was a significant reduction in the anti-MOG antibodies in the mice treated with the SPPL2a inhibitor. All data is presented as mean+SEM. For comparison of more than two groups, ANOVA was used. All statistical analysis was performed using GraphPad Prism™ 10 or higher.

Treatment with Example A significantly inhibited total Ig levels in EAE induced mice. Significant reduction of mouse IgG2a, IgG2b, IgG3 and IgM titers was observed in mice treated with 30 mg/kg. Trends to lower Ig titers were observed for IgG2a, IgG2b and IgM levels in the mice treated with 15 mg/kg of Example A, and lower total IgG1 and IgA trend was seen in the mice treated with 30 mg/kg but failed to reach statistical significance.

### Example D: Efficacy of SPPL2a Inhibition on Mouse Model of Systemic Lupus (SLE)

A mouse model of SLE was used to evaluate the treatment potential of SPPL2a inhibition by Example A in vivo in female mice aged 8 weeks. The MRL/MpJ-Faslpr/J (JAX stock #000485) mice used in this study develop a spontaneously accelerated and aggressive lupus-like disease characterized by immune-mediated damage to the organs, and by the presence of circulating auto-Abs against dsDNA, which are serological hallmarks of SLE. Mice were prophylactically treated orally with Example A at 15 mg/kg and 30 mg/kg dose, twice daily after allotment into different groups starting at week 10. As a control, cyclophosphamide at 25 mg/kg dose was given intraperitoneally, twice weekly. Mice were monitored weekly for the clinical sign development, proteinuria, skin lesions and lymphadenopathy. The study was terminated by euthanizing mice at 20 weeks of age following 10 weeks of dosing. All data is presented as mean+SEM. For comparison of more than two groups, ANOVA was used. All statistical analysis was performed using GraphPad Prism™10.

Compared to vehicle control, treatment with Example A significantly reduced anti ds-DNA antibody production in mice. In addition, SPPL2a inhibition effectively reduced proteinuria and skin lesion formation in these mice at both doses tested. Lymphadenopathy was significantly reduced at the highest dose tested. Significant reduction in proteinuria, skin lesional scores and lymphadenopathy was observed in mice treated with Example A. Mouse kidney pathology showed that twice daily oral administration of Example A to mice at 30 mg/kg resulted in a statistically significant decrease in membranoproliferative nephritis, tubular dilation and casts, interstitial inflammation, fibrosis, blood vessel necrosis, and inflammation (FIG. 6).

Example A at 15 mg/kg did not exhibit a test article-related change compared to the vehicle control except in the blood vessels.

Inflammatory cytokine levels (TNFα and IFNγ) and murine immunoglobulin levels (Ig) were measured using MSD® multi-spot assay kits. Example A at 30 mg/kg BID, significantly inhibits antibody production and TNFα levels in mouse serum. No significant changes were observed in TNFα levels with the lower dose of Example A, 15 mg/kg BID. Serum IFNγ levels showed a modest increase with both doses on Example A. Additionally, IgG1, IgG2a, IgG2b, IgG3 and IgM were significantly lower in both Example A treated groups. Levels of IgA were significantly down regulated in mice treated with the 15 mg/kg dose and trended lower with the 30 mg/kg dose.

### Example E: Efficacy of Example 1 and Example a in the Imiquimod (IMQ)-Induced Psoriasis Mouse Model

A comparative study was performed to evaluate the efficacy of the deuterated-inhibitor Example 1 to Example A in the mouse model of psoriasis. Briefly, BALB/c female mice (7-8 week age) were shaved on the dorsal skin and 62.5 mg of IMQ cream was applied daily until day 9. Simultaneously, 15 mg of IMQ cream was applied on one ear daily until day 9. Example 1 and Example A were given orally (PO) at 15 mg/kg, BID. Mice were monitored and clinical signs of psoriasis were recorded (PSI score), both on the dorsal skin (erythema, scaling & thickness) and on the IMQ-applied ear (thickness). All data is presented as mean+SEM. For comparison of more than two groups, ANOVA was used. Pearson product moment correlation was used to test for correlations. All statistical analysis was performed using GraphPad Prism™ 10.

Treatment with either of Example 1 or Example A significantly reduced clinical signs of psoriasis disease in mice, compared to vehicle group. Both Example 1 and Example A alleviate the dorsal PSI scores and ear thickness measurements, and there was no statistically significant difference between compounds. Additionally, inflammatory cytokine levels in the plasma (TNF-α and IL-12p40) were strongly and significantly correlated with the terminal PSI scores, indicating the similarity and effectiveness of Example 1 and Example A in this model.

Mouse TNFα and IL-12 levels were measured in the plasma samples collected on terminal day of the study, indicated a significant reduction in cytokine level upon either Example 1 or Example A treatment.

### Example F: Efficacy of SPPL2a inhibitors Example 1 and Example A in the Collagen-Induced Arthritis (CIA) mouse model

Female DBA-1 mice (8-9 weeks old) were used in the CIA mouse model. This model is widely used to evaluate efficacy of immunomodulatory drugs on autoimmune diseases, such as arthritis. DBA/1 mice develop collagen-induced arthritis (CIA) after immunization with bovine type II collagen emulsified in complete Freund's adjuvant (CFA), followed by a booster dose 21 days later. Both the immunizations were given subcutaneously, approximately 25 mm from the base of the tail on opposite sides. Immunizations were performed under a short duration isoflurane anesthesia. Mice were evaluated for the disease progression up to 45 days. Immunized mice showed signs of CIA around day 15 and disease progressed until termination of the study on day 45. Treatment with Example 1 or Example A at 50 mg/kg dose significantly reduced clinical signs of CIA in mice during the study compared to the vehicle (FIG. 7). Throughout the study, dexamethasone (10 mg/kg) treated group showed significant reduction in the CIA score, compared to the vehicle. Statistical significance was determined using Two-way ANOVA with multiple comparison tests to compare all groups to the vehicle.

1. A compound of Formula (I) or a pharmaceutically acceptable salt or stereoisomer thereof, 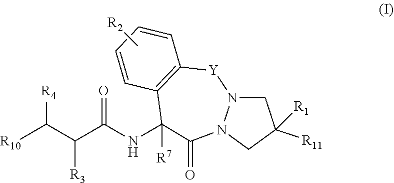
wherein:
Y is CH₂ or C═O;
R₁ is H, C₁-C₆alkyl or halo;
R₂ is H, halo, C₁-C₆alkyl, C₁-C₆haloalkyl, C₁-C₆alkyloxy, and C₁-C₆haloalkyloxy;
R₃ is H, C₁-C₆haloalkyl, Ca-Cecycloalkyl, C₁-C₆alkyl-phenyl or C₁-C₆alkyl optionally substituted with C₁-C₆alkoxy;
R₄ is H, C₁-C₆alkyl or C₁-C₆alkyl-phenyl;
R₁₀ is —NHC(═O)R₅, —C(═O)NHR₅ or a 9 or 10 membered bicyclic heteroaryl having 1 to 4 heteroatoms as ring members each independently selected from N, O and S, wherein the bicyclic heteroaryl is unsubstituted or the bicyclic heteroaryl is substituted with one or more R₆;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by one or more D or C₁-C₆alkoxy;)
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo;
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy; and
xxi) —C(═O)NH₂;
each R₆ is independently selected from C₁-C₆alkyl, C₁-C₆alkoxy, C₁-C₆haloalkyl, cyano and halo;
R₁₁ is H, C₁-C₆alkyl or halo; or
R₁ and R₁₁ together with the carbon atom to which they are attached, may form a 3 to 6 membered carbocyclic ring;
R⁷ is selected from D, CD₃, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, (4-10 membered heterocycloalkyl)-C₁₋₆ alkylene, CN, C(O)R^{b7}, C(O)NR^{c7}R^{d7}, C(O)NR^{c7}(ORd^{d7}), C(O)OR^{d7}, C(═NR^{e7})R^{b7}, C(═NR^{e7})NR^{c7}R^{d7}, S(O)R^{b7}, S(O)NR^{c7}R^{d7}, S(O)₂R^{b7}, and S(O)₂NR^{c7}R^{d7}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R⁷ are each optionally substituted with 1, 2, 3, 4, 5, 6, 7, or 8 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R^{a7}R^{c7}, and R^{d7} are each optionally substituted with 1, 2, 3, 4, 5, 6, 7, or 8 independently selected R^{7A} substituents;
or any R^{c7} and R^{d7} attached to the same N atom, together with the N atom to which they are attached, form a 5-10 membered heteroaryl or a 4-10 membered heterocycloalkyl group, wherein the 5-10 membered heteroaryl or 4-10 membered heterocycloalkyl group is optionally substituted with 1, 2, 3, 4, 5, 6, 7, or 8 independently selected R^{7A} substituents;
each R^{b7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, and (4-10 membered heterocycloalkyl)-C₁₋₆ alkylene, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, and (4-10 membered heterocycloalkyl)-C₁₋₆ alkylene of R^{b7} are each optionally substituted with 1, 2, 3, 4, 5, 6, 7, or 8 independently selected R^{7A} substituents;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene; R^{7A} is selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, (4-7 membered heterocycloalkyl)-C₁₋₆ alkylene, CN, NO₂, OR^{a71}, SR^{a71}, NHOR^{a71}, C(O)R^{b71}, C(O)NR^{c71}R^{d71} C(O)NR^{c71} (OR^{a71}), C(O)OR^{d71}, OC(O)R^{b71}, OC(O)NR^{c71}R^{d71}, NR^{c71}R^{d71}, NR^{e71}NR^{c71}R^{d71} NR^{c71}C(O)R^{b71}, NR^{c71}C(O)OR^{d71}, NR^{c71}C(O)NR^{c71}R^{d71}, C(═NR^{e71})R^{b71}, C(═NR^{e71})NR^{c71}R^{d71} NR^{c71}C(═NR^{e71})NR^{c71}R^{d71}, NR^{c71}C(═NR^{e71})R^{b71}, NR^{c71}S(O)R^{b71}, NR^{c71}S(O)NR^{c71}R^{d71} NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71}, S(O)₂R^{b71}, S(O)₂NR^{c71}R^{d71}, OS(O)(═NR^{e71})R^{b71}, and OS(O)₂R^{b71}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene of R^{7A} are each optionally substituted with 1, 2, 3, or 4 independently selected R^{M} substituents;
each R^{7A} is selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, (4-7 membered heterocycloalkyl)-C₁₋₆ alkylene, CN, NO₂, OR^{a71}, SR^{a71}, NHOR^{a71}, C(O)R^{b71}, C(O)NR^{c71}R^{d71} C(O)NR^{c71} (OR^{a71}), C(O)OR^{d71}, OC(O)R^{b71}, OC(O)NR^{c71}R^{d71}, NR^{c71}R^{d71}, NR^{c71}NR^{c71}R^{d71} NR^{c71}C(O)R^{b71}, NR^{c71}C(O)OR^{d71}, NR^{c71}C(O)NR^{c71}R^{d71}, C(═NR^{e71})R^{b71}, C(═NR^{e71})NR^{c71}R^{d71} NR^{c71}C(═NR^{e71})NR^{c71}R^{d71}, NR^{c71}C(═NR^{e71})R^{b71}, NR^{c71}S(O)R^{b71}, NR^{c71}S(O)NR^{c71}R^{d71}, NR^{c71}S(O)₂R^{b71}, NR^{c71}S(O)(═NR^{e71})R^{b71}, NR^{c71}S(O)₂NR^{c71}R^{d71}, S(O)R^{b71}, S(O)NR^{c71}R^{d71} S(O)₂R^{b71}, S(O)₂NR^{c71}R^{d71}, OS(O)(═NR^{e71})R^{b71}, and OS(O)₂R^{b71}, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{7A} are each optionally substituted with 1, 2, 3, or 4 independently selected R^{M} substituents;
each R^{a71}, R^{c71}, and R^{d71} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{a71}, R^{c71}, and R^{d71} are each optionally substituted with 1, 2, 3, or 4 independently selected R^{M} substituents;
or any R^{c71} and R^{d71} attached to the same N atom, together with the N atom to which they are attached, form a 5-6 membered heteroaryl or a 4-7 membered heterocycloalkyl group, wherein the 5-6 membered heteroaryl or 4-7 membered heterocycloalkyl group is optionally substituted with 1, 2, 3, or 4 independently selected R^{M} substituents;
each R^{b71} is independently selected from H, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene, wherein the C₁₋₆ alkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkyl-of R^{b71} are each optionally substituted with 1, 2, 3, or 4 independently selected R^{M} substituents;
each R^{e71} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene; and
each R^{M} is independently selected from H, OH, halo, oxo, CN, C(O)OH, NH₂, NO₂, SF₅, C₁₋₆ alkyl, C₁₋₆ alkoxy, C₁₋₆ haloalkoxy, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene.

2. The compound of claim 1, or a pharmaceutically acceptable salt or stereoisomer thereof, having a structure of Formula (II) 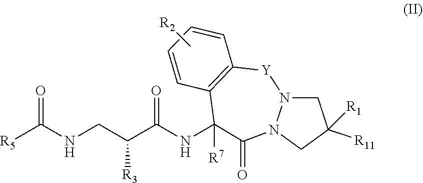
wherein:
Y is CH₂ or C═O;
R₁ is H, C₁-C₆alkyl or halo;
R₂ is H, halo, C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by one or more D or C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo;
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy; and
xxi) —C(═O)NH₂;
each R₆ is independently selected from C₁-C₆alkyl, C₁-C₆alkoxy, C₁-C₆haloalkyl, cyano and halo;
R₁₁ is H, C₁-C₆alkyl or halo; or
R₁ and R₁₁ together with the carbon atom to which they are attached, may form a 3 to 6 membered carbocyclic ring;
R⁷ is selected from D, CD₃, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, (4-10 membered heterocycloalkyl)-C₁₋₆ alkylene, CN, C(O)R^{b7}, C(O)NR^{c7}R^{d7}, C(O)NR^{c7}(ORd^{d7}), C(O)OR^{d7}, C(═NR^{e7})R^{b7}, C(═NR^{e7})NR^{c7}R^{d7}, S(O)R^{b7}, S(O)NR^{c7}R^{d7}, S(O)₂R^{b7}, and S(O)₂NR^{c7}R^{d7} wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R⁷ are each optionally substituted with 1, 2, 3, or 4 independently selected R^{7A} substituents;
each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene;
each R^{b7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene;
R^{7A} is selected from halo, C₁₋₆ alkyl, C₁₋₆ haloalkyl, C₂₋₆ alkenyl, C₂₋₆ alkynyl, phenyl, C₃₋₇ cycloalkyl, 5-6 membered heteroaryl, 4-7 membered heterocycloalkyl, phenyl-C₁₋₆ alkylene, C₃₋₇ cycloalkyl-C₁₋₆ alkylene, (5-6 membered heteroaryl)-C₁₋₆ alkylene, and (4-7 membered heterocycloalkyl)-C₁₋₆ alkylene; and
each R^{7A} is selected from halo, CN, NO₂, OH, C₁₋₆ alkyl, C₁₋₆ haloalkyl, C₁₋₆ alkoxy, and C₁₋₆ haloalkoxy.

3. The compound of claim 1, or a pharmaceutically acceptable salt thereof, having a structure of Formula (IIA), Formula (IIB), Formula (IIC) or Formula (IID): 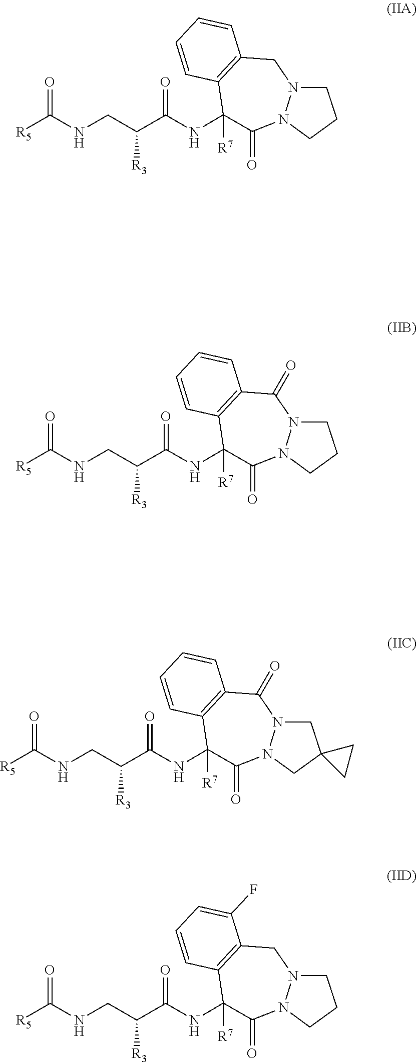
wherein
R₃ is H, C₁-C₆haloalkyl, C₃-C₆cycloalkyl, C₁-C₆alkyl-phenyl or C₁-C₆alkyl optionally substituted with C₁-C₆alkoxy;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by one or more D or C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC1-C₅alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo;
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy; and
xxi) —C(═O)NH₂;
R⁷ is selected from D, CD₃, halo, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, (4-10 membered heterocycloalkyl)-C₁₋₆ alkylene, CN, C(O)R^{b7}, C(O)NR^{c7}R^{d7}, C(O)NR^{c7}(ORd^{d7}), C(O)OR^{d7}, C(═NR^{e7})R^{b7}, C(═NR^{e7})NR^{c7}R^{d7}, S(O)R^{b7}, S(O)NR^{c7}R^{d7}, S(O)₂R^{b7}, and S(O)₂NR^{e7}R^{d7}, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene of R⁷ are each optionally substituted with 1, 2, 3, or 4 independently selected R^{7A} substituents each R^{a7}, R^{c7}, and R^{d7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene;
each R^{b7} is independently selected from H, C₁₋₆ alkyl, C₁₋₆ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene;
each R^{e7} is independently selected from H, OH, CN, C₁₋₆ alkyl, C₁₋₆ alkoxy, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₆ alkylene, C₃₋₁₀ cycloalkyl-C₁₋₆ alkylene, (5-10 membered heteroaryl)-C₁₋₆ alkylene, and (4-10 membered heterocycloalkyl)-C₁₋₆ alkylene; and
each R^{7A} is selected from halo, CN, NO₂, OH, C₁₋₆ alkyl, C₁₋₆ haloalkyl, C₁₋₆ alkoxy, and C₁₋₆ haloalkoxy.

4. A compound of Formula (Ia) or a pharmaceutically acceptable salt or stereoisomer thereof, 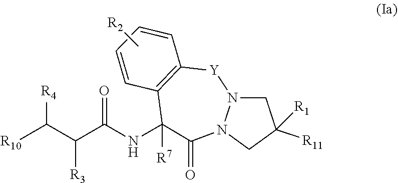
wherein:
Y is CH₂ or C═O;
R₁ is H, C₁-C₆alkyl or halo;
R₂ is C₁-C₆alkyl, C₁-C₆haloalkyl, C₁-C₆alkyloxy, and C₁-C₆haloalkyloxy;
R₃ is H, C₁-C₆haloalkyl, C₃-C₆cycloalkyl, C₁-C₆alkyl-phenyl or C₁-C₆alkyl optionally substituted with C₁-C₆alkoxy;
R₄ is H, C₁-C₆alkyl or C₁-C₆alkyl-phenyl;
R₁₀ is —NHC(═O)R₅, —C(═O)NHR₅ or a 9 or 10 membered bicyclic heteroaryl having 1 to 4 heteroatoms as ring members each independently selected from N, O and S, wherein the bicyclic heteroaryl is unsubstituted or the bicyclic heteroaryl is substituted with one or more R₆;
R₅ is a 5-membered heteroaryl having 1, 2 or 3 heteroatoms as ring members each independently selected from N, O and S, wherein the 5-membered heteroaryl is unsubstituted or the 5-membered heteroaryl is substituted with one or more substituents independently selected from:
i) halo;
ii) amino;
iii) C₃-C₆cycloalkyl optionally substituted by one or more halo;
iv) C₃-C₆cycloalkenyl;
v) C₁-C₆alkyl optionally substituted by C₁-C₆alkoxy, C₃-C₆cycloalkyl or phenyl;
vi) C₁-C₆haloalkyl;
vii) —NHC(═O) C₁-C₆alkyl, wherein the C₁-C₆alkyl is optionally substituted by C₁-C₆alkoxy;
viii) —NHC(═O)—C₁-C₆haloalkyl;
ix) —NHC(═O)—C₃-C₆cycloalkyl;
x) —C(═O)NH—C₁-C₆alkyl, wherein the C₁-C₆alkyl is optionally substituted by one or more D or C₁-C₆alkoxy;
xi) —C(═O)NH—C₁-C₆haloalkyl;
xii) —C(═O)NH—C₃-C₆cycloalkyl;
xiii) —NHC(═O) phenyl, wherein the phenyl is optionally substituted with one or more substituents independently selected from halo and C₁-C₆alkyl;
xiv) —C(═O)NHphenyl, wherein the phenyl is optionally substituted with one or more substituents independently selected from halo and C₁-C₆alkyl;
xv) C₁-C₆alkoxy or C₁-C₆haloalkoxy;
xvi) phenyloxy optionally substituted with one or more halo;
xvii) phenyl optionally substituted by one or more substituents independently selected from halo, —CN, C₁-C₆alkyl, C₁-C₆alkoxy, C₁-C₆haloakoxy and C₁-C₆haloalkyl;
xviii) a 4 to 6-member heterocyclyl optionally substituted with oxo, —C(═O)OC1-C₆alkyl or —C(═O)OC1-C₆cycloalkyl;
xix) a 5 or 6 membered heteroaryl having 1 or 2 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by—OH, C₁-C₆alkoxy or a 4 to 6-member heterocyclyl optionally substituted with oxo;
xx) a 9 or 10 membered bicyclic heteroaryl having 1 to 4 heteroatoms as ring members each independently selected from N, O and S, wherein the heteroaryl is unsubstituted or the heteroaryl is substituted by one or more substituents independently selected from halo, C₁-C₆haloalkyl, C₁-C₆haloalkoxy, C₁-C₆alkoxy, 4 to 6-member heterocyclyl, C₃-C₆cycloalkyl, C₃-C₆cycloalkenyl and a C₁-C₆alkyl optionally substituted by C₁-C₆alkoxy; and
xxi) —C(═O)NH₂;
each R₆ is independently selected from C₁-C₆alkyl, C₁-C₆alkoxy, C₁-C₆haloalkyl, cyano and halo;
R₁₁ is H, C₁-C₆alkyl or halo; or
R₁ and R₁₁ together with the carbon atom to which they are attached, may form a 3 to 6 membered carbocyclic ring; and
R⁷ is H.

5. (canceled)

6. The compound of claim 1, or a pharmaceutically acceptable salt or stereoisomer thereof, wherein R₅ is 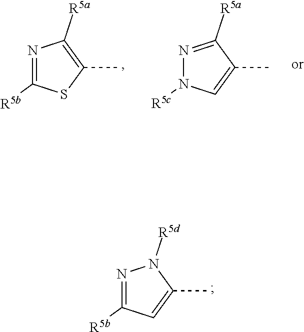
wherein
R^{5a} is C₁-C₆alkyl, C₁-C₆haloalkyl or halo;
R^{5b} is —C(O)—NH—C₁-C₆alkyl, —C(O)NH₂, —C(O)NH-CD₃, —C(O)NH—C₁-C₆haloalkyl, —C(O)NHphenyl, —NHC(═O) C₁-C₆alkyl, C₁-C₆alkoxy, C₁-C₆haloalkoxy, C₃-C₆cycloalkyl, C₃-C₆cycloalkenyl, 4- to 6-membered heterocyclyl, 5- or 6-membered ring heteroaryl; wherein heteroaryl is optionally substituted with halo, C₁-C₆alkyl, C₁-C₆haloakyl, C₁-C₆alkoxy, C₁-C₆haloakoxy or C₃-C₆cycloalkyl; wherein heterocyclyl is optionally substituted with oxo, —C(O)O-C₁-C₆alkyl or —C(O)O—C₅-C₈cycloalkyl; wherein-C(O)NHphenyl is optionally substituted with halo or C₁-C₆alkyl; and each C₁-C₆alkyl is optionally substituted by one or more D;
R^{5c} is 5- or 6-membered ring heteroaryl optionally substituted with halo, C₁-C₆alkyl, C₁-C₆haloakyl, C₁-C₆alkoxy, C₁-C₆haloakoxy or C₃-C₆cycloalkyl; and
R^{5d} is C₁-C₆alkyl or C₁-C₆haloalkyl.

7. The compound according to claim 1, or a pharmaceutically acceptable salt or stereoisomer thereof, wherein R₅ is:

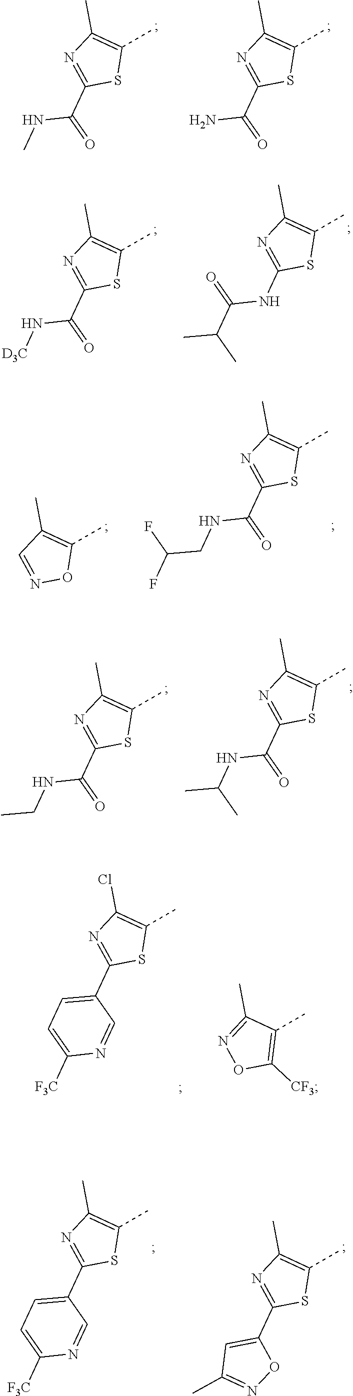

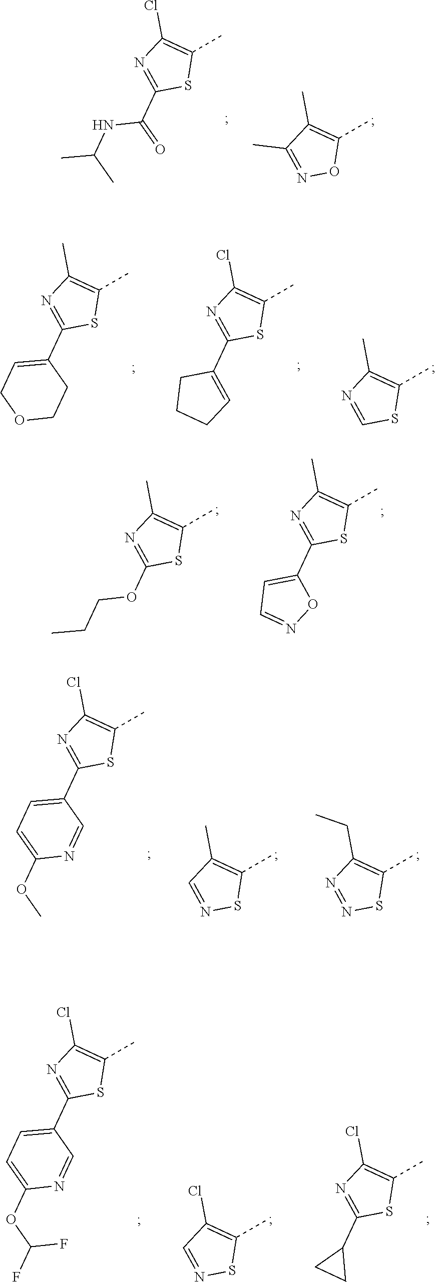

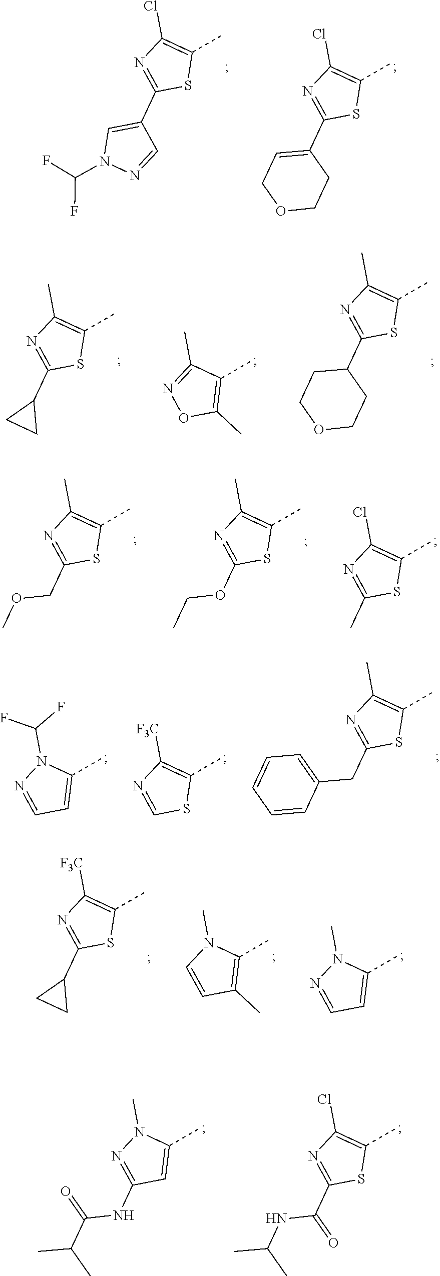

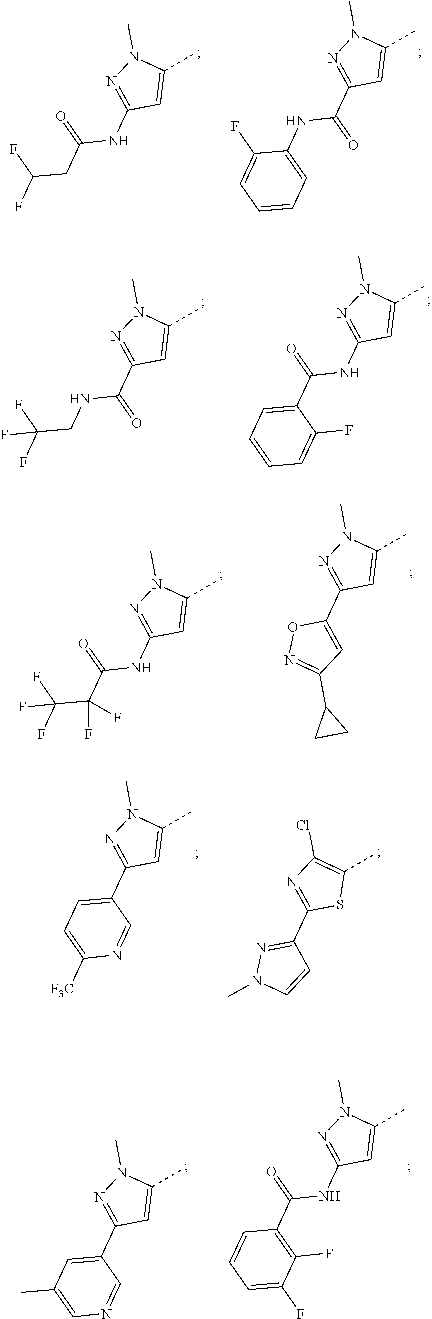

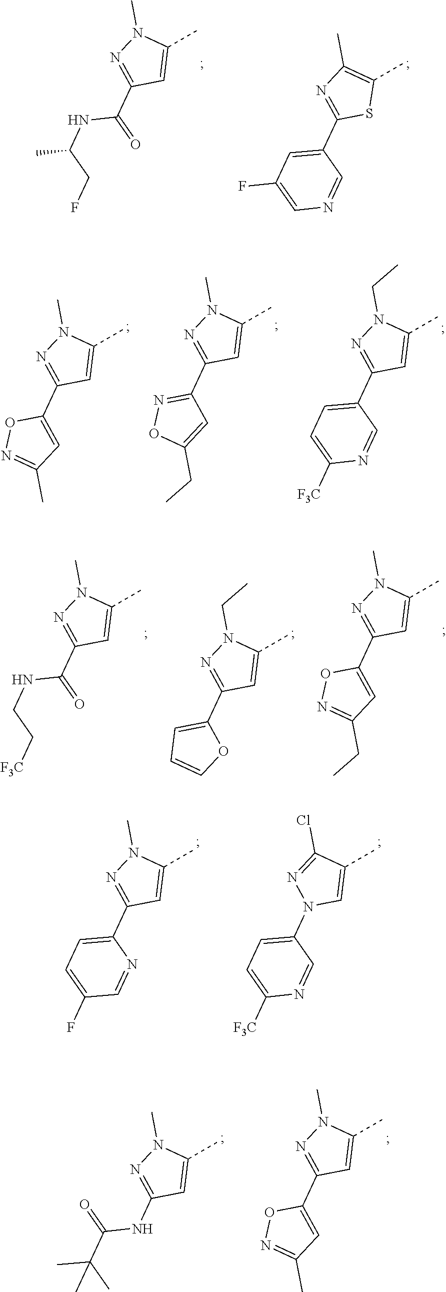

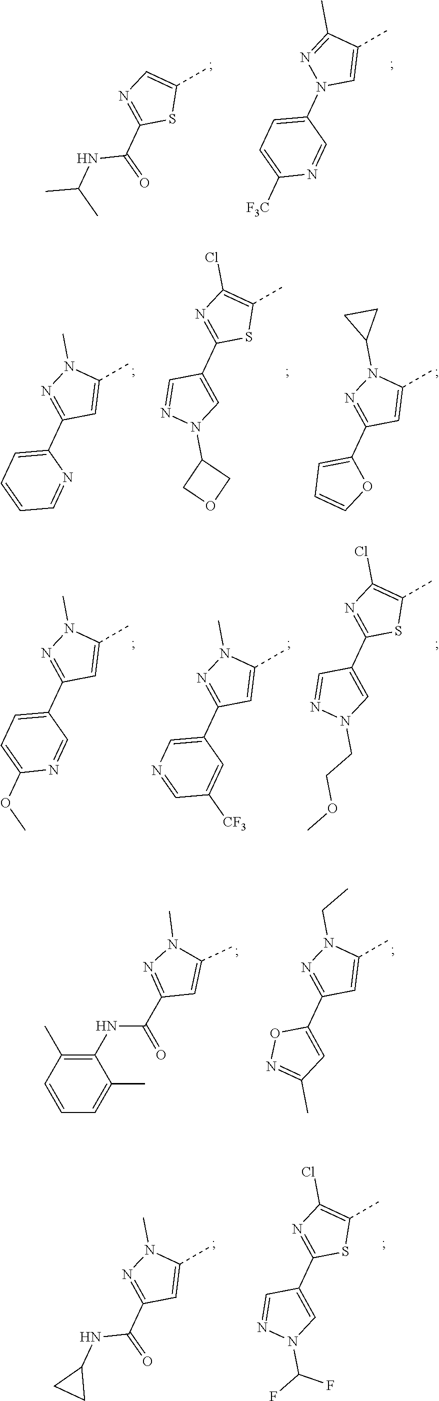

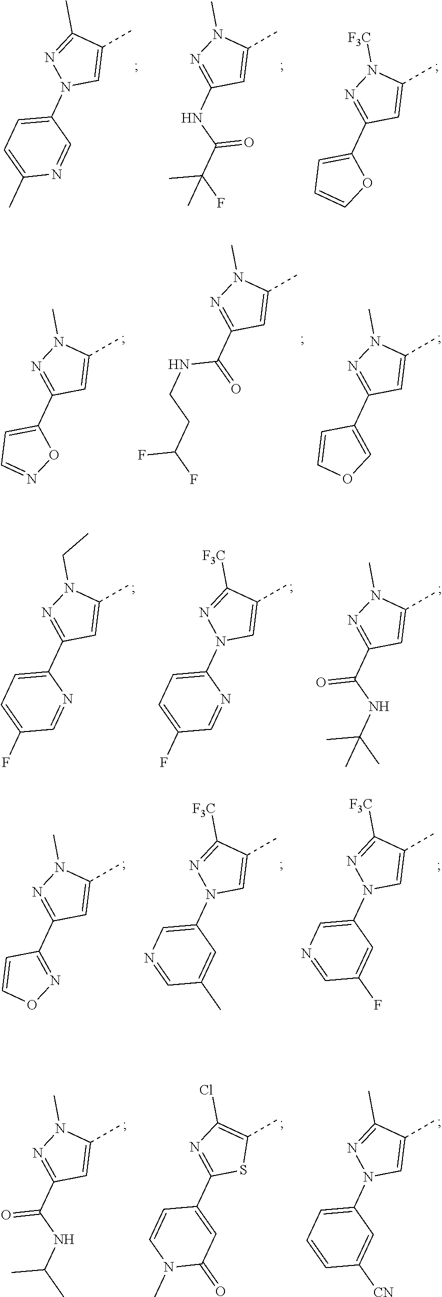

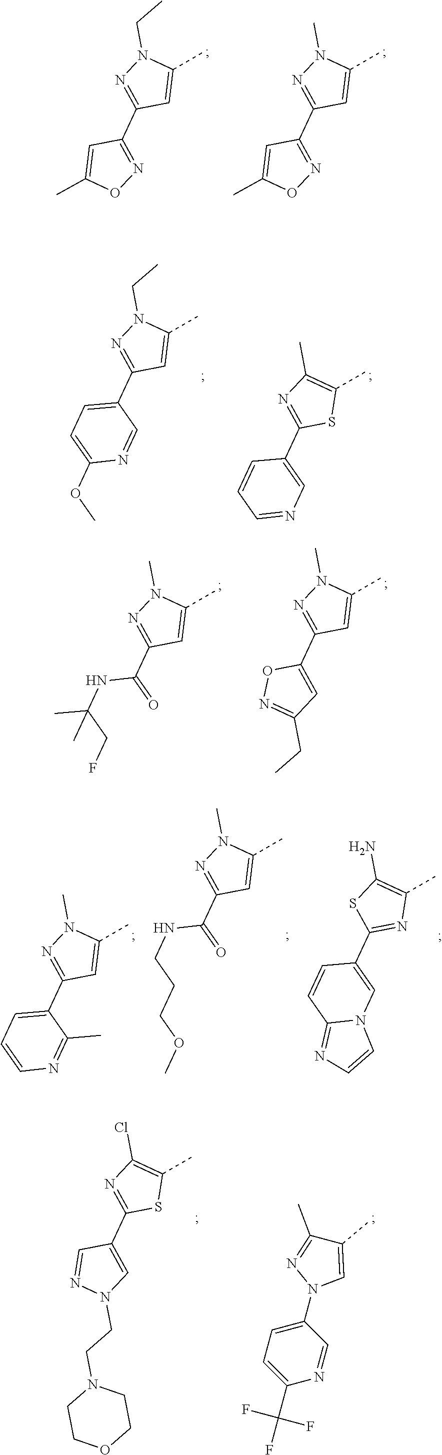

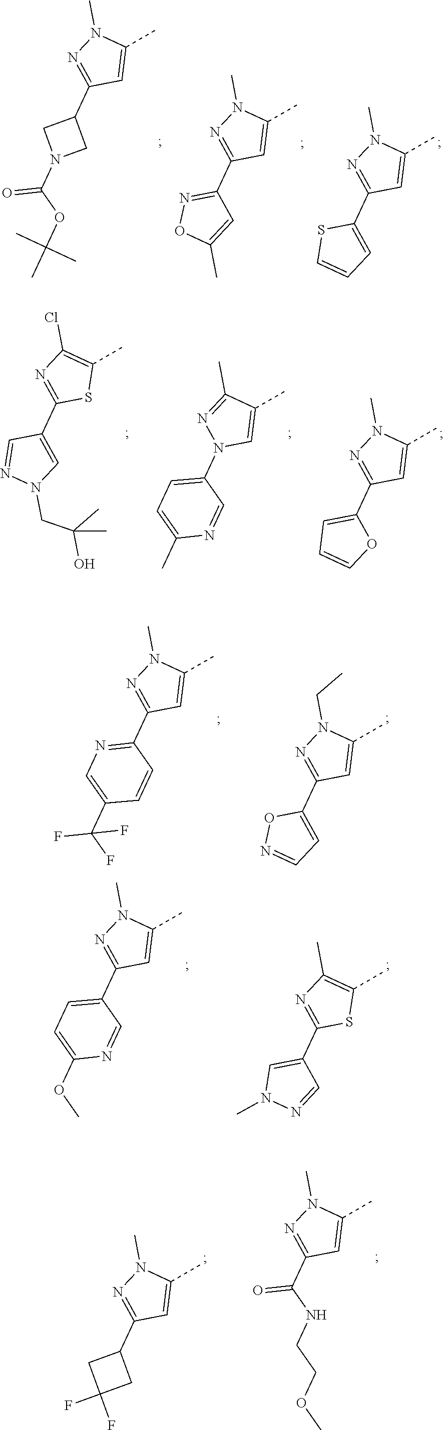

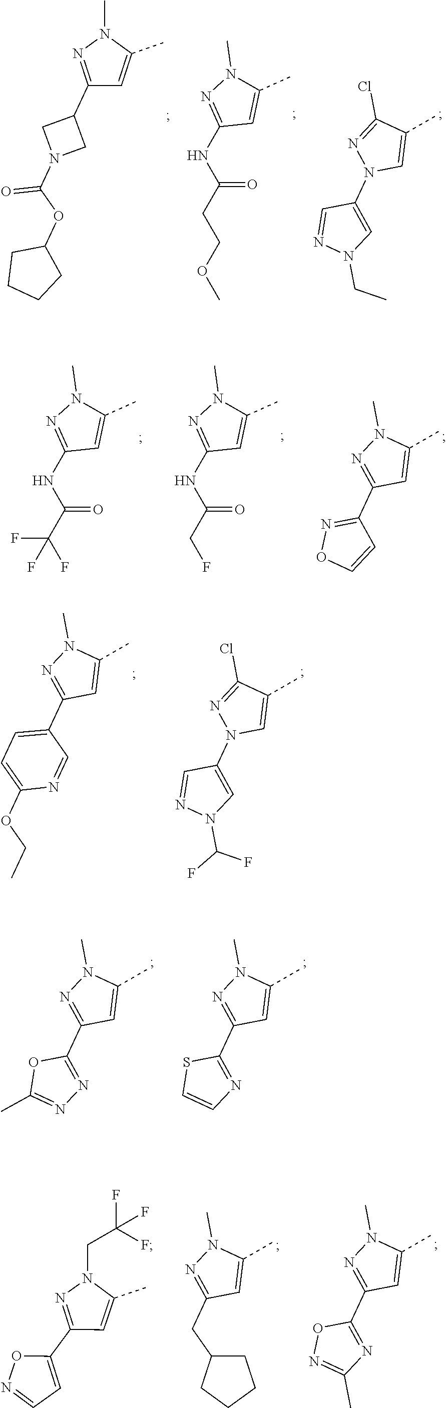

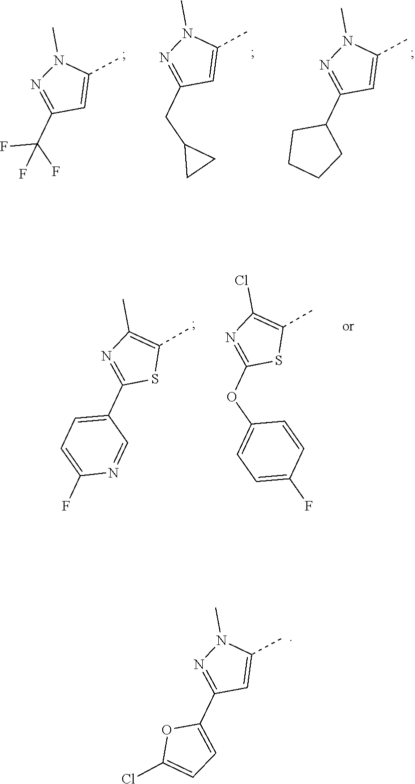

8. The compound of claim 1, wherein R⁷ is D, CD₃, C₁₋₆ alkyl, C₆₋₁₀ aryl-C₁₋₆ alkylene, or (5-10 membered heteroaryl)-C₁₋₆ alkylene.

9-15. (canceled)

16. The compound of claim 1, or a pharmaceutically acceptable salt or stereoisomer thereof, having a structure of Formula (V):

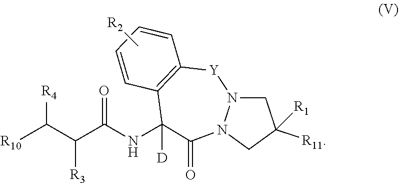

17. The compound of claim 16, or a pharmaceutically acceptable salt or stereoisomer thereof, having a structure of Formula (VA) or Formula (VB):

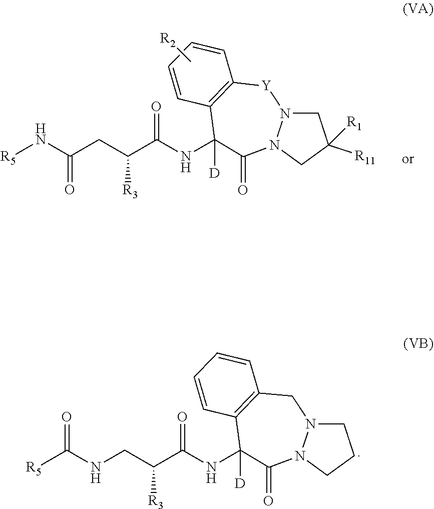

18. (canceled)

19. The compound of claim 17, wherein R₅

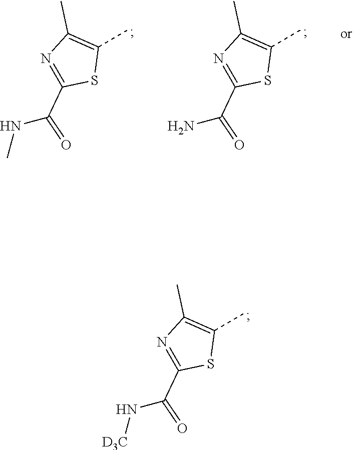

20. The compound of claim 1 selected from:
N²,4-dimethyl-N₆—((R)-2-methyl-3-oxo-3-(((S)-11-oxo-2,3, 10, 11-tetrahydro-1H,5H-benzo[d]pyrazolo[1,2-a][1,2]diazepin-10-yl-10-d)amino)propyl)thiazole-2,5-dicarboxamide;
N²,4-dimethyl-N₆—((R)-2-methyl-3-oxo-3-(((R)-11-oxo-2,3,10, 11-tetrahydro-1H,5H-benzo[d]pyrazolo[1,2-a][1,2]diazepin-10-yl-10-d)amino)propyl)thiazole-2,5-dicarboxamide;
N²,4-dimethyl-N₆—((R)-2-methyl-3-(((S)-10-methyl-11-oxo-2,3, 10, 11-tetrahydro-1H,5H-benzo[d]pyrazolo[1,2-a][1,2]diazepin-10-yl)amino)-3-oxopropyl)thiazole-2,5-dicarboxamide;
N²,4-dimethyl-N₆—((R)-2-methyl-3-(((R)-10-methyl-11-oxo-2,3, 10,11-tetrahydro-1H,5H-benzo[d]pyrazolo[1,2-a][1,2]diazepin-10-yl)amino)-3-oxopropyl)thiazole-2,5-dicarboxamide;
N⁵—((R)-3-(((S)-10-benzyl-11-oxo-2,3, 10, 11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((R)-10-benzyl-11-oxo-2,3, 10, 11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((S)-10-ethyl-11-oxo-2,3,10, 11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((R)-10-ethyl-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N²,4-dimethyl-N₆—((R)-2-methyl-3-oxo-3-(((S)-11-oxo-10-(pyridin-3-ylmethyl)-2,3, 10,11-tetrahydro-1H,5H-benzo[d]pyrazolo[1,2-a][1,2]diazepin-10-yl)amino)propyl)thiazole-2,5-dicarboxamide;
N²,4-dimethyl-N₆—((R)-2-methyl-3-oxo-3-(((R)-11-oxo-10-(pyridin-3-ylmethyl)-2,3, 10,11-tetrahydro-1H,5H-benzo[d]pyrazolo[1,2-a][1,2]diazepin-10-yl)amino)propyl)thiazole-2,5-dicarboxamide;
N⁵—((R)-3-(((S)-10-(4-fluorobenzyl)-11-oxo-2,3, 10, 11-tetrahydro-1H,5H-benzo[d]pyrazolo[1,2-a][1,2]diazepin-10-yl)amino)-2-methyl-3-oxopropyl)-2,4-dimethylthiazole-2,5-dicarboxamide;
N⁵—((R)-3-(((R)-10-(4-fluorobenzyl)-11-oxo-2,3,10, 11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N²,4-dimethyl-N⁵—((R)-2-methyl-3-(((S)-10-(methyl-d₃)-11-oxo-2,3, 10, 11-tetrahydro-1H,5H-benzo[d]pyrazolo[1,2-a][1,2]diazepin-10-yl)amino)-3-oxopropyl)thiazole-2,5-dicarboxamide; and
N²,4-dimethyl-N⁵—((R)-2-methyl-3-(((R)-10-(methyl-d₃)-11-oxo-2,3,10,11-tetrahydro-1H,5H-benzo[d]pyrazolo[1,2-a][1,2]diazepin-10-yl)amino)-3-oxopropyl)thiazole-2,5-dicarboxamide;
N²,4-dimethyl-N₆—((R)-2-methyl-3-oxo-3-(((S)-11-oxo-7-(trifluoromethyl)-2,3, 10, 11-tetrahydro-1H,5H-benzo[d]pyrazolo[1,2-a][1,2]diazepin-10-yl)amino)propyl)thiazole-2,5-dicarboxamide;
N²,4-dimethyl-N₆—((R)-2-methyl-3-oxo-3-(((R)-11-oxo-7-(trifluoromethyl)-2,3, 10, 11-tetrahydro-1H,5H-benzo[d]pyrazolo[1,2-a][1,2]diazepin-10-yl)amino)propyl)thiazole-2,5-dicarboxamide;
N²,4-dimethyl-N₆—((R)-2-methyl-3-oxo-3-(((S)-11-oxo-7-(trifluoromethyl)-2,3, 10,11-tetrahydro-1H,5H-benzo[d]pyrazolo[1,2-a][1,2]diazepin-10-yl-10-d)amino)propyl)thiazole-2,5-dicarboxamide;
N²,4-dimethyl-N₆—((R)-2-methyl-3-oxo-3-(((R)-11-oxo-7-(trifluoromethyl)-2,3, 10, 11-tetrahydro-1H,5H-benzo[d]pyrazolo[1,2-a][1,2]diazepin-10-yl-10-d)amino)propyl)thiazole-2,5-dicarboxamide;
N⁵—((R)-3-(((S)-7-methoxy-11-oxo-2,3, 10, 11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((R)-7-methoxy-11-oxo-2,3,10,11-tetrahydro-1H,5H-benzo[d]pyrazolo[1,2-a][1,2]diazepin-10-yl)amino)-2-methyl-3-oxopropyl)-N²,4-dimethylthiazole-2,5-dicarboxamide;
N⁵—((R)-3-(((S)-7-fluoro-11-oxo-2,3, 10, 11-tetrahydro-1H,5H-benzo[d]pyrazolo[1,2-a][1,2]diazepin-10-yl-10-d)amino)-2-methyl-3-oxopropyl)-N²,4-dimethylthiazole-2,5-dicarboxamide;
N⁵—((R)-3-(((R)-7-fluoro-11-oxo-2,3,10,11-tetrahydro-1H,5H-benzo[d]pyrazolo[1,2-a][1,2]diazepin-10-yl-10-d)amino)-2-methyl-3-oxopropyl)-N²,4-dimethylthiazole-2,5-dicarboxamide;
N⁵—((R)-3-(((S)-7-chloro-11-oxo-2,3, 10, 11-tetrahydro-1H,5H-benzo[d]pyrazolo[1,2-a][1,2]diazepin-10-yl-10-d)amino)-2-methyl-3-oxopropyl)-N²,4-dimethylthiazole-2,5-dicarboxamide; and
N⁵—((R)-3-(((R)-7-chloro-11-oxo-2,3,10, 11-tetrahydro-1H,5H-benzo[d]pyrazolo[1,2-a][1,2]diazepin-10-yl-10-d)amino)-2-methyl-3-oxopropyl)-N₂, 4-dimethylthiazole-2,5-dicarboxamide.
or a pharmaceutically acceptable salt thereof.

21-23. (canceled)

24. A compound, or a pharmaceutically acceptable salt or stereoisomer thereof, wherein the compound is
4-methyl-N₆—((R)-2-methyl-3-oxo-3-(((S)-11-oxo-2,3,10,11-tetrahydro-1H,5H-benzo[d]pyrazolo[1,2-a][1,2]diazepin-10-yl)amino)propyl)thiazole-2,5-dicarboxamide;
4-methyl-N₆—((R)-2-methyl-3-oxo-3-(((S)-11-oxo-2,3, 10, 11-tetrahydro-1H,5H-benzo[d]pyrazolo[1,2-a][1,2]diazepin-10-yl-10-d)amino)propyl)thiazole-2,5-dicarboxamide;
4-methyl-N₆—((R)-2-methyl-3-oxo-3-(((R)-11-oxo-2,3,10, 11-tetrahydro-1H,5H-benzo[d]pyrazolo[1,2-a][1,2]diazepin-10-yl-10-d)amino)propyl)thiazole-2,5-dicarboxamide;
4-methyl-N₂-(methyl-da)-N⁵—((R)-2-methyl-3-oxo-3-(((S)-11-oxo-2,3, 10, 11-tetrahydro-1H,5H-benzo[d]pyrazolo[1,2-a][1,2]diazepin-10-yl)amino)propyl)thiazole-2,5-dicarboxamide;
4-methyl-N₂-(methyl-d₃)—N⁵—((R)-2-methyl-3-oxo-3-(((S)-11-oxo-2,3,10,11-tetrahydro-1H,5H-benzo[d]pyrazolo[1,2-a][1,2]diazepin-10-yl-10-d)amino)propyl)thiazole-2,5-dicarboxamide; or
4-methyl-N₂-(methyl-d₃)—N⁵—((R)-2-methyl-3-oxo-3-(((R)-11-oxo-2,3,10,11-tetrahydro-1H,5H-benzo[d]pyrazolo[1,2-a][1,2]diazepin-10-yl-10-d)amino)propyl)thiazole-2,5-dicarboxamide;
or a pharmaceutically acceptable salt thereof.

25. (canceled)

26. A pharmaceutical composition comprising a therapeutically effective amount of a compound according to claim 1, or a pharmaceutically acceptable salt or stereoisomer thereof, and one or more pharmaceutically acceptable carriers.

27. (canceled)

28. A method of treating a disease or disorder mediated by SPPL2a activity in a subject, wherein the method comprises administering to the subject a therapeutically effective amount of a compound according to claim 1; or a pharmaceutically acceptable salt or stereoisomer thereof.

29-30. (canceled)

31. The method according to claim 28,, wherein the disease or disorder is an autoimmune disease or disorder, a pemphigus disorder, or diabetes.

32-36. (canceled)

37. The method according to claim 28, wherein the disease or disorder is selected from Sjogren's disease, systemic lupus erythematosus (SLE), arthritis, lupus nephritis, systemic sclerosis, multiple sclerosis (MS), autoimmune hepatitis, uveitis, myasthenia gravis, Hashimoto thyroiditis, thrombocytopenia purpura, myocarditis, atopic dermatitis, Goodpasture syndrome, hidradenitis suppurativa, ANCA vasculitis, dermatomyositis, primary biliary cirrhosis, polymyositis, temporal arteritis, alopecia areata, autoimmune hemolytic anemia, insulin resistance, a metabolic disease, fatty liver disease, hyperlipidemia, atherosclerosis, hypertension, stroke, gall bladder disease, and obesity.

38. The method according to claim 37, wherein the disease or disorder is arthritis or systemic lupus erythematosus (SLE).

39-42. (canceled)

43. The method according to claim 28, wherein the disease or disorder is acute and chronic graft versus host disease (GvHD).

44-45. (canceled)

46. A method of treating a condition selected from hidradenitis suppurativa, arthritis, cutaneous lupus, a pemphigus disorder, ANCA vasculitis, diabetes, dermatomyositis, primary biliary cirrhosis, polymyositis, temporal arteritis, alopecia areata, autoimmune hemolytic anemia, insulin resistance, a metabolic disease, fatty liver disease, hyperlipidemia, atherosclerosis, hypertension, stroke, gall bladder disease, and obesity in a subject in need thereof, comprising administering to the subject a therapeutically effective amount of N²,4-dimethyl-N⁵—((R)-2-methyl-3-oxo-3-(((S)-11-oxo-2,3, 10, 11-tetrahydro-1H,5H-benzo[d]pyrazolo[1,2-a][1,2]diazepin-10-yl)amino)propyl)thiazole-2,5-dicarboxamide, or a pharmaceutically acceptable salt thereof.

47. The method of claim 46, wherein arthritis is juvenile arthritis or psoriatic arthritis; the pemphigus disorder is bullous pemphigoid; the diabetes is or type 2 diabetes; and the polymyositis is juvenile polymyositis.

48. The method of claim 46, wherein the condition is cutaneous lupus.

49-53. (canceled)
